(19) United States
(12) Patent Application Publication
(10) Pub. No.: US 2021/0268126 A1
(43) Pub. Date: Sep. 2, 2021

(54) TREATING SPINAL CORD INJURY (SCI) AND BRAIN INJURY USING GSX1

(71) Applicant: Rutgers, The State University of New Jersey, New Brunswick, NJ (US)

(72) Inventors: Li Cai, Warren, NJ (US); Misaal Patel, Avenel, NJ (US); Yi Lisa Lyu, Warren, NJ (US)

(73) Assignee: Rutgers, The State University of New Jersey, New Brunswick, NJ (US)

(21) Appl. No.: 17/268,664

(22) PCT Filed: Aug. 16, 2019
(86) PCT No.: PCT/US2019/046881

§ 371 Date: Feb. 16, 2021

Related U.S. Application Data
(60) Provisional application No. 62/721,679, filed on Aug. 23, 2018.

(51) Int. Cl. A61K 48/00; A61K 38/17; A61K 47/64; A61K 9/00; A61P 25/28; C12N 5/0793

(52) U.S. Cl. CPC A61K 48/0058; A61K 38/1709; A61K 47/6455; C12N 2510/00; A61P 25/28; C12N 5/0619; A61K 9/0019

(57) ABSTRACT

Methods for treating a neurological disorder, such as a traumatic spinal cord injury or traumatic brain injury, or a disorder such as Parkinson's disease or multiple sclerosis are provided. Such methods include administering a therapeutically effective amount of Gsx1 protein (such as a Gsx1-cell penetrating peptide fusion protein), or a nucleic acid molecule encoding such a protein (for example as part of a viral vector), thereby treating the neurological disorder.

### CROSS-REFERNCE TO RELATED APPLICATION

This application claims priority to U.S. Provisional Application No. 62/721,679 filed Aug. 23, 2018, herein incorporated by reference in its entirety.

### ACKNOWLEDGMENT OF GOVERNMENT SUPPORT

This invention was made with government support under P200A150131 awarded by The US Department of Education, and under T32GM008339 awarded by The National Institutes of Health and under P200A150131 awarded by the U.S. Department of Education. The government has certain rights in the invention.

### FIELD

The disclosure provides methods for treating a neurological disorder, such as a traumatic spinal cord injury or brain injury, or a disorder such as Parkinson's disease, by administering a therapeutically effective amount of Gsx1 protein (such as a Gsx1-cell penetrating peptide fusion protein) or nucleic acid molecule encoding Gsx1, thereby treating the neurological disorder.

### BACKGROUND

According to the National Spinal Cord Injury Statistical Center, there are approximately 17,000 new incidents of spinal cord injury (SCI) in the United States each year. Major causes of SCI include vehicle crashes, job-related injuries, falls, acts of violence, and sports. SCI causes a significant loss of oligodendrocytes, neurons, and astrocytes, leading to morbidity and disability that require long term care. Despite extensive research, neuroregeneration and functional recovery after SCI have been extremely difficult.

To restore function after SCI, repair and reconstruction of the damaged local circuitry is needed. Major hurdles in neural regeneration include a limited level of neurogenesis in the adult spinal cord, an inflammatory microenvironment that inhibits neurogenesis, axon regeneration, neuronal relay formation, and myelination at the injury site (Tran et al., Physiol Rev 98:881-917 (2018)). SCI activates endogenous neural stem and progenitor cells (NSPCs) that reside around the central canal in the ependymal region (Lacroix et al., PLoS One 9:e85916 (2014)) , which provides potential cell source for damage repair and regeneration. However, adult NSPCs in the spinal cord largely differentiate into astrocytes and oligodendrocytes, with only a very small portion into neurons (Sabelstrom et al., Exp Neurol 260:44-49 (2014)). Efforts have been made to repair and regenerate the damaged spinal cord by stem cell therapy and forced expression of neurogenic transcription factors (Sox2, NeuroD1, and Olig2) to generate neurons in injured spinal cord (Chen et al., Brain Res Bull 135, 143-148 (2017)). However, these approaches provide limited or no functional improvement. Furthermore, injury-induced reactive astrocytes produce chondroitin sulfate proteoglycans (CSPGs), which prevent axonal growth and sprouting, and result in permanent functional deficits. Attempts to attenuate glial scar formation have shown the potential of this approach to promote axonal regeneration. Nevertheless, reducing scar tissue alone does not promote sufficient functional improvement (Dias et al., Cell 173:153-165 e122 (2018)). In addition, SCI induces over-inhibition by the GABAergic neurons causing the spared axons non-functional (Courtine et al., Nat Med 14:69-74 (2008)). By reducing the excitability of inhibitory interneurons or re-establishing the excitation/inhibition ratio, the dormant relay pathways can be reactivated, which leads to an improved locomotor function (Chen et al., Cell 174:1599 (2018)).

Genomic Screened Homeo Box 1 (Gsx1 or Gsh1) is a neurogenic factor highly expressed in the central nervous system at the embryonic stage (Gong et al., Nature 425:917-925 (2003)). During the development of the spinal cord, Gsx1 and its homolog Gsx2 regulate proliferation and differentiation of neural stem/progenitor cells (NSPCs). In the adult spinal cord, Gsx1 expression is low or not detected (Chen et al., PLoS One 8, e72567 (2013)).

### SUMMARY

It is shown herein that use of a lentivirus-mediated gene expression system to transduce Gsx1 into the adult mouse spinal cord with a lateral hemisection injury, results in Gsx1 expression that promotes cell proliferation and activation of NSPCs at the injury site. Furthermore, lentivirus-mediated Gsx1 expression at or near the injury site (Gsx1 treatment) increases the number of glutamatergic and cholinergic neurons and decreases the number of GABAergic interneurons. Gsx1 treatment attenuated glial scar formation and dramatically improved locomotor function in the injured mice. Genome-wide transcriptome analysis reveals that Gsx1 treatment induces the Notch signaling pathway, which correlates with NSPC activation, neuronal differentiation, and provides molecular insight for Gsx1-mediated functional recovery.

Based on these observations, provided herein are methods of treating a neurological disorder in a mammalian subject, such as a human or veterinary subject. The neurological disorder can be a spinal cord injury, a brain injury, or both, such as one resulting from head and/or spinal cord trauma, such as one caused by a vehicle crash, fall, act of violence, or sports. In some examples, the neurological disorder is a neurodegenerative disease, such as Parkinson's disease, Alzheimer's disease, stroke, ischemia, epilepsy, Huntington's disease, multiple sclerosis, or amyotrophic lateral sclerosis. Such methods can increase neurogenesis, decrease inflammation, decrease cell death, reduce astrogliosis, reduce glial scaring, increase locomotion of the subject, or combinations thereof. In some examples, the method increases neurogenesis, reduces astrogliosis, reduces glial scaring, and increases locomotion of the subject.

In some examples, the methods include administering to the subject a therapeutically effective amount of a Gsx1 protein (such as one that includes at least 80%, at least 85%, at least 90%, at least 95%, at least 98%, at least 99%, or 100% sequence identity to SEQ ID NO: 2 or 4) or administering to the subject a therapeutically effective amount of a nucleic acid molecule encoding Gsx1 (such as one that includes at least 80%, at least 85%, at least 90%, at least 95%, at least 98%, at least 99%, or 100% sequence identity to SEQ ID NO: 1 or 3), thereby treating the neurological disorder. In one example, the Gsx1 protein administered is a Gsx1 fusion protein, such as one that includes a Gsx1 domain (such as one that includes at least 80%, at least 85%, at least 90%, at least 95%, at least 98%, at least 99%, or 100% sequence identity to SEQ ID NO: 2 or 4) and a cell penetrating peptide (CPP) domain (such as one that includes at least 80%, at least 85%, at least 90%, at least 95%, at least 98%, at least 99%, or 100% sequence identity to any of SEQ ID NOS: 61-79), wherein the Gsx1 domain and the CPP domain can be linked indirectly (e.g., via a linker, such as SEQ ID NO: 80 or 81) or directly. In one example, the nucleic acid molecule encoding Gsx1 encodes a Gsx1-CPP fusion protein, such as one that encodes a Gsx1 domain (such as one that includes at least 80%, at least 85%, at least 90%, at least 95%, at least 98%, at least 99%, or 100% sequence identity to SEQ ID NO: 1 or 3, or encodes a protein comprising at least 80%, at least 85%, at least 90%, at least 95%, at least 98%, at least 99%, or 100% sequence identity to SEQ ID NO: 2 or 4) operably linked to a CPP domain (such as one that encodes a CPP domain having at least 80%, at least 85%, at least 90%, at least 95%, at least 98%, at least 99%, or 100% sequence identity to any of SEQ ID NOS: 61-79). In some examples, the Gsx1 protein, Gsx1-CPP fusion protein, or the nucleic acid molecule encoding one of these proteins, is isolated or purified. Administration can include injection, such as injection into the CNS (e.g., spinal cord or brain).

In some examples, the nucleic acid molecule encoding Gsx1 (such as a Gsx1-CPP fusion protein) includes or is part of a plasmid or viral vector, such as a lentiviral vector or adeno-associated viral (AAV) vector. The nucleic acid molecule encoding Gsx1 (such as a Gsx1-CPP fusion protein) can be operably linked to a promoter, and enhancer, or both. Exemplary promoters include a constitutive promoter (e.g., CMV) or a central nervous system (CNS)-specific promoter (e.g., a synapasin 1 (Syn1) promoter, glial fibrillary acidic protein (GFAP) promoter, nestin (NES) promoter, myelin-associated oligodendrocyte basic protein (MOBP) promoter, myelin basic protein (MBP) promoter, tyrosine hydroxylase (TH) promoter, or a forkhead box A2 (FOXA2) promoter). An exemplary enhancer is a neural-specific enhancer, such as Notch1CR2 (e.g., Tzatzalos et al., Dev Biol. 372(2):217-28, 2012) or Olig2CR5 (Hao et al., Dev Biol. 393(1):183-93, 2014).

One or more doses of the Gsx1 protein, Gsx1-CPP fusion protein, or nucleic acid molecule encoding Gsx1 (such as a Gsx1-CPP fusion protein) can be administered. In one example, at least two separate administrations of the therapeutically effective amount of Gsx1 protein, Gsx1-CPP fusion protein, or nucleic acid molecule encoding Gsx1 (such as a Gsx1-CPP fusion protein) are given to the subject, such as separated by at least 1 week, at least 2 weeks, at least 3 weeks, at least 4 weeks, at least 1 month, at least 2 months, at least 3 months, at least 6 months, at least 9 months, or at least one year. In some examples, the Gsx1 protein, Gsx1-CPP fusion protein, or nucleic acid molecule encoding Gsx1 (such as a Gsx1-CPP fusion protein) is administered within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the neurological disorder (e.g., within this amount of time following the traumatic event leading to the neurological disorder). Other neurological disorder therapeutic agents can also be administered.

Also provided are compositions, which can be used with the disclosed methods. In one example, the composition includes an isolated Gsx1 protein comprising at least 80%, at least 85%, at least 90%, at least 95%, at least 98%, at least 99%, or 100% sequence identity to SEQ ID NO: 2 or 4 and a liposome, wherein the Gsx1 protein is encapsulated in the liposome. In one example, the composition includes a fusion protein that includes (1) a Gsx1 domain comprising at least 80%, at least 85%, at least 90%, at least 95%, at least 98%, at least 99%, or 100% sequence identity to SEQ ID NO: 2 or 4 and (2) a cell penetrating peptide domain (such as one having at least 80%, at least 85%, at least 90%, at least 95%, at least 98%, at least 99%, or 100% sequence identity to any of SEQ ID NOS: 61-79). Such a fusion protein can be encapsulated in a liposome. In one example, the composition includes a nucleic acid molecule encoding a Gsx1 comprising at least 80%, at least 85%, at least 90%, at least 95%, at least 98%, at least 99%, or 100% sequence identity to SEQ ID NO: 1 or 3 and a liposome, wherein such nucleic acid molecule is encapsulated in the liposome and includes or is part of a plasmid or viral vector, such as a lentiviral vector or adeno-associated viral (AAV) vector. In one example, the composition includes a nucleic acid molecule encoding a Gsx1-CPP fusion protein, such as such as one that encodes a Gsx1 domain (such as one that includes at least 80%, at least 85%, at least 90%, at least 95%, at least 98%, at least 99%, or 100% sequence identity to SEQ ID NO: 1 or 3, or encodes a protein comprising at least 80%, at least 85%, at least 90%, at least 95%, at least 98%, at least 99%, or 100% sequence identity to SEQ ID NO: 2 or 4) operably linked to a CPP domain (such as one that encodes a CPP domain having at least 80%, at least 85%, at least 90%, at least 95%, at least 98%, at least 99%, or 100% sequence identity to any of SEQ ID NOS: 61-79), and a liposome, wherein such nucleic acid molecule is encapsulated in the liposome and includes or is part of a plasmid or viral vector, such as a lentiviral vector or adeno-associated viral (AAV) vector. The disclosed compositions can further include other materials, such as a pharmaceutically acceptable carrier, such as water or saline. The disclosed compositions can further include other materials, such as a pharmaceutically acceptable carrier, such as water or saline.

The foregoing and other objects and features of the disclosure will become more apparent from the following detailed description, which proceeds with reference to the accompanying figures.

### BRIEF DESCRIPTION OF THE DRAWINGS

FIGS. 1A-1G: Gsx1 expression promotes cell proliferation in the injured spinal cord. Lateral hemisection SCI was performed on 8-12 weeks old mice around T9-T10 level immediately followed by the injection of lentivirus encoding Gsx1 along with RFP reporter (lenti-Gsx1-RFP). Lentivirus encoding only the reporter RFP was used as a control (lenti-Ctrl-RFP). Spinal cord tissues were analyzed by immunohistochemistry, RNA-Seq, Ingenuity Pathway Analysis (IPA), and RT-qPCR analysis; scale bar=100 μm (A). Confocal images of sagittal sections of spinal cord tissue at 3 DPI show the expression of viral reporter RFP and cell proliferation marker Ki67 (n=3 for Sham and n=6 for SCI+Ctrl and SCI+Gsx1). Arrows indicate Ki67+/RFP+ co-labeled cells. Images in the bottom left corner show a higher magnification z-stack view of the area denoted by a dashed white box. Scale bar=20 μm (B). Quantification of all Ki67+ cells (C) and Ki67+/RFP+ cells (D). RT-qPCR analysis shows Ki67 mRNA expression at 3 DPI, normalized to the Sham; n=4 (E). List of differentially expressed genes that are known to inhibit proliferation between after lenti-Gsx1 treatment compared to lenti-Ctrl treatment from RNA-Seq analysis (n=3) (F). Gene expression box plot, generated by STAR and edgeR, know to promote cell proliferation; *=differentially significant (G). Each dot represents the gene expression as log 2(count per million) for one biological replicate sample. Mean±SEM; *=p<0.05; Students' t-test.

FIGS. 2A-2E: Transduction of lenti-Gsx1-RFP is successful in delivering and overexpressing Gsx1 after SCI. Hemisection SCI was performed on 8-12 weeks old mice around T10. Immediately after lentivirus injection encoding Ctrl or Gsx1 gene along with RFP reporter. Animals were harvested 3 DPI (A) and 7 DPI (B) and sagittal sections are immunostained with Gsx1 antibody. Arrows in sagittal sections show co-expression of RFP and Gsx1 (green). Montage on the right of each of the image indicates small region (white box) of sagittal sections with separate channels (DAPI, RFP, and Gsx1) to indicate co-expression. Scale bar=50 Quantification of virally transduced cells co-labeled with Gsx1 at 3 DPI (C) and 7 DPI (D). (E) RT-qPCR analysis indicating Gsx1 mRNA expression at 3 DPI, normalized to the Sham. n=3; Mean±SEM; *=p<0.05; One-way ANOVA and Tukey post-hoc analysis. DPI =days post injury.

FIGS. 3A-3C: RNA-Seq Analysis. (A) Number of biological replicates used for each group (SCI+Ctrl and SCI+Gsx1) at 3 different times points (3 DPI, 14 DPI, and 35 DPI) for RNA-Seq analysis. (B) Total number of differentially expressed genes (DEGs; p<0.05) that are upregulated and downregulated at 3 DPI, 14 DPI, and 35 DPI. (C) Volcano plot at 3 DPI, 14 DPI, and 35 DPI indicating differentially expressed genes.

FIGS. 3D-3F. Top 40 differentially expressed genes (DEGs) at (D) 3 DPI, (E) 14 DPI, and (F) 35 CPI. Heatmap of the top 40 DEGs between SCI+Ctrl and SCI+Gsx1 group at 3 DPI, 14 DPI and 35 DPI, respectively. Blue indicates downregulation and yellow indicates upregulation of the gene expression; n=3.

FIG. 4: Functional enrichment of gene ontology (GO) terms for differentially expressed genes (DEGs) at 3 DPI. Enrichment terms for biological process represented as a scatter plot in a two dimensional semantic space using REVIGO. Circle size indicates the log 10(p-value) of the GO terms.

FIGS. 5A-5I: Gsx1 expression increases NSPC activation after SCI. (A) Confocal images of sagittal sections of spinal cord tissues at 3 DPI show the expression of viral reporter RFP and NSPC marker Nestin (n=3 for Sham and n=6 for SCI+Ctrl and SCI+Gsx1). Arrows indicate Nestin+/RFP+ co-labeled cells. Images in the bottom left corner show a higher magnification z-stack view of the area denoted by a dashed white box. Scale bar=20 (B) Quantification of all Nestin+ cells and (C) Nestin+/RFP+ co-labeled cells. (D) RT-qPCR analysis shows Nestin mRNA expression at 3 DPI, normalized to the Sham; n=4. (E) List of differentially expressed genes that promote Notch signaling after lenti-Gsx 1 treatment compared to lenti-Ctrl treatment from RNA-Seq analysis. (F) Confocal images of sagittal sections of spinal cord tissues at 3 DPI show the expression of viral reporter RFP and NSPC marker Notch1 (n=4 for SCI+Ctrl and SCI+Gsx1). Arrows indicate Notch1+/RFP+ co-labeled cells. Images in the bottom left corner show a higher magnification z-stack view of the area denoted by a dashed white box. Scale bar=20 (G) Quantification of all Notch1+ cells among RFP+ cells. (H) RT-qPCR analysis of the genes involved in the Notch signaling pathway (Notch1, Nrarp, Jag1, Del1, and Hes1). (I) Gene expression box plot of the genes associated with stem cells and Nanog signaling pathway (e.g., Akt2, Map2k2, Pik3cd, Pik3cg, and Rap2b). Each dot represents the gene expression as log 2(count per million) for one biological replicate sample. Mean±SEM; *=p<0.05; Students' t-test and One-way ANOVA followed by Tukey post-hoc test.

FIGS. 6A-6G: Gsx1 induces neurogenesis in the adult spinal cord after SCI. Confocal images of sagittal sections of spinal cord tissues at 14 DPI show the expression of viral reporter RFP and early neuronal marker (A) Doublecortin DCX, (B) astrocyte marker GFAP, and (C) oligodendrocyte progenitor marker PDGFRa. Arrows indicate cell marker+/RFP+ co-labeled cells. Images in the bottom left corner show a higher magnification z-stack view of the area denoted by a dashed white box. Scale bar=20 μm. (D) Quantification of virally transduced cells co-labeled DCX, GFAP, or PDGFRa; n=6. Gene expression box plot of (E) DCX, (F) GFAP, and (G) PDGFRa at 35 DPI between SCI+Ctrl and SCI+Gsx1 group. Each dot represents the gene expression as log 2(count per million) for one biological replicate sample. Mean±SEM; *=p<0.05; Students' t-test.

FIGS. 7A-7D: Gsx1 induces neurogenesis at similar level in dorsal and ventral region of the spinal cord tissue. Confocal images of cross-sections (dorsal and ventral) of spinal cord tissues at 14 DPI show the expression of viral reporter RFP and early neuronal marker (A) Doublecortin DCX, (B) astrocyte marker GFAP, and (C) oligodendrocyte progenitor marker PDGFRa. Arrows indicate cell marker+/RFP+ co-labeled cells. Images in the bottom left corner show a higher magnification z-stack view of the area denoted by a dashed white box. Scale bar=20 μm. (D) Schematic and low magnificent view of the spinal cord. Scale bar=100 μm.

FIG. 8A: Functional enrichment of gene ontology (GO) terms for differentially expressed genes (DEGs) at 14 DPI. Enrichment terms for biological process represented as a scatter plot in a two dimensional semantic space using REVIGO. Circle size indicates the log 10(p-value) of the GO terms.

FIGS. 8B-8C. Gsx1 treatment does not change the number of oligodendrocytes after SCI. Hemisection SCI was performed on 8-12 weeks old mice around T10. Immediately after lentivirus injection encoding Ctrl or Gsx1 gene along with RFP reporter. (B) Animals were harvested 56 DPI and sagittal sections are immunostained with oligodendrocyte marker, Olig2. Bottom left of the image includes the higher magnification z-stack view of the area denoted by a dashed white line to indicate co-expression. Scale bar=20 μm. (C) Quantification of Olig2+/RFP+ at 56 DPI. n=6; Mean±SEM; *=p<0.05; Students' t-test.

FIGS. 9A-9F: Gsx1 induces glutamatergic and cholinergic interneurons and decreases GABAergic interneurons. Confocal images of sagittal sections of spinal cord tissues at 56 DPI show the expression of viral reporter RFP and (A) mature neuron marker NeuN, (B) cholinergic neuron marker ChAT, (C) glutamatergic neuron marker vGlut2, and (D) GABAergic neuron marker GABA. Images in the bottom left corner show a higher magnification z-stack view of the area denoted by a dashed white box. Scale bar=20 (E) Quantification of virally transduced cells co-labeled with a cell marker (n=6). (F) RT-qPCR analysis measuring the mRNA level of genes (NeuN or Hrnbp3, vGlut or Slc17a6, and Chat) associated with mature neurons, normalized to the sham group (n=4). Mean±SEM; *=p<0.05; Students' t-test and One-way ANOVA followed by Tukey post-hoc test and Students' t-test.

FIGS. 10A-10I: Attenuated astrogliosis and glial scar formation. The mRNA level of reactive astrocyte marker genes Gfap and Serpina3n in spinal cord tissues at (A) 3 DPI (n=4) and (B) at 35 DPI (n=4) was measured by RT-qPCR. (C) Differentially expressed genes (DEGs) between SCI+Ctrl and SCI+Gsx1 that are associated with reactive astrocytes (RA) (e.g., Mmmp13, Mmp2, Nes, Axin2, Plaur, and Ctnnb1), scar forming astrocytes (SA) (e.g., Slit2 and Sox9) and both with RA and SA (e.g., Gfap and Vim) at 14 DPI and 35 DPI. (D) Gene expression box plot representing the expression of RA and SA associated genes as a log 2(counts per million) at 14 DPI and (E) at 35 DPI. Each dot represents the gene expression as log 2(count per million) for one biological replicate sample. Images of sagittal sections of spinal cord tissues at 56 DPI show the expression of viral reporter RFP, (F) glial scar markers GFAP and (H) chondroitin sulfate proteoglycan (CSPG) marker CS56. Quantification of immunostained area with (G) anti-GFAP and (I) anti-CS56 around the injury site show reduced signals of GFAP and CS56. Scale bar=50 μm, n=4 for Sham and n=6 for SCI+Ctrl and SCI+Gsx1. Mean±SEM; *=p<0.05; One-way ANOVA followed by Tukey post-hoc test. DPI=days post injury.

FIGS. 11A-11G: Improved locomotor functional recovery after SCI. Lateral hemisection SCI was performed on 8-12 weeks old mice around T9-T10 level immediately followed by the injection of lentivirus encoding Gsx1 along with RFP reporter (lenti-Gsx1-RFP). Lentivirus encoding only the reporter RFP was used as a control (lenti-Ctrl-RFP). Locomotor function was assessed by BMS score at least twice weekly up to 56 DPI. (A) Representative images of hindlimb walking status at 56 DPI and (B) the BMS scores of left hindlimb (n>6). (C) RT-qPCR analysis of differentially expressed genes (Ctnna1 and Col6a2) involved in axon guidance at 35 DPI (n=4; Two-way ANOVA analysis followed by post-hoc test). Heatmaps show Gsx1 upregulated the differentially expressed genes involved in (D) Netrin signaling and (E) axonal guidance from RNA-Seq analysis and IPA comparing among 3, 14, and 35 DPI groups (n>3). Genes that (F) promote and (G) inhibit synaptogenesis between Ctrl and Gsx1 treatment at 35 DPI identified using RNA-Seq and IPA analysis (n=4). Mean±SEM *p<0.05, Students' t-test.

FIGS. 12A-12C: Gsx1 treatment promotes signaling for axon growth and 5-HT neuronal activity after hemisection SCI. (A) IPA heat map of differentially expressed genes involved in CREB signaling in neurons at 3 DPI, 14 DPI, and 35 DPI between SCI+Ctrl and SCI+Gsx1; n≥3. (B) Genes involved in the Netrin signaling along with their log 2(fold change) at 35 DPI; n=4. (C) Representative photomicrographs of serotonin (5-HT) staining of the sagittal sections of the spinal cord samples at 56 DPI. “X” indicates lentivirus injection site, and white line indicates hemisection site. n=5; Scale bar=100 μm.

FIG. 13: Functional enrichment of gene ontology (GO) terms for differentially expressed genes (DEGs) at 35 DPI. Enrichment terms for biological process represented as a scatter plot in a two dimensional semantic space using REVIGO. Circle size indicates the log 10(p-value) of the GO terms.

FIG. 14: Effect of GSX1 on various pathways, leading to increased locomotion in a mammal with SCI.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The sequence listing submitted herewith, generated on Jul. 22, 2019, 32 kb, is herein incorporated by reference in its entirety.

SEQ ID NOS: 1-2 are exemplary human Gsx1 coding and protein sequences, respectively.

SEQ ID NOS: 3-4 are exemplary mouse Gsx1 coding and protein sequences, respectively.

SEQ ID NOS: 5-60 are primers used to detect expression of genes shown in Table 2 using RT-qPCR analysis.

SEQ ID NOS: 61-79 are exemplary cell-penetrating peptides.

SEQ ID NOS: 80-81 are exemplary linker peptides.

### DETAILED DESCRIPTION

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, and sequences associated with the GenBank® Accession Numbers listed (as of Aug. 22, 2018) are herein incorporated by reference in their entireties.

In order to facilitate review of the various embodiments of the disclosure, the following explanations of specific terms are provided:

### I. Terms

Administration: To provide or give a subject an agent, such as a Gsx1 nucleic acid molecule or protein (such as a Gsx1-CPP fusion protein), by any effective route. Exemplary routes of administration include, but are not limited to, injection (such as injection into the CNS, for example injection into the spine or brain, for example at or near the site of injury, for example rostral and/or caudal to the injury site). In some examples, administration is an intrathecal injection (e.g., of Gsx1 or Gsx1-CPP nucleic acid molecule or protein) to treat SCI in lumbar/sacral region, a cisterna magna injection (e.g., of Gsx1 or Gsx1-CPP nucleic acid molecule or protein) to treat SCI in cervical/thoracic region, or intraparenchymal or introcerebroventricular injection (e.g., of Gsx1 nucleic acid molecule or protein) to treat traumatic brain injury.

Chimeric or fusion protein: A protein that includes a first peptide (e.g., Gsx1) and a second peptide (e.g., a cell penetrating peptide, such as one or more of those provided in SEQ ID NOS: 61-79), where the first and second proteins are different. A chimeric polypeptide also encompasses polypeptides that include two or more non-contiguous portions derived from the same polypeptide. In some examples, a chimeric protein is a Gsx1-cell penetrating peptide fusion protein (Gsx1-CPP), wherein the Gsx1 portion can have at least 80%, at least 85%, at least 90%, at least 95%, at least 98%, at least 99%, or 100% sequence identity to SEQ ID NO: 2 or 4 and wherein the cell penetrating peptide (CPP) is at the N- or C-terminus of Gsx1. The two or more different peptides can be joined directly or indirectly, for example using a linker (such as 1-30 amino acids).

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

Contact: Placement in direct physical association, including a solid or a liquid form. Contacting can occur in vitro or ex vivo, for example, by adding a reagent to a sample (such as one containing neural cells), or in vivo by administering to a subject.

Effective amount: The amount of an agent (such as a Gsx1 protein, Gsx1-CPP fusion protein, or nucleic acid molecule encoding such) that is sufficient to effect beneficial or desired results.

A therapeutically effective amount may vary depending upon one or more of: the subject and disease condition being treated, the weight and age of the subject, the severity of the disease condition, the manner of administration and the like, which can be determined by one of ordinary skill in the art. The beneficial therapeutic effect can include amelioration of a disease, symptom, disorder, or pathological condition; reducing or preventing the onset of a disease, symptom, disorder or condition; and generally counteracting a disease, symptom, disorder or pathological condition. In one embodiment, an “effective amount” is an amount sufficient to (1) decrease inflammation, for example at or near the injury site, such as decrease the number of infiltrated macrophages (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such), (2) increase the number of neural stem/progenitor cells (NSPCs) (e.g., as determined by measuring expression of nestin and/or Sox2), for example at or near the injury site, (for example an increase of at least 5%, at least 10%, at least 15%, at least 20%, at least 50%, at least 75%, at least 90%, or at least 100%, for example relative to no administration of the Gsx1 protein, Gsx1-CPP fusion protein, or nucleic acid molecule encoding such), (3) increase differentiation of NSPCs towards a neuronal linage, such as an increase in the number of glutamatergic neurons, for example at or near the injury site, (e.g., as determined by measuring expression of NeuN or vGlut2) (for example an increase of at least 5%, at least 10%, at least 15%, at least 20%, at least 50%, at least 75%, at least 90%, or at least 100%, at least 200%, at least 300%, or at least 600%, for example relative to no administration of the Gsx1 protein , Gsx1-CPP fusion protein, or nucleic acid molecule encoding such), (4) decrease astrogliosis and glial scar formation, for example at or near the injury site, such as decreasing the number of astrocytes (e.g., as determined by measuring expression of GFAP or CSPG) (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such), (5) increase locomotion of the subject (e.g., as determined by Basso Mouse Scale (BMS) score, Functional Independence Measure (FIM) score (Functional Independence Measure: Guide for the Uniform Data Set for Medical Rehabilitation (Adult FIM), Version 4.0 . Buffalo, N.Y.: State University of New York at Buffalo, 1993), or the motor score of the American Spinal Injury Association (ASIA)) (for example an increase of at least 5%, at least 10%, at least 15%, at least 20%, at least 50%, at least 75%, at least 90%, or at least 100%, at least 200%, at least 300%, or at least 600%, for example relative to no administration of the Gsx1 protein , Gsx1-CPP fusion protein, or nucleic acid molecule encoding such), and/or (6) decrease cell death, for example at or near the injury site, such as decreasing the number of cleaved caspase3 positive cells (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such).

Expression: The process by which the coded information of a nucleic acid molecule, such as a Gsx1 nucleic acid molecule is converted into an operational, non-operational, or structural part of a cell, such as the synthesis of a protein (e.g., Gsx1 protein or Gsx1-CPP fusion protein). Expression of a gene can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule or protein can be altered relative to a normal (wild type) nucleic acid molecule or protein (such as in a normal non-recombinant cell). Alterations in gene expression, such as differential expression, include but are not limited to: (1) overexpression (e.g., upregulation); (2) underexpression (e.g., downregulation); or (3) suppression of expression. Alternations in the expression of a nucleic acid molecule can be associated with, and in fact cause, a change in expression of the corresponding protein.

Genomic Screened Homeo Box (GSX1): (e.g., OMIM 616542): Also known as Gsh1. This gene encodes a protein involved in pituitary development. The mouse protein is 261 amino acids, and the human protein is 264 amino acids, and the two proteins share about 96% sequence homology. The human GSX1 gene maps to chromosome 13q12.2. Gsx1 sequences are publically available, for example from the GenBank® sequence database (e.g., Accession Nos. NP_663632.1, NP_032204.1, XP_006068096.2, and NP_001178592.1 provide exemplary Gsx1 protein sequences, while Accession Nos. NM_145657.2, NM_008178.2, XM_006068034.2, and NM_001191663.1 provide exemplary Gsx1 nucleic acid sequences). One of ordinary skill in the art can identify additional Gsx1 nucleic acid and protein sequences, including Gsx1 variants, such as those having at least 80%, at least 85%, at least 90%, at least 92%, at least 95%, at least 98%, or at least 99% sequence identity to these GenBank® sequences (such as to SEQ ID NO: 1, 2, 3 or 4). Such Gsx1 sequences can be used to generate therapeutic recombinant nucleic acid molecules and proteins, for example to treat a neurological disorder, such as a SCI, using the methods provided herein.

In one example, a Gsx1 protein is part of a fusion protein, such as a Gsx1-CPP fusion protein.

Increase or Decrease: A statistically significant positive or negative change, respectively, in quantity from a control value. An increase is a positive change, such as an increase at least 50%, at least 100%, at least 200%, at least 300%, at least 400% or at least 500% as compared to the control value. A decrease is a negative change, such as a decrease of at least 20%, at least 25%, at least 50%, at least 75%, at least 80%, at least 90%, at least 95%, at least 98%, at least 99%, or at least 100% decrease as compared to a control value. In some examples the decrease is less than 100%, such as a decrease of no more than 90%, no more than 95% or no more than 99%.

Isolated: An “isolated” biological component (such as a protein or nucleic acid, or cell) has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins (such as Gsx1 proteins and nucleic acid molecules) prepared by recombinant expression in a host cell as well as chemically synthesized nucleic acids and proteins. Isolated proteins, nucleic acids, or cells in some examples are at least 50% pure, such as at least 75%, at least 80%, at least 90%, at least 95%, at least 98%, or at least 100% pure.

Linker: A moiety or group of moieties that joins or connects two or more discrete separate peptide or proteins, such as monomer domains, for example to generate a chimeric protein. In one example a linker is a substantially linear moiety. Exemplary linkers that can be used to generate the chimeric proteins provided herein include but are not limited to: peptides, nucleic acid molecules, peptide nucleic acids, and optionally substituted alkylene moieties that have one or more oxygen atoms incorporated in the carbon backbone. A linker can be a portion of a native sequence, a variant thereof, or a synthetic sequence. Linkers can include naturally occurring amino acids, non-naturally occurring amino acids, or a combination of both. In one example a linker is composed of at least 5, at least 10, at least 15 or at least 20 amino acids, such as 5 to 10, 5 to 20, or 5 to 50 amino acids. In one example the linker is a polyalanine. In one example the linker is a flexible linker, such as one that includes Gly and Ser residues (e.g., GSGSGS (SEQ ID NO: 80) or GGSGGGGSGG, SEQ ID NO: 81).

Non-naturally occurring or engineered: Terms used herein as interchangeably and indicate the involvement of the hand of man. The terms, when referring to nucleic acid molecules or polypeptides indicate that the nucleic acid molecule or the polypeptide is at least substantially free from at least one other component with which they are naturally associated in nature and as found in nature. In addition, the terms can indicate that the nucleic acid molecules or polypeptides is one having a sequence not found in nature.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence (such as a Gsx1 coding sequence) if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of recombinant nucleic acid molecule or protein (such as Gsx1 or Gsx1-CPP fusion protein).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide, peptide and protein: Refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

Promoter: An array of nucleic acid control sequences which direct transcription of a nucleic acid, such as a Gsx1 or Gsx1-CPP fusion protein coding sequence. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. In contrast, the activity of an “inducible promoter” is regulated by an external signal or molecule (for example, a transcription factor). In one example the promoter used is native to the nucleic acid molecule it is expressing (endogenous promoter), for example, is endogenous to Gsx1. In one example the promoter used is not native to the nucleic acid molecule it is expressing (exogenous promoter). A “tissue-specific promoter” is a promoter that direct expression of a nucleic acid molecule in particular cells or tissues, such as the central nervous system. Exemplary promoters that can be used to drive expression of Gsx1 include: CMV promoter, SV40 promoter, beta actin promoter, or inducible Tetracycline (Tet) inducible lentiviral system (Tet on or off system).

Recombinant or host cell: A cell that has been genetically altered, or is capable of being genetically altered by introduction of an exogenous polynucleotide, such as a recombinant plasmid or vector. Typically, a host cell is a cell in which a recombinant nucleic acid molecule can be propagated and/or its DNA expressed. Such cells can be a neural cell. The term also includes any progeny of the subject host cell. It is understood that all progeny may not be identical to the parental cell since there may be mutations that occur during replication. However, such progeny are included when the term “host cell” is used.

Regulatory element: Includes promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990) which is hereby incorporated by reference in its entirety. Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as neural tissues or cells. Regulatory elements may also direct expression in a temporal-dependent manner, such as in a cell-cycle dependent or developmental stage-dependent manner, which may or may not also be tissue or cell-type specific.

In some embodiments, a Gsx1 or Gsx1-CPP coding sequence is operably linked to a promoter, such as a constitutive promoter, such as a pol III promoter, pol II promoter, or pol I promoter. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, CAG promoter, UBC promoter, ROSA promoter, and the EF1α promoter. In some embodiments, a Gsx1 coding sequence is operably linked to a tissue-specific promoter, such as a CNS-specific promoter.

Also encompassed by the term “regulatory element” are enhancer elements, such as WPRE; CMV enhancers; the R-U5′ segment in LTR of HTLV-I (Mol. Cell. Biol., Vol. 8(1):466-472, 1988); SV40 enhancer; and the intron sequence between exons 2 and 3 of rabbit β-globin (Proc. Natl. Acad. Sci. USA., 78(3):1527-31, 1981). In some embodiments, a Gsx1 coding sequence is operably linked to an enhancer, such as a neural-specific enhancer (e.g., Notch1CR2 or Olig2CR5).

In some embodiments, a Gsx1 or Gsx1-CPP coding sequence is operably linked to both a promoter and an enhancer, such as a constitutive promoter (e.g., CMV) and a neural-specific enhancer (e.g., Notch1CR2 or Olig2CR5).

Sequence identity/similarity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of protein and nucleic acid sequences (including the Gsx1 sequences provided herein) are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: A mammal, such as a human or veterinary subject. Mammals include, but are not limited to, murines, simians, humans, farm animals, sport animals, and pets. In one embodiment, the subject is a non-human mammalian subject, such as a monkey or other non-human primate, mouse, rat, rabbit, pig, goat, sheep, dog, cat, boar, bull, horse, or cow. In some examples, the subject is a laboratory animal/organism, such as a mouse, rabbit, or rat. In some examples, the subject has a neurological disorder, such as a neurodegenerative disease or has suffered a traumatic brain injury or traumatic SCI that can be treated using the methods provided herein.

Therapeutic agent: Refers to one or more molecules or compounds that confer some beneficial effect upon administration to a subject. The beneficial therapeutic effect can include enablement of diagnostic determinations; amelioration of a disease, symptom, disorder, or pathological condition; reducing the onset of a disease, symptom, disorder or condition; and generally counteracting a disease, symptom, disorder or pathological condition, such as a neurological disorder.

Transduced, Transformed, Transfected: A virus or vector “transduces” a cell when it transfers nucleic acid molecules into a cell. A cell is “transformed” or “transfected” by a nucleic acid transduced into the cell when the nucleic acid becomes stably replicated by the cell, either by incorporation of the nucleic acid into the cellular genome, or by episomal replication.

These terms encompass all techniques by which a nucleic acid molecule can be introduced into such a cell, including transfection with viral vectors, transformation with plasmid vectors, and introduction of naked DNA by electroporation, lipofection, particle gun acceleration and other methods in the art. In some example the method is a chemical method (e.g., calcium-phosphate transfection or polyethyleneimine (PEI) transfection), physical method (e.g., electroporation, microinjection, particle bombardment), fusion (e.g., liposomes), receptor-mediated endocytosis (e.g., DNA-protein complexes, viral envelope/capsid-DNA complexes) and biological infection by viruses such as recombinant viruses (Wolff, J. A., ed, Gene Therapeutics, Birkhauser, Boston, USA, 1994). Methods for the introduction of nucleic acid molecules into cells are known (e.g., see U.S. Pat. No. 6,110,743).

Transgene: An exogenous gene, for example supplied by a vector (such as a viral vector). In one example, a transgene includes a Gsx1 or Gsx1-CPP coding sequence, for example operably linked to a promoter sequence.

Transgenic: A cell or animal (e.g., human or mouse) carrying a transgene.

Treating, Treatment, and Therapy: Any success or indicia of success in the attenuation or amelioration of a pathology or condition, including any objective or subjective parameter such as abatement or diminishing of symptoms. The treatment may be assessed by objective or subjective parameters; including the results of a physical examination, and other clinical tests, and the like. In one example, treatment using the disclosed methods (1) decreases inflammation, for example at or near the injury site, such as decrease the number of infiltrated macrophages (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such), (2) increases the number of neural stem/progenitor cells (NSPCs) (e.g., as determined by measuring expression of nestin and/or Sox2), for example at or near the injury site, (for example an increase of at least 5%, at least 10%, at least 15%, at least 20%, at least 50%, at least 75%, at least 90%, or at least 100%, for example relative to no administration of the Gsx1 protein, Gsx1-CPP fusion protein, or nucleic acid molecule encoding such), (3) increases differentiation of NSPCs towards a neuronal linage, such as an increase in the number of glutamatergic neurons, for example at or near the injury site, (e.g., as determined by measuring expression of NeuN or Glut2) (for example an increase of at least 5%, at least 10%, at least 15%, at least 20%, at least 50%, at least 75%, at least 90%, or at least 100%, at least 200%, at least 300%, or at least 600%, for example relative to no administration of the Gsx1 protein, Gsx1-CPP fusion protein, or nucleic acid molecule encoding such), (4) decreases astrogliosis and glial scar formation, for example at or near the injury site, such as decreasing the number of astrocytes (e.g., as determined by measuring expression of GFAP or CSPG) (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such), (5) increases locomotion of the subject (e.g., as determined by Basso Mouse Scale (BMS) score or Functional Independence Measure (FIM) score) (for example an increase of at least 5%, at least 10%, at least 15%, at least 20%, at least 50%, at least 75%, at least 90%, or at least 100%, at least 200%, at least 300%, or at least 600%, for example relative to no administration of the Gsx1 protein, Gsx1-CPP fusion protein, or nucleic acid molecule encoding such), and/or (6) decreases cell death, for example at or near the injury site, such as decreasing the number of cleaved caspase3 positive cells (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such).

Upregulated: When used in reference to the expression of a molecule, such as a gene or a protein (e.g., Gsx1), refers to any process which results in an increase in production of a gene product. A gene product can be RNA (such as mRNA, rRNA, tRNA, and structural RNA) or protein. Therefore, upregulation includes processes that increase transcription of a gene or translation of mRNA and thus increase the presence of proteins or nucleic acids. The disclosed methods, can be used to upregulate Gsx1.

Examples of processes that increase transcription include those that increase transcription initiation rate, those that increase transcription elongation rate, those that increase processivity of transcription and those that decrease transcriptional repression. Gene upregulation can include increasing expression above an existing level. Examples of processes that increase translation include those that increase translational initiation, those that increase translational elongation and those that increase mRNA stability.

Upregulation includes any detectable increase in the production of a gene product. In certain examples, detectable Gsx1 protein or nucleic acid expression in a cell (such as a cell of the CNS) increases by at least 20%, at least 30%, at least 40%, at least 50%, at least 60%, at least 70%, at least 75%, at least 80%, at least 90%, at least 95%, at least 100%, at least 200%, at least 400%, or at least 500% as compared to a control (such an amount of protein or nucleic acid expression detected in a corresponding normal or non-recombinant cell). In one example, a control is a relative amount of expression in a normal cell (e.g., a non-recombinant CNS cell, such as a neural cell).

Under conditions sufficient for: A phrase that is used to describe any environment that permits a desired activity. In one example the desired activity is expression of a Gsx1 nucleic acid molecule to treat a neurological disorder.

Vector: A nucleic acid molecule into which a foreign nucleic acid molecule can be introduced without disrupting the ability of the vector to replicate and/or integrate in a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art.

A vector can include nucleic acid sequences that permit it to replicate in a host cell, such as an origin of replication. A vector can also include one or more selectable marker genes (such as antibiotic resistance or a fluorescent protein), and other genetic elements. An integrating vector is capable of integrating itself into a host nucleic acid. An expression vector is a vector that contains the regulatory sequences to allow transcription and translation of inserted gene or genes.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as 3rd generation integration-deficient lentiviral vectors) or adeno-associated viral (AAV) vector.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

Certain vectors are capable of directing the expression of genes to which they are operatively-linked, such as a Gsx1 or Gsx1-CPP coding sequence. Such vectors are referred to herein as “expression vectors.” Common expression vectors of utility in recombinant DNA techniques are often in the form of plasmids. Recombinant expression vectors can include a nucleic acid provided herein (such as a Gsx1 or Gsx1-CPP coding sequence) in a form suitable for expression of the nucleic acid in a host cell, which means that the recombinant expression vectors can include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). The design of the expression vector can depend on such factors as the choice of the host cell to be transformed, the level of expression desired, the size of the transgenic cargo, etc. A vector can be introduced into host cells to thereby produce transcripts, proteins, or peptides, including fusion proteins or peptides, encoded by nucleic acids as described herein (e.g., Gsx1 or Gsx1-CPP).

### II. Overview

Limited neurogenesis, increased reactive astrogliosis, and scar formation are the major barriers for neuroregeneration and functional recovery after SCI. It is shown herein that Gsx1 treatment at or near the injury site promotes the activation of NSPCs and the generation of specific subtypes of interneurons (e.g., glutamatergic and cholinergic neurons). Gsx1 expression also inhibits reactive astrogliosis and glial scar formation, and leads to a dramatic locomotor functional recovery in mice with lateral hemisection SCI. The RNA-Seq and RT-qPCR analysis demonstrates that Gsx1 treatment alters the expression of genes associated with cell proliferation, NSPC activation, neurogenesis, astrogliosis, and scar formation, which correlates with functional recovery after SCI.

Previous studies using transcription factors, e.g., Sox2 and NeuroD1 have shown their successful induction of neurons. However, limited or no functional recovery have been reported. The failure of newly generated neurons for functional recovery may be attributed to the following aspects: 1) Sox2 and NeuroD1 are general neurogenic transcription factors, but not specific transcription factors for spinal neuronal genesis; 2) Sox2-induced neurons resemble GABAergic interneurons. The additional inhibitory interneurons might have caused a further imbalance of the excitation/inhibition ratio; and 3) functional recovery may require the generation of various specific cell types, e.g., glutamatergic and cholinergic interneurons. Spinal inhibitory interneurons act as a roadblock limiting the integration of descending inputs into relay circuits after injury. It is shown herein that Gsx1 treatment inhibits the generation of GABAergic interneurons. Thus, Gsx1 treatment-induced reduction of GABAergic interneurons may contribute to the restoration of the excitation/inhibition ratio.

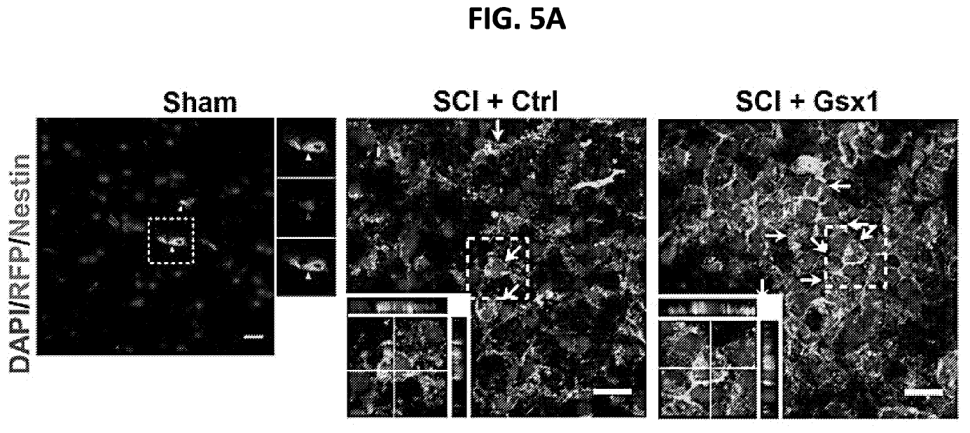

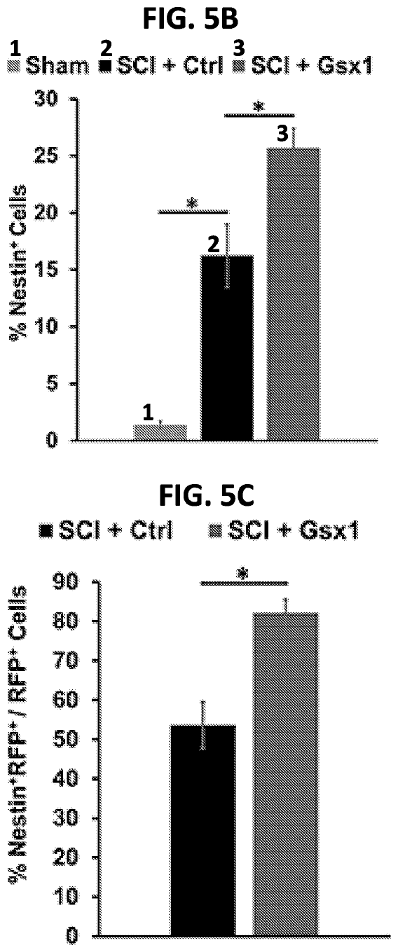

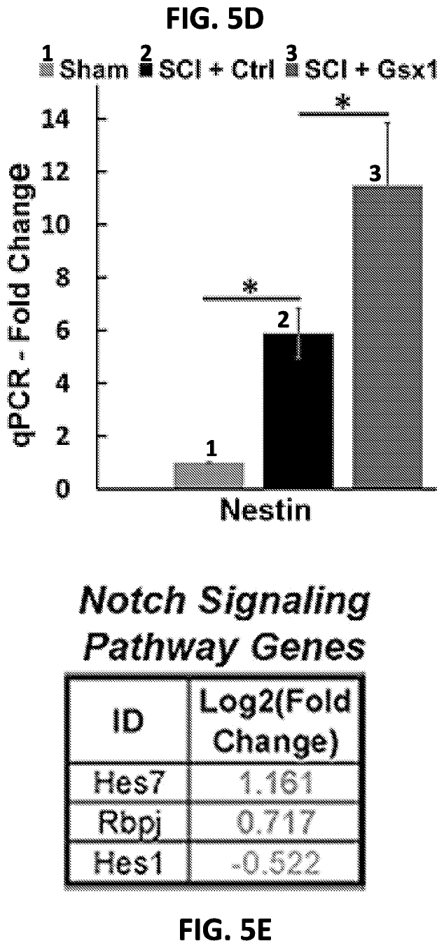

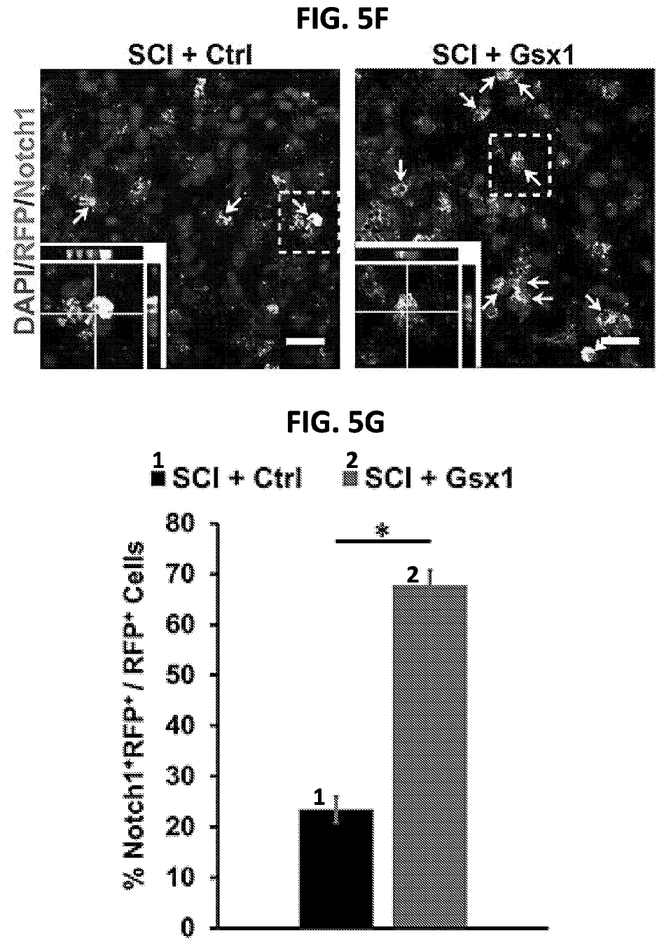

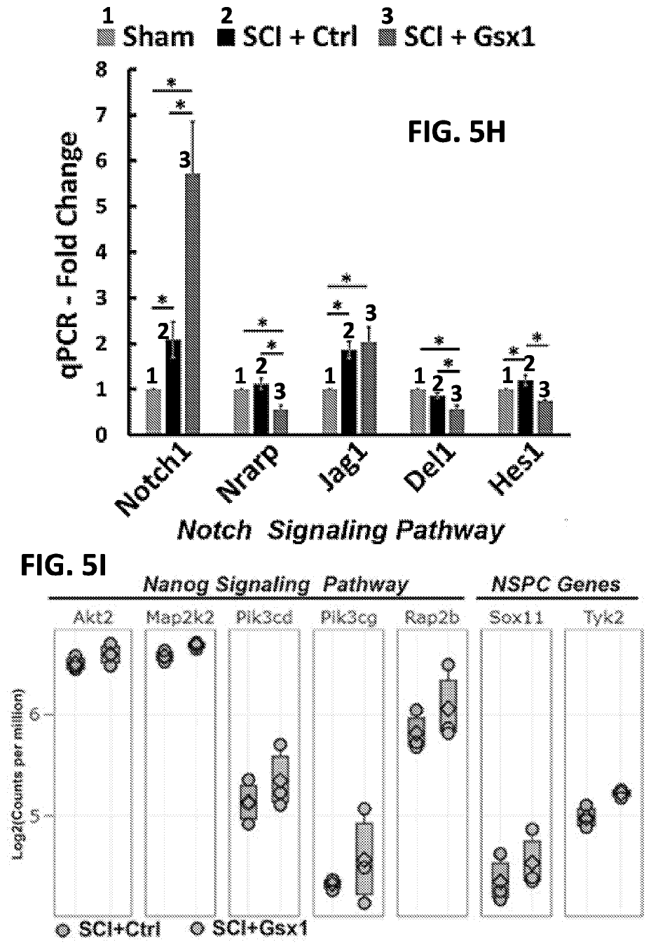

Gsx1 regulates Notch signaling via its interaction with a Notch1 enhancer. At the embryonic stage, an increase in Gsx1 and Notch1 leads to a higher level of glutamate neurotransmitters. Notch signaling is a canonical pathway required for NSPC proliferation and self-renewal, as well as for prevention of untimely neuronal differentiation of NSPCs. The RNA-Seq and RT-qPCR data herein show that Gsx1 transiently upregulates Notch and Nanog signaling pathways during an acute stage of SCI (FIGS. 5E-5G).

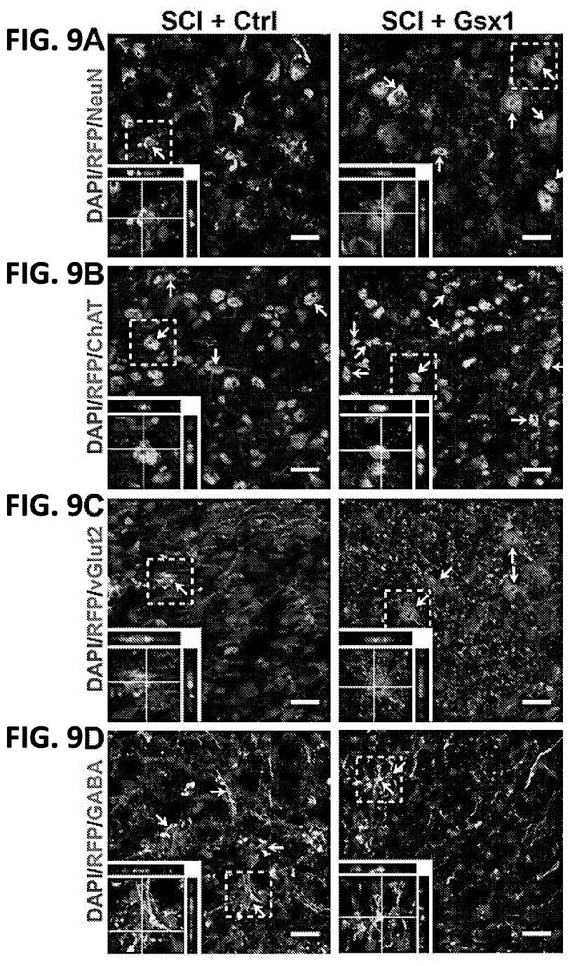

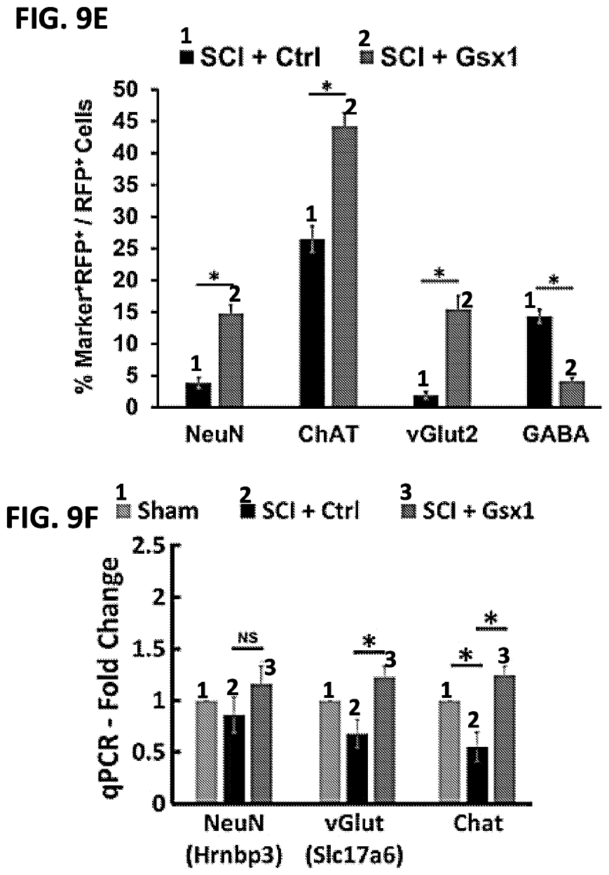

Gsx1 treatment increased the number of glutamatergic and cholinergic neurons and decreased the GABAergic interneurons at the injury site (FIGS. 9A-9F). These upregulated signaling pathways (FIGS. 5A-5I) support the activation and expansion of endogenous NSPCs.

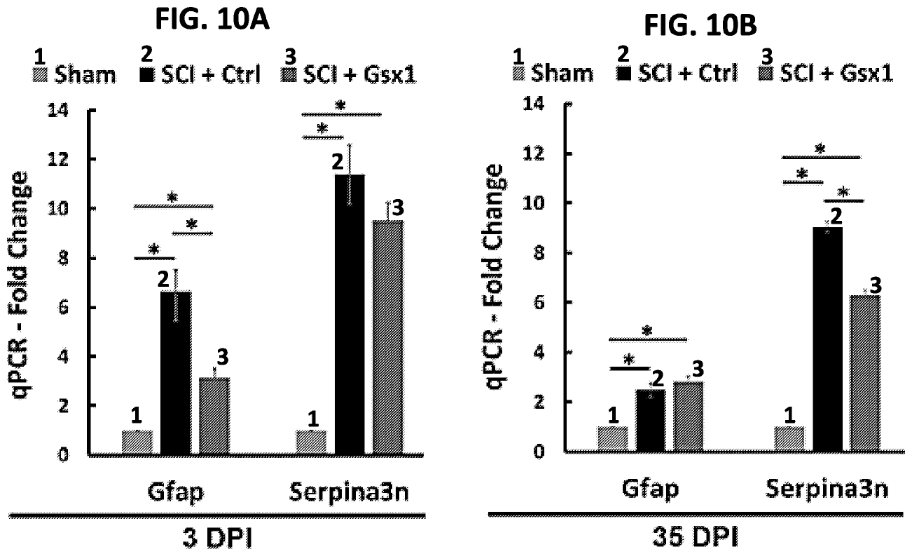

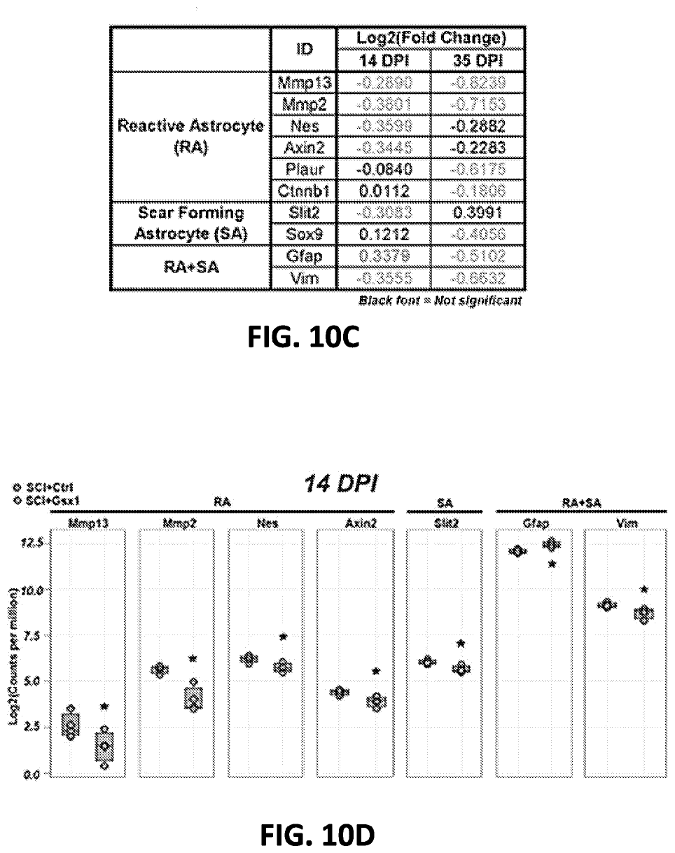

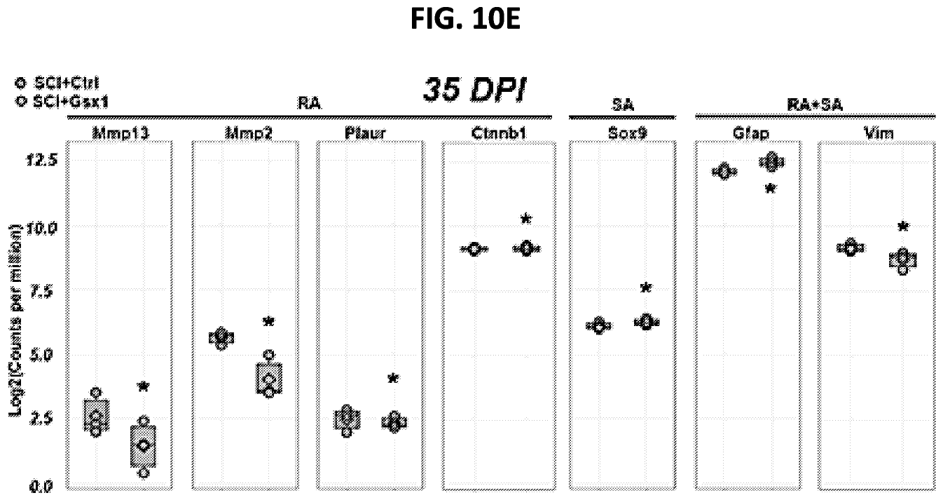

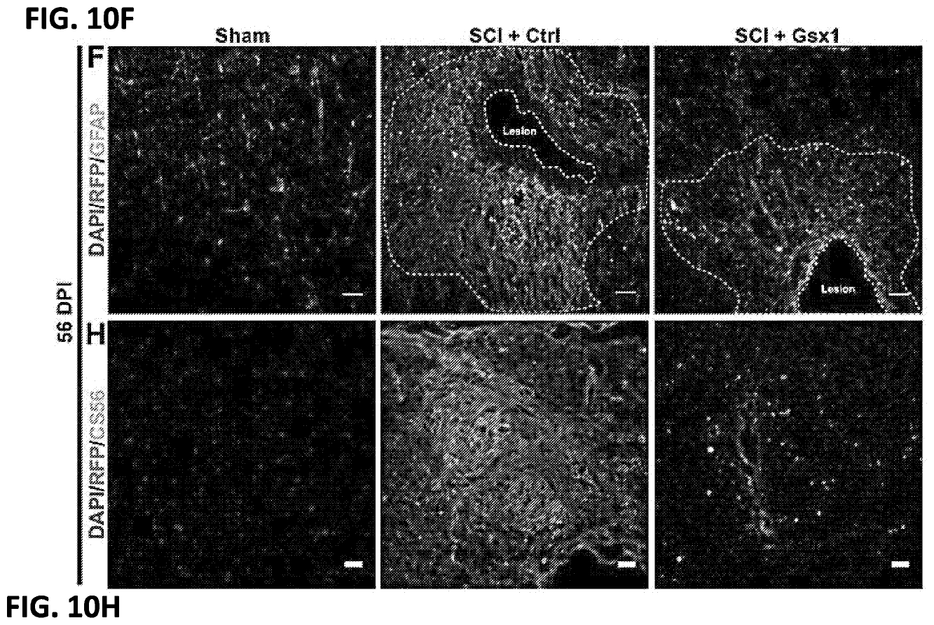

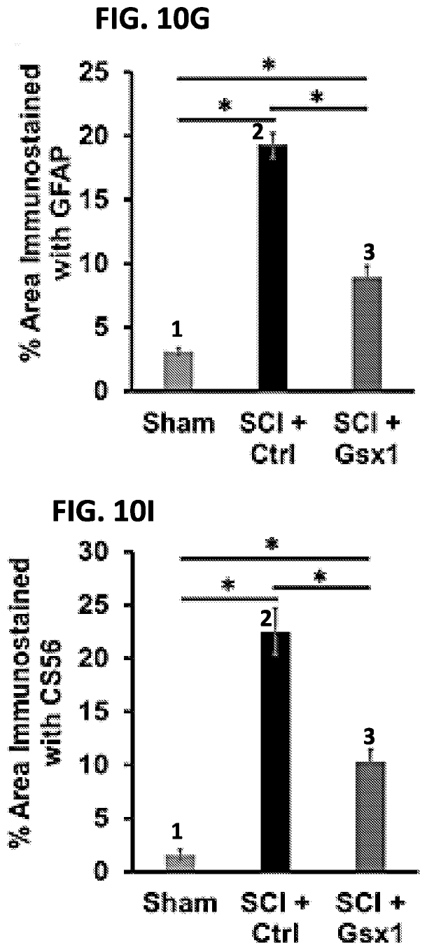

The observation that Gsx1 treatment reduces reactive astrogliosis and scar formation is consistent with functional recovery and such a role for Gsx1 has not been previously reported. In fact, the adult NSPCs give rise to mostly astrocytes after CNS injury (Benner et al., Nature 497:369-373 (2013)). However, Gsx1 treatment significantly decreases in the expression of genes associated with reactive astrocytes (RA) and scar forming astrocytes (SA). Gsx1-induced NSPC differentiation into neuronal lineage may be at the expense of the astrocyte lineage. Reduction in astrogliosis leads to attenuation of scar formation (FIGS. 10A-10I).

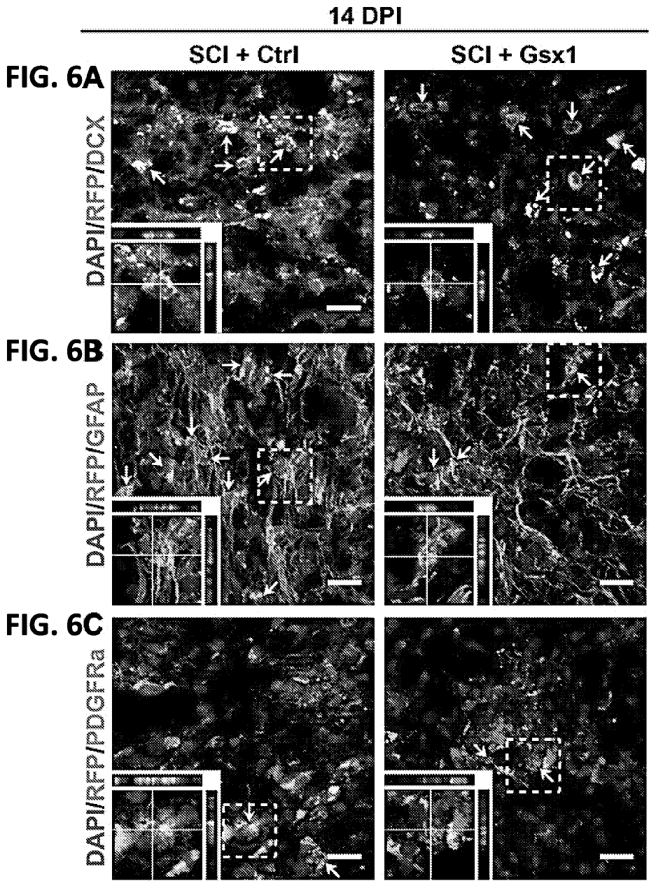

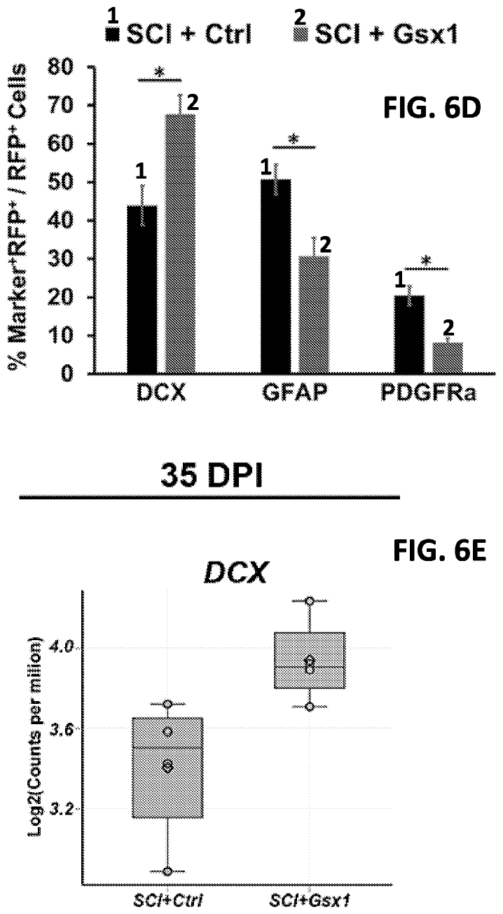

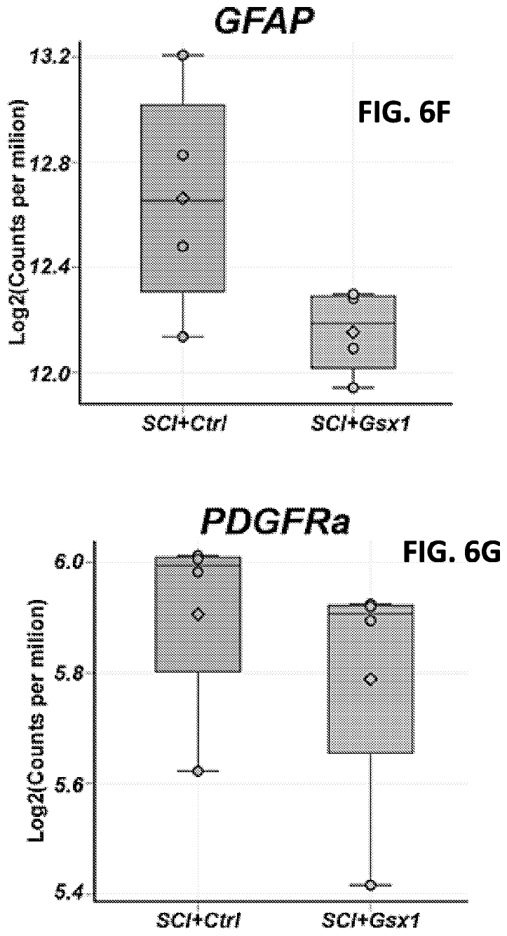

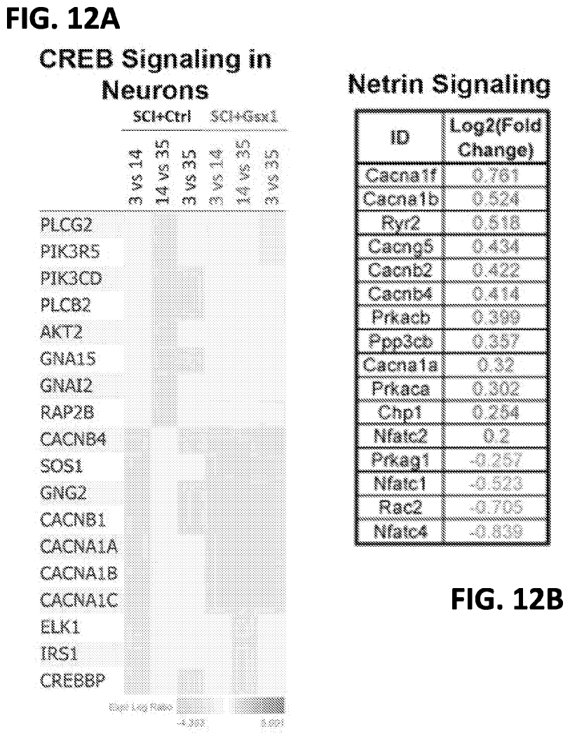

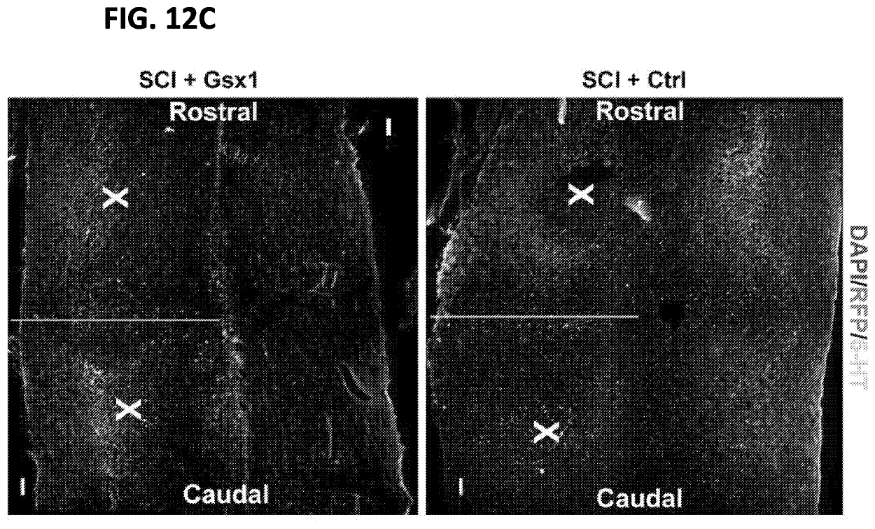

For Gsx1-induced neurons to be functional, they need to establish proper connections. The methods provided herein upregulate axon guidance signaling, Netrin signaling, CREB signaling pathway, and synaptogenesis (FIGS. 6A-6G, 12A, 12B).

Based on these observations, methods are provided for expressing Gsx1 in the injured spinal cord to (i) reduce glial scar (such as a reduction of at least 20%, at least 25%, or at least 30%, for example relative to no administration of a Gsx1 protein, Gsx1-CPP fusion protein, or nucleic acid molecule encoding such), (ii) induce neurogenesis (such as an increase in neurogenesis of at least 20%, at least 40%, or at least 50%, for example relative to no administration of a Gsx1 protein, Gsx1-CPP fusion protein, or nucleic acid molecule encoding such), and/or (iii) improve locomotion after SCI (such as an increase in locomotion of at least 50%, at least 100%, at least 200%, or at least 300%, for example relative to no administration of a Gsx1 protein, Gsx1-CPP fusion protein, or nucleic acid molecule encoding such). The ability of Gsx1 treatment to not only successfully produce specific types of mature neurons and reduced glial scar, but also significant locomotor functional recovery in mice with SCI demonstrates that Gsx1 is a therapeutic agent for the treatment of SCI and other central nervous related injuries (FIG. 14).

### III. Methods of Treatment

Provided herein are methods for treating a neurological disorder in a mammalian subject, such as a human or veterinary subject. The methods can include administering to the subject a therapeutically effective amount of Gsx1 protein, Gsx1-CPP fusion protein, or a nucleic acid molecule encoding Gsx1 or Gsx1-CPP, thereby treating the neurological disorder. In one example, the administration is via injection, such as injection into the CNS (e.g., spinal cord or brain). For example, the Gsx1 protein, Gsx1-CPP fusion protein, or a nucleic acid molecule encoding Gsx1 or Gsx1-CPP, can be administered near or at the site of the brain or spinal cord injury, such as rostral and/or caudal to the injury site.

In one example, a Gsx1 protein is administered that has at least 80%, at least 85%, at least 90%, at least 95%, at least 98%, at least 99%, or 100% sequence identity to SEQ ID NO: 2 or 4. In another example, a Gsx1-CPP fusion protein comprising a Gsx1 protein and a cell penetrating peptide is administered, wherein the Gsx1 portion of the fusion protein has at least 80%, at least 85%, at least 90%, at least 95%, at least 98%, at least 99%, or 100% sequence identity to SEQ ID NO: 2 or 4. In some examples, the CPP domain of the Gsx1-CPP fusion protein has at least 80%, at least 85%, at least 90%, at least 95%, at least 98%, at least 99%, or 100% sequence identity to any one of SEQ ID NOS: 61-79. Thus, in some examples, a Gsx1 portion of a Gsx1-CPP fusion protein has at least 80%, at least 85%, at least 90%, at least 95%, at least 98%, at least 99%, or 100% sequence identity to SEQ ID NO: 2 or 4, and the CPP domain of the Gsx1-CPP fusion protein has at least 80%, at least 85%, at least 90%, at least 95%, at least 98%, at least 99%, or 100% sequence identity to any one of SEQ ID NOS: 61-79. In some examples, the CPP domain of the Gsx1-CPP fusion protein is C-terminal to the Gsx1 domain. In some examples, the CPP domain of the Gsx1-CPP fusion protein is N-terminal to the Gsx1 domain. In some examples, the CPP domain of the Gsx1-CPP fusion protein is directly linked to the Gsx1 domain. In some examples, the CPP domain of the Gsx1-CPP fusion protein is indirectly linked to the Gsx1 domain, for example via a peptide linker, such as a linker of 1 to 30 amino acids, such as a linker having at least 80%, at least 85%, at least 90%, at least 95%, at least 98%, at least 99%, or 100% sequence identity to SEQ ID NO: 80 or 81.

In another example, a Gsx1 encoding nucleic acid molecule is administered, wherein the nucleic acid molecule encoding Gsx1 encodes a protein comprising at least 80%, at least 85%, at least 90%, at least 95%, at least 98%, at least 99%, or 100% sequence identity to SEQ ID NO: 2 or 4. In some examples, the nucleic acid molecule encoding Gsx1 includes at least 80%, at least 85%, at least 90%, at least 95%, at least 98%, at least 99%, or 100% sequence identity to SEQ ID NO: 1 or 3. In one example, a Gsx1-CPP encoding nucleic acid molecule is administered, wherein the nucleic acid molecule encoding Gsx1-CPP includes a portion that encodes a Gsx1 domain having at least 80%, at least 85%, at least 90%, at least 95%, at least 98%, at least 99%, or 100% sequence identity to SEQ ID NO: 2 or 4. In some examples, the nucleic acid molecule encoding Gsx1-CPP includes a portion that encodes a CPP domain having at least 80%, at least 85%, at least 90%, at least 95%, at least 98%, at least 99%, or 100% sequence identity to any one of SEQ ID NOS: 61 to 79. In some examples, the nucleic acid molecule encoding the Gsx1 domain of Gsx1-CP includes at least 80%, at least 85%, at least 90%, at least 95%, at least 98%, at least 99%, or 100% sequence identity to SEQ ID NO: 1 or 3.

Such Gsx1 and Gsx1-CPP coding sequences can include other elements. In one example, the nucleic acid molecule encoding Gsx1 or Gsx1-CPP is part of a plasmid or viral vector, such as a lentiviral vector or adeno-associated viral vector. In some examples, the nucleic acid molecule encoding Gsx1 or Gsx1-CPP is operably linked to a promoter, such as a constitutive promoter (e.g., CMV, beta actin, or a native Gsx1 promoter), or a tissue-specific promoter, such as a central nervous system (CNS)-specific promoter (e.g., a synapasin 1 (Syn1) promoter, glial fibrillary acidic protein (GFAP) promoter, nestin (NES) promoter, myelin-associated oligodendrocyte basic protein (MOBP) promoter, myelin basic protein (MBP) promoter, tyrosine hydroxylase (TH) promoter, or a forkhead box A2 (FOXA2) promoter). In some examples, the nucleic acid molecule encoding a Gsx1-CPP fusion protein is operably linked to a promoter, such as a constitutive promoter (e.g., CMV, beta actin, or a native Gsx1 promoter), wherein the Gsx1 portion of the Gsx1-CPP fusion protein has at least 80%, at least 85%, at least 90%, at least 95%, at least 98%, at least 99%, or 100% sequence identity to SEQ ID NO: 2 or 4.

Exemplary neurological disorders that can be treated with the disclosed methods include spinal cord injuries, brain injuries, or both. In one example, the spinal cord injury, brain injury, or both is caused by trauma from an external force, such as a blow or jolt to the head or a penetrating head injury, such as a vehicle crash (e.g., car, motorcycle, ATV, or bike), fall, act of violence (e.g., gun-shot wound or stab wound), or sports (e.g., a collision or fall resulting during football, soccer, baseball, hockey, diving, skiing, rugby, lacrosse, horseback riding, or basketball). A spinal cord injury usually begins with a sudden, traumatic blow to the spine that fractures or dislocates vertebrae. Most injuries to the spine do not completely sever it, but instead cause fracture or compressions of the vertebrae, which then crush and destroy the axons that carry signals up and down the spinal cord. The spinal cord injury can be at the cervical, thoracic, lumbar, sacral, or coccyx region of the spine, such as a C4, C6, T6, T9, T10, or L1 injury. Thus, in some examples, the subject treated with the disclosed methods has quadriplegia or paraplegia.

In one example, the neurological disorder is a traumatic brain injury (TBI), which occurs due to a sudden acceleration or deceleration with the cranium or a combination of movement and sudden impact. Damage occurs both at the time of injury, as well as minutes to days later, for example, due to changes in blood flow and pressure within the cranium. TBI is classified from mild (including concussion) to severe. In some examples, the neurological disorder that can be treated with the disclosed methods is a neurodegenerative disorder, such as Parkinson's disease, Alzheimer's disease, stroke, ischemia, epilepsy, Huntington's disease, multiple sclerosis, or amyotrophic lateral sclerosis. Such neurodegenerative disorders are an abnormality in the nervous system of a mammalian subject, in which neuronal integrity is threatened, for example when neuronal cells display decreased survival or when the neurons can no longer propagate a signal.

In one example, the therapeutically effective amount of Gsx1 protein, Gsx1-CPP fusion protein, or nucleic acid molecule encoding such, is present in a pharmaceutical composition, such as one that includes a pharmaceutically acceptable carrier, such as saline or water.

In some examples, only a single dose of the Gsx1 protein, Gsx1-CPP fusion protein, or nucleic acid molecule encoding such is administered. However, in other examples, the method includes least two separate administrations of the therapeutically effective amount of Gsx1 protein, Gsx1-CPP fusion protein, or nucleic acid molecule encoding such, such as at least 3, at least 4, at least 5, at least 10, or at least 20 separate administrations. In some examples, the at least two separate administrations are separated by at least 1 week, at least 2 weeks, at least 3 weeks, at least 4 weeks, at least 1 month, at least 2 months, at least 3 months, at least 6 months, at least 9 months, or at least one year.

In some examples, administering, the therapeutically effective amount of Gsx1 protein, Gsx1-CPP fusion protein, or nucleic acid molecule encoding such (e.g., as part of a viral vector) occurs within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the neurological disorder.

The disclosed methods can further include administering to the subject a therapeutically effective amount of another neurological disorder therapeutic agent.

In some examples, the method includes selecting a subject with a neurological disorder, such as a traumatic spinal cord or brain injury, or a neurodegenerative disease. These subjects can be selected for treatment with a Gsx1 protein, Gsx1-CPP fusion protein, or nucleic acid molecule encoding such.

In some examples, treating a neurological disorder using the disclosed methods includes one or more of (1) decreasing inflammation, for example at or near the injury site, such as decreasing the number of infiltrated macrophages (such as a decrease of at least 5%, at least 10%, at least 15%, at least 20%, at least 50%, at least 75%, or at least 90% for example relative to no administration of the Gsx1 protein, Gsx1-CPP protein, or nucleic acid molecule encoding such), (2) increasing the number of neural stem/progenitor cells (NSPCs) (e.g., as determined by measuring expression of nestin, Ki67, and/or Sox2), for example at or near the injury site, (for example an increase of at least 5%, at least 10%, at least 15%, at least 20%, at least 50%, at least 75%, at least 90%, at least 100%, at least 200%, at least 300%, or at least 500%, for example relative to no administration of the Gsx1 protein, Gsx1-CPP protein, or nucleic acid molecule encoding such), (3) increasing differentiation of NSPCs towards a specific neuronal linage, such as an increase in the number of glutamatergic neurons, increase in the number of cholinergic neurons (e.g., as determined by measuring expression of NeuN, ChAT, and/or Glut2) (for example an increase of at least 5%, at least 10%, at least 15%, at least 20%, at least 50%, at least 75%, at least 90%, at least 100%, at least 200%, at least 300%, or at least 600%, for example relative to no administration of the Gsx1 protein, Gsx1-CPP protein, or nucleic acid molecule encoding such), decrease the number of GABAergic interneurons (e.g., as determined by measuring expression of GABA) (such as a decrease of at least 5%, at least 10%, at least 15%, at least 20%, at least 50%, at least 75%, or at least 90% for example relative to no administration of the Gsx1 protein, Gsx1-CPP protein, or nucleic acid molecule encoding such), decrease the number of GABAergic interneurons, or combinations thereof, for example at or near the injury site, (4) decreasing astrogliosis and glial scar formation, for example at or near the injury site, such as decreasing the number of astrocytes (e.g., as determined by measuring expression of GFAP, Serpina3n, and/or CSPG) (such as a decrease of at least 5%, at least 10%, at least 15%, at least 20%, at least 50%, at least 75%, or at least 90% for example relative to no administration of the Gsx1 protein Gsx1-CPP protein, or nucleic acid molecule encoding such), (5) increasing locomotion of the subject (e.g., as determined by Basso Mouse Scale (BMS) score or Functional Independence Measure (FIM)) (for example an increase of at least 5%, at least 10%, at least 15%, at least 20%, at least 50%, at least 75%, at least 90%, at least 100%, at least 200%, at least 300%, or at least 600%, for example relative to no administration of the Gsx1 protein, Gsx1-CPP protein, or nucleic acid molecule encoding such), (6) decreasing cell death, for example at or near the injury site, such as decreasing the number of cleaved caspase3 positive cells (such as a decrease of at least 5%, at least 10%, at least 15%, at least 20%, at least 50%, at least 75%, or at least 90% for example relative to no administration of the Gsx1 protein, Gsx1-CPP protein, or nucleic acid molecule encoding such), and (7) increasing neurogenesis, axon growth, and/or axon guidance, for example at or near the injury site, (e.g., as determined by Ctnna1 and/or Col6a2 expression, Netrin signaling, expression of axonal guidance genes, and/or CREB signaling) (such as an increase of at least 5%, at least 10%, at least 15%, at least 20%, at least 50%, at least 75%, at least 90%, at least 100%, at least 200%, at least 300%, or at least 500%, for example relative to no administration of the Gsx1 protein, Gsx1-CPP protein, or nucleic acid molecule encoding such). In some examples, such responses are achieved within about 3 days, within about 1 week, within about 2 weeks, within about 4 weeks, within about 8 weeks, within about 12 weeks, with in about 4 months, within about 6 months, or within about 52 weeks following treatment. In some embodiments, the disclosed methods include measuring inflammation, cell proliferation, astrogliosis, glial scaring, neurogenesis, NSPC activation, and/or cell death, for example at or near an injury site, before and/or after treating a subject. In some embodiments, the disclosed methods include measuring locomotion of the subject before and after treating a subject.

In some embodiments, the disclosed methods include measuring locomotion before and/or after treating a subject. For example, functional outcome after spinal cord injury in humans, can be determined or measured using the Modified Barthel Index (MBI), Functional Independence Measure (FIM), Quadriplegia Index of Function (QIF), and/or the Spinal Cord Independence Measure (SCIM). Examples of such methods are described in Furlan et al., Journal of Neurotrauma. 2011; 28(8):1413-1430; Chumney et al., (2010). The Journal of Rehabilitation Research and Development. 47 (1): 17-30; Ota et al., Spinal Cord. 1996; 34(9):531-5; and Functional Recovery Outcome Measures Work Group:, Anderson et al., The Journal of Spinal Cord Medicine. 2008; 31(2): 133-144.

In some examples, an amount of (1) inflammation, for example at or near the injury site, (2) proliferation of NSPCs, for example at or near the injury site, (3) differentiation of NSPCs towards a neuronal linage, such as glutamatergic neurons, for example at or near the injury site, (4) astrogliosis and glial scar formation, for example at or near the injury site, (5) locomotion of the subject, and/or (6) cell death, for example at or near the injury site, is compared to a control. In some embodiments, the control is a value obtained prior to treatment. In some embodiments, the control is a historical control or standard reference value or range of values (such as a previously tested control sample, such as a group of subjects with or without a neurological disorder). In further examples, the control is a reference value, such as a standard value obtained from a population of normal individuals, or individual known to have a neurological disorder (such as a SCI or TBI). Similar to a control population, the value the value obtained from the treated subject can be compared to the mean reference value or to a range of reference values (such as the high and low values in the reference group or the 95% confidence interval). In other examples, the control is the subject (or group of subjects) treated with placebo compared to the same subject (or group of subjects) treated with the Gsx1 protein, Gsx1-CPP protein, or nucleic acid molecule encoding such in a cross-over study. In further examples, the control is the subject (or group of subjects) prior to treatment with the Gsx1 protein, Gsx1-CPP protein, or nucleic acid molecule encoding such.

A. Gsx1 Proteins

Exemplary full-length Gsx1 proteins are shown in SEQ ID NOS: 2 (human) and 4 (mouse). In some examples, a Gsx1 protein includes or consists of the protein sequence of SEQ ID NO: 2 or 4. In some examples, a Gsx1 protein includes or consists of the protein sequence comprising at least 80%, at least 85%, at least 90%, at least 95%, at least 98%, at least 99%, or 100% sequence identity to SEQ ID NO: 2 or 4. Exemplary Gsx1 coding sequences are shown in SEQ ID NOS: 1 (human) and 3 (mouse). In some examples, a Gsx1 nucleic acid sequence includes or consists of the sequence of SEQ ID NO: 1 or 3, which in some examples is part of a plasmid or vector, and in some examples operably linked to a promoter (such as a constitutive or CNS-specific promoter).

In one example, the disclosed methods utilize a Gsx1 protein (such as a mammalian Gsx1 protein), that is, a Gsx1 protein is administered to the subject. In some other examples, the disclosed methods utilize a Gsx1-CPP fusion protein (such as a fusion protein composed of a mammalian Gsx1 protein and a cell penetrating peptide), that is, a Gsx1-CPP fusion protein is administered to the subject. Examples of Gsx1 proteins, which can be used to generate the Gsx1 domain of a Gsx1-CPP protein. are shown in SEQ ID NOS: 2 (human) and 4 (mouse). Native or variant Gsx1 proteins can be used. In one example, variant Gsx1 peptides are produced by manipulating a Gsx1 nucleotide sequence. In some examples a variant Gsx1 sequence is used, such as one including amino acid substitutions, additions, deletions, or combinations thereof, as long as the protein retains the ability to increase neurogenesis, reduce astrogliosis and glial scar formation, and increase locomotion following spinal cord injury. Methods of measuring neurogenesis, astrogliosis and glial scar formation, and locomotion are described herein. Regions of Gsx1 that are more likely to tolerate substitution can be determined by aligning sequences (e.g., SEQ ID NOS: 2 and 4), wherein amino acids conserved between species are less likely to tolerate substitutions, while amino acids that vary at a particular positon are more likely to tolerate substitutions.

Variant Gsx1 proteins, such as variants of SEQ ID NOS: 2 and 4, can contain one or more mutations, such as a single insertion, a single deletion, a single substitution. In some examples, a variant Gsx1 protein includes 1-20 insertions, 1-20 deletions, 1-20 substitutions, or any combination thereof (e.g., single insertion together with 1-19 substitutions). In some examples, the variant Gsx1 protein (e.g., SEQ ID NO: 2 or 4) has 1, 2, 3, 4, 5, 6, 7, 8, 9, 10, 11, 12, 13, 14, 15, 16, 17, 18, 19 or 20 amino acid changes. In some examples, SEQ ID NO: 2 or 4 has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid changes, such as 1-8 insertions, 1-15 deletions, 1-10 substitutions, or any combination thereof (e.g., 1-15, 1-4, or 1-5 amino acid deletions together with 1-10, 1-5 or 1-7 amino acid substitutions). One type of modification includes the substitution of amino acids for amino acid residues having a similar biochemical property, that is, a conservative substitution (such as 1-4, 1-8, 1-10, or 1-20 conservative substitutions). Typically, conservative substitutions have little to no impact on the activity of a resulting peptide. For example, a conservative substitution is an amino acid substitution in SEQ ID NO: 2 or 4 that does not substantially affect the ability of the Gsx1 peptide to increase neurogenesis, reduce astrogliosis and glial scar formation, and increase locomotion following spinal cord injury, in a mammal. An alanine scan can be used to identify which amino acid residues in a Gsx1 protein (or Gsx1-CPP protein), such as SEQ ID NO: 2 or 4, can tolerate an amino acid substitution. In one example, these activities of Gsx1, (e.g., SEQ ID NO: 2 or 4), are not altered by more than 25%, for example not more than 20%, for example not more than 10%, when an alanine, or other conservative amino acid, is substituted for 1-4, 1-8, 1-10, or 1-20 native amino acids. Examples of amino acids which may be substituted for an original amino acid in a protein and which are regarded as conservative substitutions include: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

More substantial changes can be made by using substitutions that are less conservative, e.g., selecting residues that differ more significantly in their effect on maintaining: (a) the structure of the polypeptide backbone in the area of the substitution, for example, as a sheet or helical conformation; (b) the charge or hydrophobicity of the polypeptide at the target site; or (c) the bulk of the side chain. The substitutions that in general are expected to produce the greatest changes in polypeptide function are those in which: (a) a hydrophilic residue, e.g., serine or threonine, is substituted for (or by) a hydrophobic residue, e.g., leucine, isoleucine, phenylalanine, valine or alanine; (b) a cysteine or proline is substituted for (or by) any other residue; (c) a residue having an electropositive side chain, e.g., lysine, arginine, or histidine, is substituted for (or by) an electronegative residue, e.g., glutamic acid or aspartic acid; or (d) a residue having a bulky side chain, e.g., phenylalanine, is substituted for (or by) one not having a side chain, e.g., glycine. The effects of these amino acid substitutions (or other deletions or additions) can be assessed by analyzing the function of the Gsx1 protein, such as SEQ ID NO: 2 or 4, or a or Gsx1-CPP protein, by analyzing the ability of the variant Gsx1 protein to increase neurogenesis, reduce astrogliosis and glial scar formation, and increase locomotion following spinal cord injury, in a mammal.

In some examples, a Gsx1 protein (or Gsx1-CPP protein) used in the disclosed methods is PEGylated at one or more positions. In some examples, a Gsx1 protein (or Gsx1-CPP protein) used in the disclosed methods includes an immunoglobin FC domain. The conserved FC fragment of an antibody can be incorporated either n-terminal or c-terminal of the Gsx1 protein, and can enhance stability of the protein and therefore serum half-life. The FC domain can also be used as a means to purify the proteins on protein A or Protein G sepharose beads.

Gsx1 proteins can be tagged with cell penetrating peptide (CPP) to promote cellular uptake. Thus, in some examples, the Gsx1 protein used is a fusion or chimeric protein, that includes (1) a Gsx1 protein, and (2) a cell penetrating peptide (referred to herein as a Gsx1-CPP fusion protein). The cell penetrating peptide domain can be at the N- or C-terminus of the Gsx1 protein domain. Cell penetrating peptides are usually short peptides (40 amino acids or less) that are highly cationic and usually rich in arginine and lysine that can facilitate cellular intake/uptake of proteins. Exemplary cell penetrating peptides that can be used include hydrophilic peptides (e.g., TAT [YGRKKRRQRRR; SEQ ID NO: 61], SynB1 [RGGRLSYSRRRFSTSTGR; SEQ ID NO: 62], SynB3 [RRLSYSRRRF ; SEQ ID NO: 63], PTD-4 [PIRRRKKLRRLK ; SEQ ID NO: 64], PTD-5 [RRQRRTSKLMKR; SEQ ID NO: 65], FHV Coat-(35-49) [RRRRNRTRRNRRRVR; SEQ ID NO: 66], BMV Gag-(7-25) [KMTRAQRRAAARRNRWTAR; SEQ ID NO: 67], HTLV-II Rex-(4-16) [TRRQRTRRARRNR; SEQ ID NO: 68], D-Tat [GRKKRRQRRRPPQ; SEQ ID NO: 69], R9-Tat [G PQ; SEQ ID NO: 70] and penetratin [RQIKWFQNRRMKWKK; SEQ ID NO: 71]), amphiphilic peptides (e.g., MAP [KLALKLALKLALALKLA; SEQ ID NO: 72], SBP [MGLGLHLLVLAAALQGAWSQPKKKRKV; SEQ ID NO: 73], FBP [GALFLGWLGAAGSTMGAWSQPKKKRKV; SEQ ID NO: 74], MPG ac-GALFLGFLGAAGSTMGAWSQPKKKRKV-cya; SEQ ID NO: 75], MPG(ΔNLS) [ac-GALFLGFLGAAGSTMGAWSQPKSKRKV-cya; SEQ ID NO: 76], Pep-2 [ac-KETWFETWFTEWSQPKKKRKV-cya; SEQ ID NO: 77], and transportan [GWTLNSAGYLLGKINLKALAALAKKIL; SEQ ID NO: 78]), periodic sequences (e.g., pVec, polyarginines RxN (4<N<17) chimera, polylysines KxN (4<N<17) chimera, (RAca)6R, (RAbu)6R, (RG)6R, (RM)6R, (RT)6R, (RS)6R, R10, (RA)6R, R7, and pep-1 [ac-KETWWETWWTEWSQPKKKRKV-cya; SEQ ID NO: 79]), and Cr10 (a cyclic pol-arginine CPP).

B. Generation of Gsx1 Proteins

Isolation and purification of recombinantly expressed Gsx1 proteins (and Gsx1-CPP proteins) can be carried out by conventional means, such as preparative chromatography and immunological separations. Once expressed, Gsx1 proteins (and Gsx1-CPP proteins) can be purified according to standard procedures, including ammonium sulfate precipitation, affinity columns, column chromatography, and the like (see, generally, R. Scopes, Protein Purification, Springer-Verlag, N.Y., 1982). Substantially pure compositions of at least about 90 to 95% homogeneity are disclosed herein, and 98 to 99% or more homogeneity can be used for pharmaceutical purposes.

In addition to recombinant methods, Gsx1 proteins (and Gsx1-CPP proteins) can also be constructed in whole or in part using standard peptide synthesis. In one example, Gsx1 proteins (and Gsx1-CPP proteins) are synthesized by condensation of the amino and carboxyl termini of shorter fragments. Peptide bonds can be formed by activation of a carboxyl terminal end (such as by the use of the coupling reagent N,N′-dicylohexylcarbodimide).

C. Gsx1 Nucleic Acid Molecules and Vectors

Exemplary Gsx1 coding sequences are shown in SEQ ID NOS: 1 (human) and 3 (mouse). In some examples, a Gsx1 encoding nucleic acid molecule includes or consists of the sequence of SEQ ID NO: 1 or 3. In some examples, a Gsx1 nucleic acid molecule encodes the protein of SEQ ID NO: 2 or 4, or a variant thereof (such as those described above). In some examples, a Gsx1 encoding nucleic acid sequence includes or consists of the sequence of SEQ ID NO: 1 or 3, which in some examples is part of a plasmid or vector, and in some examples operably linked to a promoter (such as a constitutive or CNS-specific promoter). In one example, the nucleic acid molecule encodes a Gsx1-CPP fusion protein, and can include a portion that encodes a Gsx1 portion (e.g., encodes the protein of SEQ ID NO: 2 or 4, or includes or consists of the sequence of SEQ ID NO: 1 or 3), and includes a portion that encodes a CPP (e.g., encodes any one of SEQ ID NOS: 61-79). In one example, a Gsx1-CPP fusion protein is encoded by two or more separate nucleic acid molecules, such as separate vectors, such as a first nucleic acid molecule that encodes a Gsx1 domain (e.g., encodes the protein of SEQ ID NO: 2 or 4, or includes or consists of the sequence of SEQ ID NO: 1 or 3), and a second nucleic acid molecule that encodes a CPP domain (e.g., encodes any one of SEQ ID NOS: 61-79).

In one example, the disclosed methods utilize a Gsx1 (or Gsx1-CPP) nucleic acid sequence (such as a cDNA, genomic, or RNA sequences), that is, a Gsx1 (or Gsx1-CPP) nucleic acid molecule is administered to the subject, and the Gsx1 (or Gsx1-CPP) protein encoded expressed in the cell where the nucleic acid molecule is introduced. The Gsx1 (or Gsx1-CPP) nucleic acid molecule can encode a native or variant Gsx1 protein as described above.

Based on the genetic code, nucleic acid sequences coding for any Gsx1 protein (e.g., SEQ ID NO: 2 or 4), or Gsx1-CPP can be generated. In some examples, such a sequence is optimized for expression in a host cell, such as a host cell used to express the Gsx1 (or Gsx1-CPP) protein. Such nucleic acids can be used directly (e.g., administered to a subject), or used to produce a Gsx1 (or Gsx1-CPP) protein which is administered to a subject.

In one example, the nucleic acid molecule encoding a Gsx1 protein (or Gsx1 domain of Gsx1-CPP) comprises or consists of the sequence of SEQ ID NO: 1 or 3. Also provided are cells, plasmids and viral vectors including such nucleic acids, which can also include a promoter operably linked to the Gsx1 (or Gsx1-CPP) coding sequence.

In one example, a nucleic acid sequence the encodes for a Gsx1 protein has at least 60%, at least 70%, at least 75%, at least 80%, at least 90%, at least 92%, at least 95%, at least 96%, at least 97%, at least 98%, at least 99% or 100% sequence identity to SEQ ID NO: 1 or 3. Thus, in some examples, the Gsx1 portion of a Gsx1-CPP fusion protein is encoded by a nucleic acid the having at least 60%, at least 70%, at least 75%, at least 80%, at least 90%, at least 92%, at least 95%, at least 96%, at least 97%, at least 98%, at least 99%, or 100% sequence identity to SEQ ID NO: 1 or 3. Such sequences can readily be produced, using the amino acid sequences provided herein and that are publicly available, and the genetic code. In addition, one of skill can readily construct a variety of clones containing functionally equivalent nucleic acids, such as nucleic acids which differ in sequence but which encode the same Gsx1 protein sequence.

Nucleic acid molecules include DNA, cDNA, mRNA, and RNA sequences which encode a Gsx1 protein. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3^{rd} Edition, W.H. 5 Freeman and Co., NY).

Codon preferences and codon usage tables for a particular species can be used to engineer isolated nucleic acid molecules encoding a Gsx1 protein (such as one encoding a protein having at least 90%, at least 95%, at least 96%, at least 97%, at least 98%, at least 99% or 100% sequence identity to SEQ ID NO: 2 or 4) that take advantage of the codon usage preferences of that particular species. For example, the Gsx1 proteins used in the disclosed methods can be designed to have codons that are preferentially used by a particular organism of interest (such as a human or mouse).

A nucleic acid encoding a Gsx1 protein (such as a nucleic acid molecule having at least 90%, at least 95%, at least 96%, at least 97%, at least 98%, at least 99% or 100% sequence identity to SEQ ID NO: 1 or 3, or encoding a protein having at least 90%, at least 95%, at least 96%, at least 97%, at least 98%, at least 99% or 100% sequence identity to SEQ ID NO: 2 or 4), or a Gsx1-CPP fusion protein, can be cloned or amplified by in vitro methods, such as the polymerase chain reaction (PCR), the ligase chain reaction (LCR), the transcription-based amplification system (TAS), the self-sustained sequence replication system (3 SR) and the Qβ replicase amplification system (QB). In addition, nucleic acids encoding a Gsx1 protein (such as a nucleic acid molecule having at least 90%, at least 95%, at least 96%, at least 97%, at least 98%, at least 99% or 100% sequence identity to SEQ ID NO: 1 or 3, or encoding a protein having at least 90%, at least 95%, at least 96%, at least 97%, at least 98%, at least 99% or 100% sequence identity to SEQ ID NO: 2 or 4), or a Gsx1-CPP fusion protein, can be prepared by cloning techniques (such as those found in Sambrook et al. (ed.), Molecular Cloning: A Laboratory Manual 2nd ed., vol. 1-3, Cold Spring Harbor Laboratory Press, Cold Spring, Harbor, N.Y., 1989, and Ausubel et al., (1987) in “Current Protocols in Molecular Biology,” John Wiley and Sons, New York, N.Y.).

Nucleic acid sequences encoding a Gsx1 protein (such as a nucleic acid molecule having at least 90%, at least 95%, at least 96%, at least 97%, at least 98%, at least 99% or 100% sequence identity to SEQ ID NO: 1 or 3, or encoding a protein having at least 90%, at least 95%, at least 96%, at least 97%, at least 98%, at least 99% or 100% sequence identity to SEQ ID NO: 2 or 4), or a Gsx1-CPP fusion protein, can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is generally limited to sequences of about 100 bases, longer sequences may be obtained by the ligation of shorter sequences.

In one example, a Gsx1 protein (such as on having at least 90%, at least 95%, at least 96%, at least 97%, at least 98%, at least 99% or 100% sequence identity to SEQ ID NO: 2 or 4) is prepared by inserting a cDNA which encodes a Gsx1 protein (such as a nucleic acid molecule having at least 90%, at least 95%, at least 96%, at least 97%, at least 98%, at least 99% or 100% sequence identity to SEQ ID NO: 1 or 3) into a vector. The insertion can be made so that the Gsx1 protein is read in frame so that the Gsx1 protein is produced. Similar methods can be used to encode a Gsx1-CPP fusion protein.

The Gsx1 nucleic acid coding sequence (such as a nucleic acid molecule having at least 90%, at least 95%, at least 96%, at least 97%, at least 98%, at least 99% or 100% sequence identity to SEQ ID NO: 1 or 3, or encoding a protein having at least 90%, at least 95%, at least 96%, at least 97%, at least 98%, at least 99% or 100% sequence identity to SEQ ID NO: 2 or 4) can be inserted into an expression vector including, but not limited to a plasmid, virus or other vehicle that can be manipulated to allow insertion or incorporation of sequences and can be expressed in either prokaryotes or eukaryotes. Hosts can include microbial, yeast, insect, plant and mammalian organisms. The vector can encode a selectable marker, such as a thymidine kinase gene, antibiotic resistance gene, or fluorescent protein. Similar methods can be used for a nucleic acid encoding a Gsx1-CPP fusion protein.

Nucleic acid sequences encoding a Gsx1 protein (such as a nucleic acid molecule having at least 90%,at least 95%, at least 96%, at least 97%, at least 98%, at least 99% or 100% sequence identity to SEQ ID NO: 1 or 3, or encoding a protein having at least 90%, at least 95%, at least 96%, at least 97%, at least 98%, at least 99% or 100% sequence identity to SEQ ID NO: 2 or 4) can be operatively linked to expression control sequences. An expression control sequence operatively linked to a Gsx1 protein coding sequence is ligated such that expression of the Gsx1 coding sequence is achieved under conditions compatible with the expression control sequences. Exemplary expression control sequences include, but are not limited to promoters, enhancers, transcription terminators, a start codon (i.e., ATG) in front of a Gsx1 protein-encoding gene, splicing signal for introns, maintenance of the correct reading frame of that gene to permit proper translation of mRNA, and stop codons. Similar methods can be used for a nucleic acid encoding a Gsx1-CPP fusion protein.

In one embodiment, vectors are used for expression in yeast such as S. cerevisiae, P. pastoris, or Kluyveromyces lactis. Exemplary promoters for use in yeast expression systems include the constitutive promoters, plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, inducible promoters are of use, such as GAL1-10 (induced by galactose), PHOS (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2 μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The nucleic acid molecules encoding a Gsx1 protein (such as one having at least 90%, at least 95%, at least 96%, at least 97%, at least 98%, at least 99% or 100% sequence identity to SEQ ID NO: 1 or 3, or encoding a protein having at least 90%, at least 95%, at least 96%, at least 97%, at least 98%, at least 99% or 100% sequence identity to SEQ ID NO: 2 or 4), or a Gsx1-CPP encoding nucleic acid molecule, can also be designed to express in insect cells.

A Gsx1 protein (such as one having at least 90%, at least 95%, t least 96%, at least 97%, at least 98%, at least 99% or 100% sequence identity to SEQ ID NO: 2 or 4), or a Gsx1-CPP, can be expressed in a yeast strain. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast cell lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared that encode a Gsx1 protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecule that encodes a protein having at least 90%, at least 95%, at least 96%, at least 97%, at least 98%, at least 99% or 100% sequence identity to SEQ ID NO: 2 or 4), or encode a Gsx1-CPP. Exemplary viral vectors include polyoma, SV40, adenovirus, vaccinia virus, adeno-associated virus (AAV), herpes viruses including HSV and EBV, Sindbis viruses, alphaviruses and retroviruses of avian, murine, and human origin. Baculovirus (Autographa californica multinuclear polyhedrosis virus; AcMNPV) vectors can also be used. Other suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

Viral vectors that encode a Gsx1 protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecules that encodes a protein having at least 90%, at least 95%, at least 96%, at least 97%, at least 98%, at least 99% or 100% sequence identity to SEQ ID NO: 2 or 4) can include at least one expression control element operationally linked to the nucleic acid sequence encoding the Gsx1 protein. The expression control elements are inserted in the vector to control and regulate the expression of the nucleic acid sequence. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding the Gsx1 protein in the host system. The expression vector can contain additional elements necessary for the transfer and subsequent replication of the expression vector containing the nucleic acid sequence in the host system. Examples of such elements include, but are not limited to, origins of replication and selectable markers. Such vectors can be constructed using conventional methods (Ausubel et al., (1987) in “Current Protocols in Molecular Biology,” John Wiley and Sons, New York, N.Y.) and are commercially available. Similar vectors can be used with a nucleic acid encoding a Gsx1-CPP fusion protein.

In one example, the viral vector that encodes a Gsx1 protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecule that encodes a protein having at least 90%, at least 95%, at least 96%, at least 97%, at least 98%, at least 99% or 100% sequence identity to SEQ ID NO: 2 or 4) is a lentiviral or AAV vector, and includes a promoter operably linked to the Gsx1 coding sequence. In some examples, the promoter is a constitutive promoter, such as CMV, beta actin, or T7, or a tissue specific promoter, such as a s CNS-specific promoter (e.g., synapasin 1 (Syn1) promoter, glial fibrillary acidic protein (GFAP) promoter, nestin (NES) promoter, myelin-associated oligodendrocyte basic protein (MOBP) promoter, myelin basic protein (MBP) promoter, tyrosine hydroxylase (TH) promoter, or a forkhead box A2 (FOXA2) promoter). Similar promoters can be used with a nucleic acid encoding a Gsx1-CPP fusion protein.

Methods for preparing recombinant virus containing a heterologous DNA sequence encoding the Gsx1 protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecules that encodes a protein having at least 90%, at least 95%, at least 96%, at least 97%, at least 98%, at least 99% or 100% sequence identity to SEQ ID NO: 2 or 4) are known. Such techniques involve, for example, homologous recombination between the viral sequences flanking the Gsx1 coding sequence in a donor plasmid and homologous sequences present in the parental virus. The vector can be constructed for example by using a unique restriction endonuclease site that is naturally present or artificially inserted in the parental viral vector to insert the heterologous DNA. Similar methods can be used for a nucleic acid encoding a Gsx1-CPP fusion protein.

When the cell into which the Gsx1 coding sequence is introduced is eukaryotic, transfection methods include calcium phosphate coprecipitates, mechanical procedures such as microinjection, electroporation, insertion of a plasmid encased in liposomes, or viral vectors. Eukaryotic cells can also be co-transformed with polynucleotide sequences encoding a Gsx1 protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecule that encodes a protein having at least 90%, at least 95%, at least 96%, at least 97%, at least 98%, at least 99% or 100% sequence identity to SEQ ID NO: 2 or 4), and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Expression systems such as plasmids and vectors can be used to produce Gsx1 proteins in cells including higher eukaryotic cells such as the COS, CHO, HeLa and myeloma cell lines. Similar cells can be used with a nucleic acid encoding a Gsx1-CPP fusion protein.

D. Cells Expressing Gsx1 Protein

A nucleic acid molecule encoding a Gsx1 protein or a Gsx1-CPP fusion protein can be used to transform cells and make transformed cells. Thus, cells expressing a Gsx1 protein or a Gsx1-CPP fusion protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecule that encodes a protein (or Gsx1 portion thereof) having at least 90%, at least 95%, at least 96%, at least 97%, at least 98%, at least 99% or 100% sequence identity to SEQ ID NO: 2 or 4), are disclosed. In one example, the cell is a mammalian cell present in a mammal, for example to treat a neurological disorder. In another example, the cell is in culture (e.g., ex vivo or in vitro), for example used to express a therapeutic Gsx1 protein or a Gsx1-CPP fusion protein, such as a eukaryotic or prokaryotic cell (e.g., bacteria, archea, plant, fungal, yeast, insect, and mammalian cells, such as Lactobacillus, Lactococcus, Bacillus (such as B. subtilis), Escherichia (such as E. coli), Clostridium, Saccharomyces or Pichia (such as S. cerevisiae or P. pastoris), Kluyveromyces lactis, Salmonella typhimurium, SF9 cells, C129 cells, 293 cells, Neurospora, and immortalized mammalian neurological cell lines).

Cells expressing a Gsx1 protein or a Gsx1-CPP fusion protein are transformed or recombinant cells. Such cells can include at least one exogenous nucleic acid molecule that encodes a Gsx1 protein or a Gsx1-CPP fusion protein (such as those that include a nucleic acid molecule having at least 90%, at least 95%, at least 96%, at least 97%, at least 98%, at least 99% or 100% sequence identity to SEQ ID NO: 1 or 3, or a nucleic acid molecules that encodes a protein (or Gsx1 portion thereof) having at least 90%, at least 95%, at least 96%, at least 97%, at least 98%, at least 99% or 100% sequence identity to SEQ ID NO: 2 or 4). It is understood that all progeny may not be identical to the parental cell since there may be mutations that occur during replication. In one example, the one exogenous nucleic acid molecule that encodes a Gsx1 protein is stably transferred, meaning that the foreign DNA is continuously maintained in the transformed cell.

Transformation of a host cell with recombinant nucleic acid may be carried out by conventional techniques. Where the host is prokaryotic, such as E. coli, competent cells which are capable of DNA uptake can be prepared from cells harvested after exponential growth phase and subsequently treated with CaCl₂, MgCl₂ or RbCl. Transformation can also be performed after forming a protoplast of the host cell if desired, using polyethylene glycol transformation, or by electroporation. Examples of commonly used mammalian host cell lines are HEK293T cells, VERO and HeLa cells, CHO cells, and WI38, BHK, and COS cell lines.

E. Pharmaceutical Compositions and Dosing

Pharmaceutical compositions that include a Gsx1 protein or a Gsx1-CPP fusion protein, or nucleic acid molecule encoding such, are provided. In one example, a composition includes an isolated Gsx1 protein having at least 80%, at least 85%, at least 90%, at least 95%, at least 98%, at least 99%, or 100% sequence identity to SEQ ID NO: 2 or 4, for example encapsulated in a liposome. In one example, a composition includes an isolated Gsx1-cell penetrating peptide (CPP) fusion protein comprising a Gxx1 portion comprising at least 80%, at least 85%, at least 90%, at least 95%, at least 98%, at least 99%, or 100% sequence identity to SEQ ID NO: 2 or 4, and a CPP portion, wherein the Gsx1 portion and the CPP portion are optionally joined by a linker. In some examples, the CPP portion has at least 80%, at least 85%, at least 90%, at least 95%, at least 98%, at least 99%, or 100% sequence identity to any one of SEQ ID NOS: 61-79. In some examples, the Gsx1-CPP fusion protein in a composition is encapsulated in a liposome. In one example, a composition includes an isolated nucleic acid molecule encoding a Gsx1 protein having at least 80%, at least 85%, at least 90%, at least 95%, at least 98%, at least 99%, or 100% sequence identity to SEQ ID NO: 1 or 3 or encoding a Gsx1 protein having at least 80%, at least 85%, at least 90%, at least 95%, at least 98%, at least 99%, or 100% sequence identity to SEQ ID NO: 2 or 4. In one example, a composition includes an isolated nucleic acid molecule encoding a Gsx1-CPP fusion protein, wherein a Gsx1 portion of the Gsx1-CPP fusion protein is encoded by a nucleic acid molecule having at least 80%, at least 85%, at least 90%, at least 95%, at least 98%, at least 99%, or 100% sequence identity to SEQ ID NO: 1 or 3 or encodes a Gsx1 protein having at least 80%, at least 85%, at least 90%, at least 95%, at least 98%, at least 99%, or 100% sequence identity to SEQ ID NO: 2 or 4, and wherein optionally a nucleic acid molecule encoding a CPP portion of the Gsx1-CPP fusion protein encodes a protein comprising at least 80%, at least 85%, at least 90%, at least 95%, at least 98%, at least 99%, or 100% sequence identity to any one of SEQ ID NOS: 61-79. In some examples, the nucleic acid molecule in a composition is encapsulated in a liposome. Such compositions can also include a pharmaceutically acceptable carrier.

In some embodiments, the pharmaceutical composition consists essentially of a Gsx1 protein (such as a protein having at least 90%, at least 95%, at least 96%, at least 97%, at least 98%, at least 99% or 100% sequence identity to SEQ ID NO: 2 or 4), a Gsx1 fusion protein composed of (1) a Gsx1 protein comprising at least 80%, at least 85%, at least 90%, at least 95%, at least 98%, at least 99%, or 100% sequence identity to SEQ ID NO: 2 or 4 and (2) a cell penetrating peptide (such as a protein comprising at least 80%, at least 85%, at least 90%, at least 95%, at least 98%, at least 99%, or 100% sequence identity to any one of SEQ ID NOS: 61-79), or a nucleic acid encoding a Gsx1 protein (such as a nucleic acid molecule having at least 90%, at least 95%, at least 96%, at least 97%, at least 98%, at least 99% or 100% sequence identity to SEQ ID NO: 1 or 3, or a nucleic acid molecule that encodes a Gsx1 protein or a Gsx1-CPP fusion protein, optionally the Gsx1 protein or a Gsx1-CPP fusion protein (or nucleic acid molecule encoding such) is encapsulated in a liposome, and a pharmaceutically acceptable carrier. In these embodiments, additional therapeutically effective agents are not included in the compositions.

Such compositions can be formulated with an appropriate pharmaceutically acceptable carrier (such as water or saline), depending upon the particular mode of administration chosen. Such compositions can be administered to a subject with a neurological disorder using the disclosed methods. In one example, the pharmaceutical composition is suitable for injection, such as injection into the CNS, for example at or near the site of injury (e.g., rostral and/or caudal to the injury site). In some examples, intraparenchymal, introcerebroventricular, or intrathecal (cisternal and lumbar) injections are used to target brain and/or spinal cord.

The pharmaceutical composition can include a therapeutically effective amount of another agent. Examples of such agents include, without limitation, those listed in section “F” below, or combinations thereof.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

In some embodiments, the Gsx1 protein, Gsx1-CPP fusion protein, or nucleic acid molecule encoding such is included in a controlled release formulation, for example, a microencapsulated formulation. Various types of biodegradable and biocompatible polymers, methods can be used, and methods of encapsulating a variety of synthetic compounds, proteins and nucleic acids can be used (see, for example, U.S. Patent Publication Nos. 2007/0148074; 2007/0092575; and 2006/0246139; U.S. Pat. Nos. 4,522, 811; 5,753,234; and 7,081,489; PCT Publication No. WO/2006/052285; Benita, Microencapsulation: Methods and Industrial Applications, 2^{nd} ed., CRC Press, 2006).

In other embodiments, the Gsx1 protein, Gsx1-CPP fusion protein, or nucleic acid molecule encoding such, is included in a nanodispersion system. See, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers that can be used include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006. With regard to the administration of nucleic acids, one approach to administration of nucleic acids is direct treatment with a viral vector, such as a lentiviral or AAV vector. As described above, the nucleotide sequence encoding a Gsx1 protein (such as a protein having at least 90%, at least 95%, at least 96%, at least 97%, at least 98%, at least 99% or 100% sequence identity to SEQ ID NO: 2 or 4, or such as a nucleic acid molecule having at least 90%, at least 95%, at least 96%, at least 97%, at least 98%, at least 99% or 100% sequence identity to SEQ ID NO: 1 or 3, or a nucleic acid molecule that encodes a protein having at least 90%, at least 95%, at least 96%, at least 97%, at least 98%, at least 99% or 100% sequence identity to SEQ ID NO: 2 or 4) can be placed under the control of a promoter to increase expression of the Gsx1 protein.

Many types of release delivery systems can be used. Examples include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the Gsx1 protein, Gsx1-CPP fusion protein, or nucleic acid molecule encoding such, is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

A long-term sustained release implant can be suitable for treatment of chronic conditions, such as neurological disorders. Long-term release, as used herein, means that the implant is constructed and arranged to deliver therapeutic levels of the active ingredient for at least 30 days, or at least 60 days. Long-term sustained release implants include some of the release systems described above. These systems have been described for use with nucleic acids (see U.S. Pat. No. 6,218,371). For use in vivo, nucleic acids and peptides are relatively resistant to degradation (such as via endo- and exo-nucleases). Thus, modifications of a Gsx1 protein, such as the inclusion of a C-terminal amide, can be used.

The dosage form of the pharmaceutical composition can be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. For solid compositions, conventional non-toxic solid carriers can include pharmaceutical grades of mannitol, lactose, cellulose, starch, or magnesium stearate.

In some examples, the therapeutically effective amount of the Gsx1 protein, Gsx1-CPP fusion protein, or nucleic acid molecule encoding, such is the amount necessary to (1) decrease inflammation, for example at or near the injury site, such as decrease the number of infiltrated macrophages (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such), (2) increase the number of neural stem/progenitor cells (NSPCs) (e.g., as determined by measuring expression of nestin, and/or doublecortin), for example at or near the injury site, (for example an increase of at least 5%, at least 10%, at least 15%, at least 20%, at least 50%, at least 75%, at least 90%, or at least 100%, for example relative to no administration of the Gsx1 protein, Gsx1-CPP fusion protein, or nucleic acid molecule encoding such), (3) increase differentiation of NSPCs towards a specific neuronal linage, such as an increase in the number of glutamatergic neurons and cholinergic neurons (and decrease in the number of GABAergic interneurons) for example at or near the injury site (e.g., as determined by measuring expression of NeuN, ChAT, and/or Glut2) (for example an increase of at least 5%, at least 10%, at least 15%, at least 20%, at least 50%, at least 75%, at least 90%, at least 100%, at least 200%, at least 300%, or at least 600%, for example relative to no administration of the Gsx1 protein, Gsx1-CPP fusion protein, or nucleic acid molecule encoding such), decrease the number of GABAergic interneurons (e.g., as determined by measuring expression of GABA) (for example a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such) (4) decrease astrogliosis and glial scar formation, for example at or near the injury site, such as decreasing the number of astrocytes (e.g., as determined by measuring expression of GFAP, Serpina3n, and/or CSPG) (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such), (5) increase locomotion of the subject (e.g., as determined by Basso Mouse Scale (BMS) score or Functional Independence Measure (FIM)) (for example an increase of at least 5%, at least 10%, at least 15%, at least 20%, at least 50%, at least 75%, at least 90%, or at least 100%, at least 200%, at least 300%, or at least 600%, for example relative to no administration of the Gsx1 protein, Gsx1-CPP fusion protein, or nucleic acid molecule encoding such), and/or (6) decrease cell death, for example at or near the injury site, such as decrease the number of cleaved caspase3 positive cells (such as a decrease of at least 5%, at least 10%, at least 15%, at least 20%, at least 50%, at least 75%, or at least 90% for example relative to no administration of the Gsx1 protein, Gsx1-CPP fusion protein, or nucleic acid molecule encoding such).

The pharmaceutical compositions that include the Gsx1 protein, Gsx1-CPP fusion protein, or nucleic acid molecule encoding such, can be formulated in unit dosage form, suitable for individual administration of precise dosages. In one non-limiting example, a unit dosage contains from about 1 mg to about 1 g of a Gsx1 or Gsx1-CPP protein, such as about 10 mg to about 100 mg, about 50 mg to about 500 mg, about 100 mg to about 900 mg, about 250 mg to about 750 mg, or about 400 mg to about 600 mg. In other examples, a therapeutically effective amount of a Gsx1 or Gsx1-CPP protein is about 0.01 mg/kg to about 50 mg/kg, for example, about 0.5 mg/kg to about 25 mg/kg or about 1 mg/kg to about 10 mg/kg. In other examples, a therapeutically effective amount of a Gsx1 or Gsx1-CPP fusion protein is about 1 mg/kg to about 5 mg/kg, for example about 2 mg/kg. In a particular example, a therapeutically effective amount of a Gsx1 or Gsx1-CPP fusion protein is about 1 mg/kg to about 10 mg/kg, such as about 2 mg/kg.

Other suitable ranges include doses of Gsx1 protein or a Gsx1-CPP fusion protein of about 100 μg/kg to 10 mg/kg body weight or more (such as about 0.1-10 mg/kg, about 1-20 mg/kg, about 5-50 mg/kg, or about 10-100 mg/kg). In certain embodiments, the effective dosage is within narrower ranges of, for example, 5-40 mg/kg, 10-35 mg/kg or 20-25 mg/kg. In other examples, the dosage is about 1-100 mg, such as about 1-10 mg, about 5-25 mg, about 10-50 mg, about 25-60 mg, or about 50-100 mg (for example, about 1 mg, 5, mg, 10 mg, 15 mg, 20 mg, 25 mg, 30 mg, 35 mg, 40 mg, 45 mg, 50 mg, 55 mg, 60 mg, 70 mg, 80 mg, 90 mg, or 100 mg). In particular examples, the dose is about 20-60 mg, and in one non-limiting example, about 25 mg.

The pharmaceutical compositions that include a Gsx1 or Gsx1-CPP coding sequence can be formulated in unit dosage form, suitable for individual administration of precise dosages. Generally, the quantity of recombinant viral vector, carrying the nucleic acid coding sequence of Gsx1 protein or Gsx1-CPP fusion protein to be administered, is based on the titer of virus particles. In one non-limiting example, for example when a viral vector is utilized for administration of a nucleic acid encoding a Gsx1 protein or a Gsx1-CPP fusion protein, a unit dosage (e.g., 0.5-1.5 μl) contains about 10⁵ to about 10¹⁰ plaque forming units (pfu)/ml per mammal. Thus, in some examples, the recipient subject is administered a dose of about 10⁵ to about 10¹⁰ pfu/ml per mammal of recombinant virus in the composition. In some examples, the recipient subject is administered a dose of at least 10⁵ pfu/ml per mammal, at least 10⁶ pfu/ml per mammal at least 10⁷ pfu/ml per mammal at least 10⁸ pfu/ml per mammal, at least 10⁹ pfu/ml per mammal, or at least 10¹⁰ pfu/ml per mammal Examples of methods for administering the composition into mammals include, but are not limited to, injection of the composition into the affected tissue (such as into the brain or spinal cord) or intravenous, subcutaneous, intradermal or intramuscular administration of the virus.

The compositions of this disclosure that include a Gsx1 or Gsx1-CPP protein can be administered to humans or other animals by any means, including orally, intravenously, intramuscularly, intraperitoneally, intranasally, intradermally, intraparenchymally, introcerebroventricularly, intrathecally (e.g., cisternal and lumbar), subcutaneously, via inhalation or via suppository. In one non-limiting example, the composition is administered via injection. In some examples, site-specific administration of the composition can be used, for example by administering the Gsx1 protein, a Gsx1-CPP fusion protein, or coding sequence to CNS tissue (for example the brain or spinal cord, for example at or near the area of injury, such as rostral and/or caudal to the injury site). In some examples, administration is an intrathecal injection (e.g., of Gsx1 protein, Gsx1-CPP fusion protein, or nucleic acid molecule encoding such) to treat SCI in lumbar/sacral region, a cisterna magna injection (e.g., of Gsx1 protein, Gsx1-CPP fusion protein, or nucleic acid molecule encoding such) to treat SCI in cervical/thoracic region, or intraparenchymal or introcerebroventricular injection (e.g., of Gsx1 protein, Gsx1-CPP fusion protein, or nucleic acid molecule encoding such) to treat traumatic brain injury.

Treatment can involve a single administration, or multiple administrations (such as at least two separate administrations), such as doses over a period of a few days to months, or even years. For example, a therapeutically effective amount of Gsx1 protein, Gsx1-CPP fusion protein, or nucleic acid molecule encoding such can be administered in a single dose, or in several doses, for example daily, weekly, monthly, or yearly, during a course of treatment. In a particular non-limiting example, treatment involves administration once monthly, once yearly, or every-other-month. In some examples, where multiple doses are administered, the at least two separate administrations can be separated by at least 1 week, at least 2 weeks, at least 3 weeks, at least 4 weeks, at least 1 month, at least 2 months, at least 3 months, at least 6 months, at least 9 months, or at least one year.

In some examples, the first dose (and in some examples only dose) administrated occurs within 1 hour, within 2 hours, within 3 hours, within 4 hours, within 5 hours, within 6 hours, within 12 hours, within 24 hours, within 48 hours, within 72 hours, within 96 hours, within 1 week, within 2 weeks, within 3 weeks, within 4 weeks, within 1 month, within 2 months, or within 3 months of the onset of the neurological disorder, such as within 1 to 24 hours, 2 to 24 hours, 4 to 24 hours, or 1 to 96 hours of the onset of the neurological disorder.

F. Administration of Additional Therapy

In some examples, the Gsx1 protein, Gsx1-CPP fusion protein, or nucleic acid molecule encoding such, is administered in combination (such as sequentially, simultaneously, or contemporaneously) with one or more other agents, such as those useful in the treatment of a neurological disorder. The term “administration in combination” or “co-administration” refers to both concurrent and sequential administration of the active agents.

In some examples, a Gsx1 protein, a Gsx1-CPP fusion protein, or sequence encoding such is administered to a subject with a traumatic spinal cord or brain injury in combination with effective doses of one or more of stem cells, steroids (e.g., methylprednisolone), and iv fluids. In some examples, the subject also receives surgery, hypothermia treatment, or both. Administration of a Gsx1 protein or Gsx1 coding sequence, may also be in combination with lifestyle modifications, such as increased physical activity, physical therapy, or immobilization (e.g., in a hard collar).

In some examples, a Gsx1 protein or Gsx1 coding sequence is administered to a subject with a neurological disorder of the brain, such as Parkinson's disease, Alzheimer's disease, stroke (ischemic or hemorrhagic), ischemia, epilepsy, Huntington's disease, multiple sclerosis, amyotrophic lateral sclerosis, in combination with effective doses of one or more other therapeutic agents. For example, if the subject has Parkinson's disease, the method can further include administering a therapeutically effective amount of one or more of stem cells, deep brain stimulation, surgery (e.g., pallidotomy or thalamotomy) benztropine mesylate (Cogentin), entacapone (Comtan), dopar, dopamine agonist (e.g., apomorphine (Apokyn), pramipexole (Mirapex), ropinirole HCl (Requip), and rotigotine (Neupro)), larodopa, levodopa and carbidopa (Sinemet), rasagiline (Azilect), safinamide (Xadago), tasmar and trihexphenidyl (Artane). For example, if the subject has Alzheimer's disease, the method can further include administering a therapeutically effective amount of one or more of stem cells, a cholinesterase inhibitor (e.g., Razadyne® (galantamine), Exelon® (rivastigmine), or Aricept® (donepezil)), an N-methyl D-aspartate (NMDA) antagonist (e.g., memantine), Celexa® (citalopram), Remeron® (mirtazapine), Zoloft® (sertraline), Wellbutrin® (bupropion), Cymbalta® (duloxetine), and Tofranil® (imipramine). For example, if the subject has had a stroke, the method can further include administering a therapeutically effective amount of one or more of a tissue plasminogen activator (e.g., Alteplase IV r-tPA) for an ischemic stroke, or surgery (e.g., install a coil or clip to stop blood loss) for a hemorrhagic stroke. For example, if the subject has ischemia (e.g., cardiac ischemia or mesenteric artery ischemia), the method can further include administering a therapeutically effective amount of one or more of a vasodilator, anticoagulant, (e.g., heparin, aspirin), nitrate, ACE inhibitor, ranolazine, and surgery. For example, if the subject has epilepsy, the method can further include administering a therapeutically effective amount of one or more of an anti-seizure or anti-epileptic medication (e.g., carbamazepine, valproate, lamotrigine, dilantin or phenytek, ohenobarbital, tegretol or Carbatrol, mysoline, zarontin, depakene, depakote, depakote ER, valium and similar tranquilizers such as Tranxene and Klonopin, felbatol, gabitril, keppra, lamictal, lyrica, neurontin, topamax, trileptal, and, zonegran), surgery, vagus nerve stimulation, deep brain stimulation, and a ketogenic diet. For example, if the subject has Huntington's disease, the method can further include administering a therapeutically effective amount of one or more of a monoamine depletor (e.g., tetrabenazine or amantadine), SSRI antidepressant (e.g., fluoxetine citalopram, paroxetine, and sertraline) or other anti-depressant (e.g., amitriptyline, mirtazapine, duloxetine, and venlafaxine), antipsychotic drug (e.g., quetiapine, risperidone or olanzapine), mood-stabilizing drug (e.g., valproate or carbamazepine), and a high protein diet. For example, if the subject has multiple sclerosis, the method can further include administering a therapeutically effective amount of one or more of stem cells, a corticosteroid (e.g., methylprednisolone or prednisone), an interferon beta blocker (e.g., copaxone, teriflunomide, or mitoxantrone).

For example, if the subject has amyotrophic lateral sclerosis (ALS), the method can further include administering a therapeutically effective amount of one or more of a glutamate antagonist (e.g., riluzole) and a neuroprotective agent (e.g., edaravone). Thus, in some examples, the pharmaceutical composition that includes a Gsx1 protein, a Gsx1-CPP fusion protein, or sequence encoding such, further includes one or more of these therapeutic agents. Administration of a Gsx1 protein, a Gsx1-CPP fusion protein, or sequence encoding such, may also be in combination with increased physical activity, speech or language therapy, occupational therapy, physical therapy, or combinations thereof.

### IV. Compositions

Also provided are compositions, which can be used with the disclosed methods. In one example, the composition includes an isolated Gsx1 protein comprising at least 80%, at least 85%, at least 90%, at least 95%, at least 98%, at least 99%, or 100% sequence identity to SEQ ID NO: 2 or 4 and a liposome, wherein the Gsx1 protein is encapsulated in the liposome.

In one example, the composition includes a Gsx1 fusion protein composed of (1) a Gsx1 protein comprising at least 80%, at least 85%, at least 90%, at least 95%, at least 98%, at least 99%, or 100% sequence identity to SEQ ID NO: 2 or 4 and (2) a cell penetrating peptide (or a nucleic acid molecule encoding such a Gsx1-CPP fusion protein). Exemplary cell penetrating peptides that can be used include hydrophilic peptides (e.g., TAT [YGRKKRRQRRR; SEQ ID NO: 61], SynB1 [RGGRLSYSRRRFSTSTGR; SEQ ID NO: 62], SynB3 [RRLSYSRRRF; SEQ ID NO: 63], PTD-4 [PIRRRKKLRRLK; SEQ ID NO: 64], PTD-5 [RRQRRTSKLMKR; SEQ ID NO: 65], FHV Coat-(35-49) [RRRRNRTRRNRRRVR; SEQ ID NO: 66], BMV Gag-(7-25) [KMTRAQRRAAARRNRWTAR; SEQ ID NO: 67], HTLV-II Rex-(4-16) [TRRQRTRRARRNR; SEQ ID NO: 68], D-Tat [GRKKRRQRRRPPQ; SEQ ID NO: 69], R9-Tat [G PQ; SEQ ID NO: 70] and penetratin [RQIKWFQNRRMKWKK; SEQ ID NO: 71]), amphiphilic peptides (e.g., MAP [KLALKLALKLALALKLA; SEQ ID NO: 72], SBP [MGLGLHLLVLAAALQGAWSQPKKKRKV; SEQ ID NO: 73], FBP [GALFLGWLGAAGSTMGAWSQPKKKRKV; SEQ ID NO: 74], MPG ac-GALFLGFLGAAGSTMGAWSQPKKKRKV-cya; SEQ ID NO: 75], MPG(ANLS) [ac-GALFLGFLGAAGSTMGAWSQPKSKRKV-cya; SEQ ID NO: 76], Pep-2 [ac-KETWFETWFTEWSQPKKKRKV-cya; SEQ ID NO: 77], and transportan [GWTLNSAGYLLGKINLKALAALAKKIL; SEQ ID NO: 78]), periodic sequences (e.g., pVec, polyarginines RxN (4<N<17) chimera, polylysines KxN (4<N<17) chimera, (RAca)6R, (RAbu)6R, (RG)6R, (RM)6R, (RT)6R, (RS)6R, R10, (RA)6R, R7, and pep-1 [ac-KETWWETWWTEWSQPKKKRKV-cya; SEQ ID NO: 79]), and Cr10 (a cyclic pol-arginine CPP). Such a Gsx1 protein or Gsx1-CPP fusion protein can be encapsulated in a liposome.

In one example, a composition includes an isolated nucleic acid molecule encoding a Gsx1 protein having at least 80%, at least 85%, at least 90%, at least 95%, at least 98%, at least 99%, or 100% sequence identity to SEQ ID NO: 1 or 3 or encoding a Gsx1 protein having at least 80%, at least 85%, at least 90%, at least 95%, at least 98%, at least 99%, or 100% sequence identity to SEQ ID NO: 2 or 4. In one example, a composition includes an isolated nucleic acid molecule encoding a Gsx1-CPP fusion protein, wherein a Gsx1 portion of the Gsx1-CPP fusion protein is encoded by a nucleic acid molecule having at least 80%, at least 85%, at least 90%, at least 95%, at least 98%, at least 99%, or 100% sequence identity to SEQ ID NO: 1 or 3 or encodes a Gsx1 protein having at least 80%, at least 85%, at least 90%, at least 95%, at least 98%, at least 99%, or 100% sequence identity to SEQ ID NO: 2 or 4, and wherein optionally a nucleic acid molecule encoding a CPP portion of the Gsx1-CPP fusion protein encodes a protein comprising at least 80%, at least 85%, at least 90%, at least 95%, at least 98%, at least 99%, or 100% sequence identity to any one of SEQ ID NOS: 61-79. In some examples, the nucleic acid molecule in a composition is encapsulated in a liposome. Such compositions can also include a pharmaceutically acceptable carrier.

The disclosed compositions can further include other materials, such as a pharmaceutically acceptable carrier, such as water or saline, and/or other therapeutic agents, as described above.

### V. Programming Cells

Also provided are methods of in vitro or ex vivo programming of cells into specific neuronal cells (e.g., glutamatergic or cholinergic neurons). For example, a host cell, such as neural stem/progenitor cells (NSPCs), fibroblast cells, or embryonic stem cells (ESCs), can be transfected with a nucleic acid molecule provided herein (such as a viral vector or plasmid encoding a Gsx1 protein or Gsx1-CPP fusion protein). The Gsx1 nucleic acid molecule can encode a Gsx1 protein comprising at least 80%, at least 85%, at least 90%, at least 95%, at least 98%, at least 99%, or 100% sequence identity to SEQ ID NO: 2 or 4. In some examples, the nucleic acid molecule encoding Gsx1 has at least 80%, at least 85%, at least 90%, at least 95%, at least 98%, at least 99%, or 100% sequence identity to SEQ ID NO: 1 or 3, and in some examples is part of a plasmid or vector (such as a lentiviral or AAV vector), and can be operably linked to a promoter, enhancer element, or both.

The cell includes an isolated nucleic acid molecule encoding a Gsx1-CPP fusion protein, wherein a Gsx1 portion of the Gsx1-CPP fusion protein can be encoded by a nucleic acid molecule having at least 80%, at least 85%, at least 90%, at least 95%, at least 98%, at least 99%, or 100% sequence identity to SEQ ID NO: 1 or 3 or encodes a Gsx1 protein having at least 80%, at least 85%, at least 90%, at least 95%, at least 98%, at least 99%, or 100% sequence identity to SEQ ID NO: 2 or 4. The portion of the nucleic acid molecule encoding a CPP portion of the Gsx1-CPP fusion protein can encodes a protein having at least 80%, at least 85%, at least 90%, at least 95%, at least 98%, at least 99%, or 100% sequence identity to any one of SEQ ID NOS: 61-79. In some examples a nucleic acid molecule encoding a Gsx1-CPP fusion protein is part of a plasmid or vector (such as a lentiviral or AAV vector), and can be operably linked to a promoter, enhancer element, or both.

In some examples, following transfection, the recombinant host cells are cultured ex vivo. For example if fibroblast cells are used, they can be cultured in MEF medium [Dulbecco's modified Eagle's medium (DMEM, Hyclone), 10% fetal bovine serum (FBS) (Gibco), 1× Pen/Strep (Gibco), 1× MEM non-essential amino acids (Gibco), and 0.008% (v/v) 2-mercaptoethanol]. For example if NSPCs and ESCs are used, they can be cultured in neuron differentiation medium: DMEM/F12 (Life Technologies) with 1× Pen/Strep, 25 μg/ml insulin, 50 μg/ml Apo-transferrin, 1.28 ng/ml progesterone, 16 ng/ml putrescine, and 0.52 μg/ml sodium selenite; and supplemented with 10 ng/ml recombinant mouse EGF and 10 ng/ml recombinant human bFGF.

The resulting transformed (e.g., recombinant) host cells, which are thus programmed to a neuronal phenotype, can be introduced (e.g., transplanted) into a subject with a neurological disorder, such as a SCI or TBI, or Parkinson's disease, Alzheimer's disease, stroke, ischemia, epilepsy, Huntington's disease, multiple sclerosis, or amyotrophic lateral sclerosis. Thus, such programmed cells can be used to reach such neurological disorders in a subject, such as a human subject.

The disclosure is illustrated by the following non-limiting Examples.

### EXAMPLE 1

### Materials and Methods

This example provides the materials and methods used to obtain the results shown in Examples 2-7.

### Lentivirus Production

Lentiviruses encoding Gsx1 and a reporter RFP (lenti-Gsx1-RFP) and control lentiviruses (encoding only the reporter RFP, lenti-Ctrl-RFP) (ABM® LV465366 and LV084) were generated by transfecting HEK293T cells with a mixture of target vector (lenti-Gsx1-RFP or lenti-Ctrl-RFP), envelope plasmid (pMD2.G/VSVG, Addgene 12259), and 3rd generation packaging plasmids (pMDLg/pRRE, Addgene 12251 and pRSV-Rev, Addgene 12253). HEK293T cells were cultured in high glucose Dulbecco's Modified Eagle Media (DMEM) supplemented with 10% fetal bovine serum (FBS), 1% nonessential amino acid (MEM NEAA 100× Life Technology 11140050), and 1% Glutamax I 100× (Life Technology 35050061). Transfection of the HEK293T (Human Embryonic Kidney) cells was performed when the culture reaches ˜50-60% confluency. Virus containing supernatant was collected at day 2 and day 4 after transfection. Viruses were concentrated by precipitating the virus supernatant by polyethylene glycol 6000 (PEG6000) method (Kutner et al., Nat Protoc 4, 495-505 (2009)). Viral titer was determined by infecting HEK293T cells (Kutner et al., Nat Protoc 4, 495-505 (2009)).

### Animals

Young adult (8-12 weeks) mice were used, under compliance of Institutional Animal Care and Use Committee (IACUC). All animals were housed in an animal care facility with 12-hour light/dark cycle. Mouse under each experimental condition were assigned randomly with equal number of male and female mice when possible.

### Hemisection Spinal Cord Injury and Lentivirus Injection

For hemisection SCI and lentiviral injections, mice were first anesthetized with 5% isoflurane inhalation for 3-4 minutes and then maintained at 2.5% isoflurane for the remainder of the surgery. Next, the skin was disinfected with betadine scrub and 70% ethanol wipes. Laminectomy was performed around T9-T10 t expose the spinal cord. Next, local anesthesia (0.125% Marcain) was applied and dorsal blood vessels were burned using the cauterizer. Then a lateral cut was performed to the left side of the spinal cord and the cut ends at the midline of the spinal cord for hemisection SCI. Immediately after the injury, ˜1-2 μL of virus (1×10⁸ TU/ml) was injected about 1 mm rostral and caudal to lesion epicenter. Virus was injected at about 1 μL/min and the needle was left in place for 2-3 minutes to allow diffusion and prevent leakage or backflow. For the sham animals, skin and muscle were cut to expose the spinal cord. Muscles were sutured and skin was stapled back together. Immediately after surgery, 1 mg/kg Meloxicam, a pain killer, and 50 mg/kg Cefazoline, an antibiotic, were administered subcutaneously.

Animals were divided into the following three groups (3-6 mice/group): 1) sham mice (exposed the spine without injury, Sham); 2) SCI mice with injection of lenti-control-RFP (SCI+Ctrl); and 3) SCI mice with lenti-Gsx1-RFP injection (SCI+Gsx1).

### Behavioral/Locomotion Assessment

Locomotion of each animal was evaluated based on the Basso Mouse Scale (BMS) score by open field test (Basso et al., J Neurotrauma 23:635-659 (2006)). BMS scale ranges from 0 (completely paralyzed) to 9 (normal) locomotion. The BMS score assessment was given by three independent observers who are blinded to the type of treatment after a 2-3 minute observation per animal. The BMS assessment is performed before the injury and then twice a week for up to 8 weeks after injury.

### Tissue Processing

Spinal cord tissues at 3, 7, 14, and 56 days post-injury (DPI) were harvested after intracardial perfusion with 1× phosphate buffer saline (PBS) followed by 4% (w/v) paraformaldehyde (PFA). Spinal cord tissue was then microsurgically dissected and fixed overnight (18-24 hours) in 4% PFA on a rotor. Fixed spinal cord tissue was washed three times with 1× PBS for 30 min and then placed in 30% (w/v) sucrose overnight until tissue sank to the bottom. Next, tissue was cryopreserved by embedding in Tissue-Tek® optimum cutting temperature (O.C.T.) and stored at −80° C. until needed. Sagittal or cross-sections (12 μm thickness) of spinal cord tissues were generated using a cryostat (ThermoScientific).

### Immunohistochemistry

Immunostaining was performed following a previously established protocol with minor modifications (Li et al., Sci Rep 6, 38665 (2016)). Briefly, sections were treated with cold methanol for 10 mins at room temperature for fixation and antigen retrieval. All antibodies were diluted in blocking solution containing 0.05% Triton X-100, 2% donkey serum, 3% bovine serum albumin (BSA), and PBS (1×), pH 7.4. Sections were incubated with primary antibodies (Table 1) overnight at 4° C. and washed three times for 10 min with PBS. Secondary antibodies (Table 1) were then incubated for 1 hour at room temperature and washed three times for 10 min with PBS. For nuclear staining, 4′,6-diamidino-2-phenylindole (DAPI; 200 ng/ml) was added and then samples are washed three times with PBS, and sealed with Cytoseal 20 (ThermoFisher Scientific 8310-4).

TABLE 1
Primary and secondary antibodies used for immunohistochemistry.

| | | Host | | | |
|---|---|---|---|---|---|
| | Vendor, Catalog | Species | Type | RRID | Dilution |
| Primary Antibody | | | | | |
| Gsx1 | Millipore Sigma, | Rabbit | Polyclonal | AB_10667904 | 1:200 |
| | SAB2104632 | | | | |
| Ki67 | Abcam, ab15580 | Rabbit | Polyclonal | AB_443209 | 1:1000 |
| Nestin | Abcam, ab6142 | Mouse | Monoclonal | AB_305313 | 1:200 |
| Caspase3 | Cell Signaling, | Rabbit | Polyclonal | AB_2341188 | 1:300 |
| (cleaved) | 9661S | | | | |
| DCX | Santa Cruz | Goat | Polyclonal | AB_2088491 | 1:100 |
| | Biotechnology, sc-8067 | | | | |
| PDGFRa | Abcam, ab61219 | Rabbit | Polyclonal | AB_2162341 | 1:100 |
| NeuN | Millipore Sigma, | Mouse | Monoclonal | AB_2298772 | 1:300 |
| | MAB377 | | | | |
| GFAP | Millipore Sigma, | Mouse | Monoclonal | AB_477010 | 1:400 |
| | G3893 | | | | |
| Olig2 | Millipore Sigma, | Rabbit | Polyclonal | AB_570666 | 1:500 |
| | AB9610 | | | | |
| vGlut2 | Millipore Sigma, | Guinea | Polyclonal | AB_2665454 | 1:1000 |
| | AB2251-I | Pig | | | |
| ChAT | Millipore Sigma, | Goat | Polyclonal | AB_10603616 | 1:300 |
| | SAB2500236 | | | | |
| GABA | Millipore Sigma, | Rabbit | Polyclonal | AB_477652 | 1:3000 |
| | A-2052 | | | | |
| CS56 | Millipore Sigma, | Mouse | Monoclonal | AB_476879 | 1:200 |
| | C8035 | | | | |
| 5-HT | ImmunoStar | Rabbit | Polyclonal | AB_572262 | 1:10000 |
| 5-HT | ImmunoStar | Goat | Polyclonal | AB_572263 | 1:5000 |
| Secondary Antibody | | | | | |
| Alexa Fluor | Jackson Immuno | — | Polyclonal | AB_2340846 | 1:200 |
| 488 Donkey | Research, | | | | |
| anti Mouse | 715-545-150 | | | | |
| Alexa Fluor | Jackson Immuno | — | Polyclonal | AB_2313584 | 1:200 |
| 488 Donkey | Research, | | | | |
| anti Rabbit | 711-545-152 | | | | |
| Alexa Fluor | Jackson Immuno | — | Polyclonal | AB_2340428 | 1:200 |
| 488 Donkey | Research, | | | | |
| anti Goat | 705-545-003 | | | | |
| Alexa Fluor | Jackson Immuno | — | Polyclonal | AB_2340472 | 1:200 |
| 488 Donkey | Research, | | | | |
| anti Guinea | 706-545-148 | | | | |
| Pig | | | | | |
| Alexa Fluor | Jackson Immuno | — | Polyclonal | AB_2340862 | 1:200 |
| 647 Donkey | Research, | | | | |
| anti Mouse | 715-605-150 | | | | |
| Alexa Fluor | Jackson Immuno | — | Polyclonal | AB_2492288 | 1:200 |
| 647 Donkey | Research, | | | | |
| anti Rabbit | 711-605-152 | | | | |
| Alexa Fluor | Jackson Immuno | — | Polyclonal | AB_2340436 | 1:200 |
| 647 Donkey | Research, | | | | |
| anti Goat | 705-605-003 | | | | |
| Alexa Fluor | Jackson Immuno | — | Polyclonal | AB_2340476 | 1:200 |
| 647 Donkey | Research, | | | | |
| anti Guinea | 706-605-148 | | | | |
| Pig | | | | | |

### Imaging and Image Analysis

At least five sections from each slide/animal were analyzed. Images were captured at the same exposure and threshold and at the same intensity per condition using Zeiss LSM 800 confocal microscope or Zeiss AxioVision Imager A.1. Automatic cell counter in the ImageJ (Rueden et al., BMC Bioinformatics 18:529 (2017), Schneider et al., Nat Methods 9:671-675 (2012)) was used to count the total number of cells. Co-labeling of cell type specific markers with RFP was counted manually. Sample sizes were determined based on power analysis performed from previous experiments. Data are presented as mean±standard error of the mean (SEM). All data were analyzed using GraphPad Prism version 5.0 software for Microsoft Windows. Statistical significance between two condition is calculated by Student's t-test and multi-group comparison is performed using one-way ANOVA, followed by Tukey post-hoc test. P-value of less than 0.05 is considered statistically significant.

### RNA Extraction and Quality Control

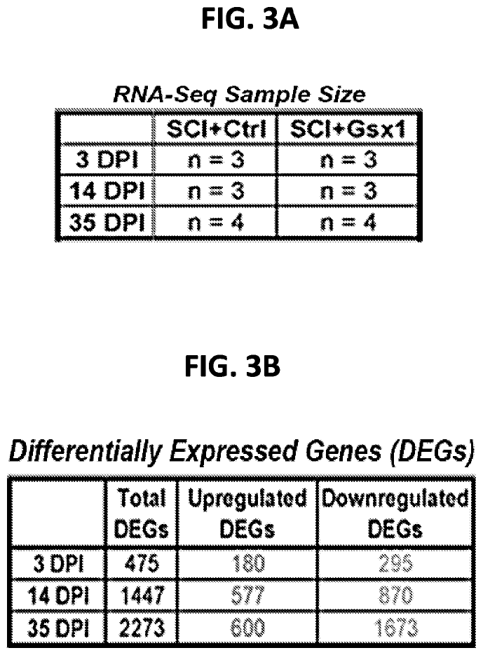

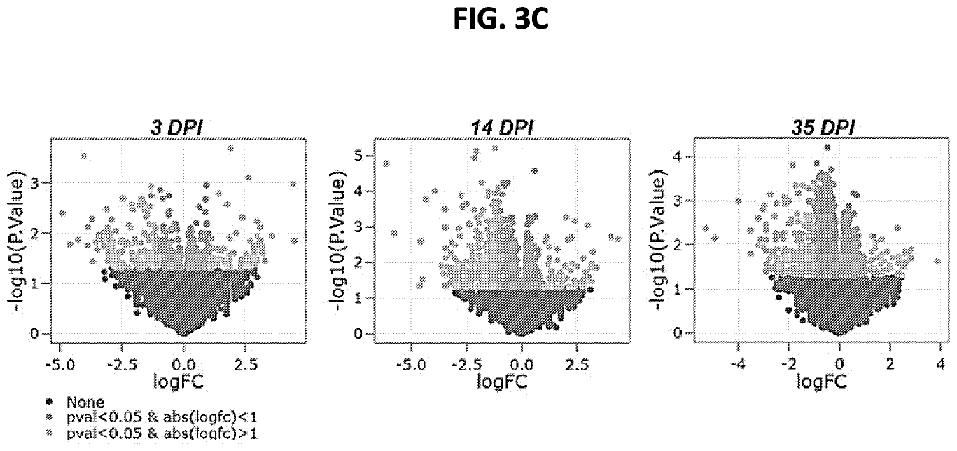

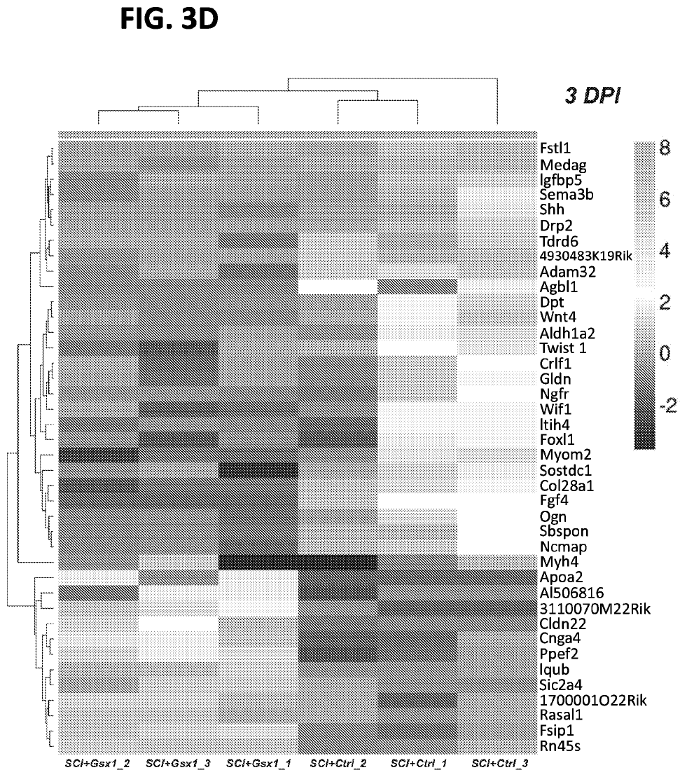

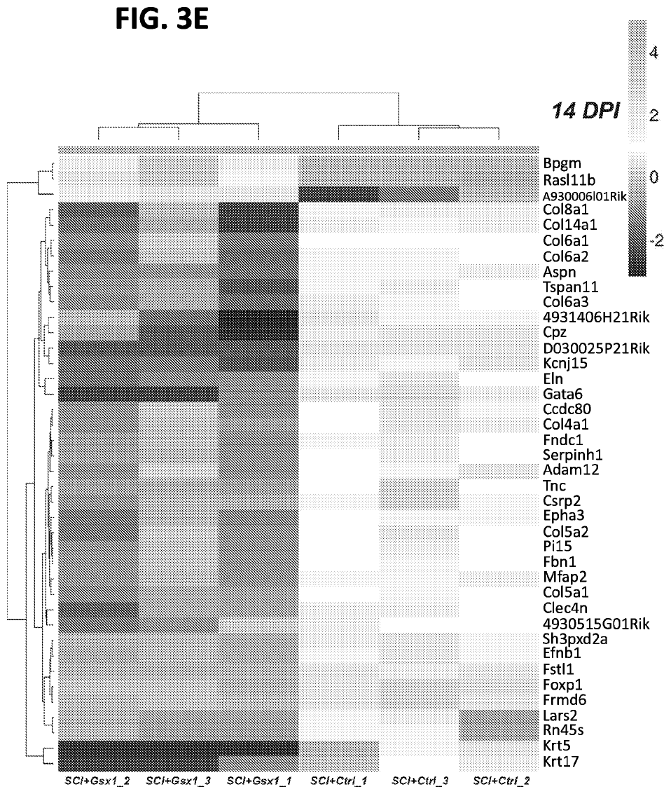

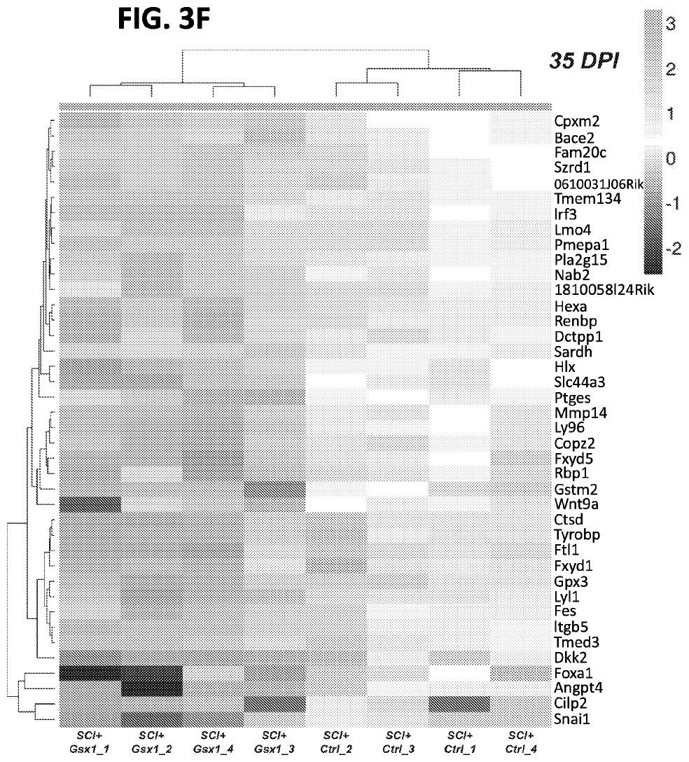

Spinal cord tissues of 3, 14, 35 DPI (n≥3 for each time points; (FIG. 3A)) were dissected out and injured/injected segments (parenchymal segments spanning ˜2-3 mm from each side of the lesion) were rapidly snap frozen in liquid nitrogen. Total RNA was isolated from spinal cord tissues using RNeasy Lipid Tissue Mini kit (Qiagen, #74804) following the manufacture's protocol. The concentration of the total RNA was determined using Qubit RNA BR Assay Kit (Life Technologies) and quality of the total RNA was determined using the RNA 6000 Nano chip on the 2100 Bioanalyzer automated electrophoresis system (Agilent Technologies).

### Library Preparation and RNA-Sequencing

Library preparation and RNA-sequencing were performed by Admera Health (South Plainfield, N.J.). Total RNA was used for library preparation of each sample, which was subsequently bar-coded and prepared according to manufacturer's instructions (Illumina). The libraries were prepared using an Illumina MiSeq paired-end kit and sequenced as paired-end, 2×150 bp on the Illumina MiSeq. The sequencing run was performed according to the manufacturer's instructions and generated a total of 40 million reads per sample.

### RNA-Seq Data Analysis and Pathway Analysis

After a quality check of the raw fastq files using the FastQC (www.bioinformatics.babraham.ac.uk/projects/fastqc/), all sequences were aligned to the mouse reference genome, mm10, with STAR version 2.0 (Dobin et al., Bioinformatics 29:15-21 (2013)). The raw read counts were generated using HTSeq (version 0.6.0) (Anders et al., Bioinformatics 31:166-169 (2015)) . The DESeq2 (Love et al., Genome Biol 15:550 (2014); Anders & Huber, Genome Biol 11:R106 (2010)), a R/Bioconductor package, was used to normalize the counts and call differential gene expression on counts matrix generated by HTSeq. Differentially expressed transcripts/genes between Gsx1 treatment and control groups, were defined by statistical significance (p-value) and biological relevance (fold change). Downstream pathway analysis was carried out using QIAGEN's Ingenuity Pathway Analysis (IPA, QIAGEN Redwood City, www.qiagenbioinformatics.com/products/ingenuity-pathway-analysis).

Gene expression of the box blot is generated from count matrix from the HTSeq using START (kcvi.shinyapps.io/START/) and with edgeR algorithm. Each dot on the box plot represent one biological sample.

Quantitative Real-Time PCR (qPCR) Analysis

Complementary DNA (cDNA) was synthesized from total RNA using SuperScript III First-Strand Synthesis System (Invitrogen, 18080051) using the manufacture's protocol. qPCR was performed with Power SYBR™ Green PCR Master Mix and gene specific primers (Table 2) using StepOnePlus Real-Time PCR system (Applied Biosystem). GAPDH is used as a reference housekeeping gene. The Levak method is used to calculate the fold change, by normalizing it to the Sham.

TABLE 2
Forward and reverse primers (5'→3') used for RT-qPCR analysis

| | | SEQ ID | | SEQ ID |
|---|---|---|---|---|
| Gene | Forward (5'→3') | NO | Reverse (5'→3') | NO |
| Gsxl | CTTCCCTCCCTTCGGATCG | 5 | GTCCACAGAGATGCAGTGAAA | 33 |
| Cd68 | GGACCCACAACTGTCACTCAT | 6 | AAGCCCCACTTTAGCTTTACC | 34 |
| Itgam | ATGGACGCTGATGGCAATACC | 7 | TCCCCATTCACGTCTCCCA | 35 |
| Cd86 | TGTTTCCGTGGAGACGCAAG | 8 | TTGAGCCTTTGTAAATGGGCA | 36 |
| Il lb | GCAACTGTTCCTGAACTCAACT | 9 | ATCTTTTGGGGTCCGTCAACT | 37 |
| Tnf | CCTGTAGCCCACGTCGTAG | 10 | GGGAGTAGACAAGGTACAACCC | 38 |
| Ki67 | ATCATTGACCGCTCCTTTAGGT | 11 | GCTCGCCTTGATGGTTCCT | 39 |
| (Mki67) | | | | |
| Nestin | CCCTGAAGTCGAGGAGCTG | 12 | CTGCTGCACCTCTAAGCGA | 40 |
| NeuN | AACCACGAACTCCACCCTTC | 13 | GACCTCAATTTTCCGTCCCTC | 41 |
| (Hrnbp3) | | | | |
| vGlut | TGGAAAATCCCTCGGACAGAT | 14 | CATAGCGGAGCCTTCTTCTCA | 42 |
| (S1c17a6) | | | | |
| Th | GTCTCAGAGCAGGATACCAAGC | 15 | CTCTCCTCGAATACCACAGCC | 43 |
| Tphl | AACAAAGACCATTCCTCCGAAAG | 16 | TGTAACAGGCTCACATGATTCTC | 44 |
| Chat | CCATTGTGAAGCGGTTTGGG | 17 | GCCAGGCGGTTGTTTAGATACA | 45 |
| Gfap | CGGAGACGCATCACCTCTG | 18 | AGGGAGTGGAGGAGTCATTCG | 46 |
| Lcn2 | GCAGGTGGTACGTTGTGGG | 19 | CTCTTGTAGCTCATAGATGGTGC | 47 |
| Serpina3n | ATTTGTCCCAATGTCTGCGAA | 20 | TGGCTATCTTGGCTATAAAGGGG | 48 |
| Notchl | CCCTTGCTCTGCCTAACGC | 21 | GGAGTCCTGGCATCGTTGG | 49 |
| Nrarp | AAGCTGTTGGTCAAGTTCGGA | 22 | CGCACACCGAGGTAGTTGG | 50 |
| Jagl | CCTCGGGTCAGTTTGAGCTG | 23 | CCTTGAGGCACACTTTGAAGTA | 51 |
| Jag2 | CACTGTCCGTCAGGATGGAAC | 24 | TAGCCGCCAATCAGGTTTTTG | 52 |
| D111 | CCCATCCGATTCCCCTTCG | 25 | GGTTTTCTGTTGCGAGGTCATC | 53 |
| Hes' | TCAGCGAGTGCATGAACGAG | 26 | CATGGCGTTGATCTGGGTCA | 54 |
| Cdhl | CAGGTCTCCTCATGGCTTTGC | 27 | CTTCCGAAAAGAAGGCTGTCC | 55 |
| Bmprla | TGCAAGGATTCACCGAAAGC | 28 | TGCCATCAAAGAACGGACCTAT | 56 |
| Col6a2 | GCTCCTGATTGGGGGACTCT | 29 | CCAACACGAAATACACGTTGAC | 57 |
| Ctnnal | AAGTCTGGAGATTAGGACTCTGG | 30 | ACGGCCTCTCTTTTTATTAGACG | 58 |
| Ntngl | TGCTAAACACAGTCATTTGCGT | 31 | GCACACATTCTCATCGTCCAG | 59 |
| Synl | AGCTCAACAAATCCCAGTCTCT | 32 | CGGATGGTCTCAGCTTTCAC | 60 |

### EXAMPLE 2

### Overview of Methods

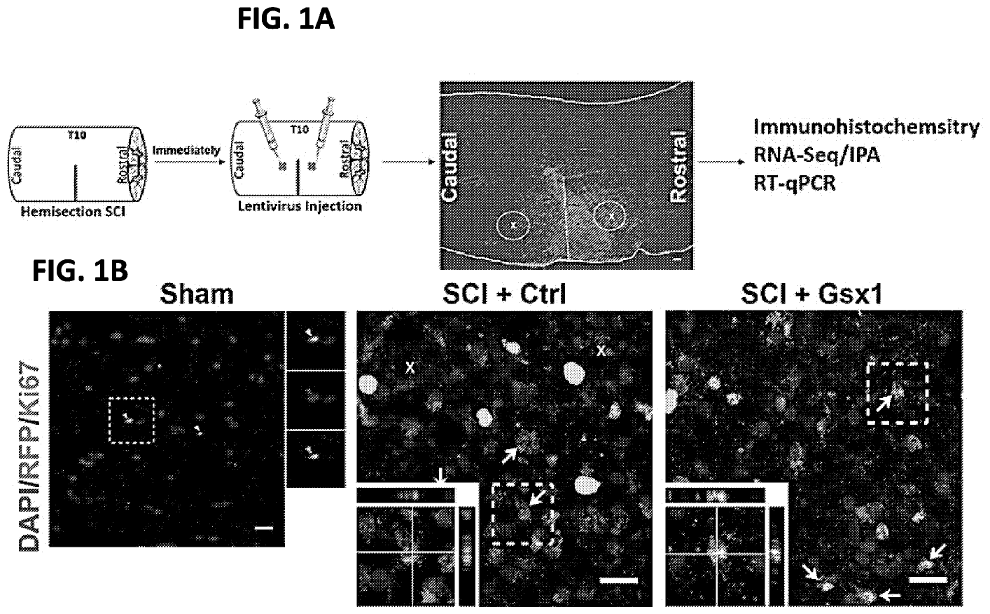

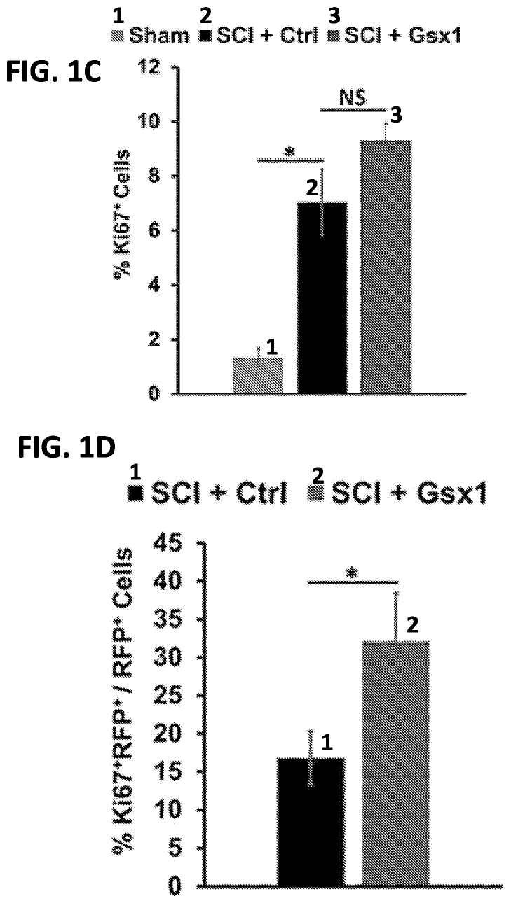

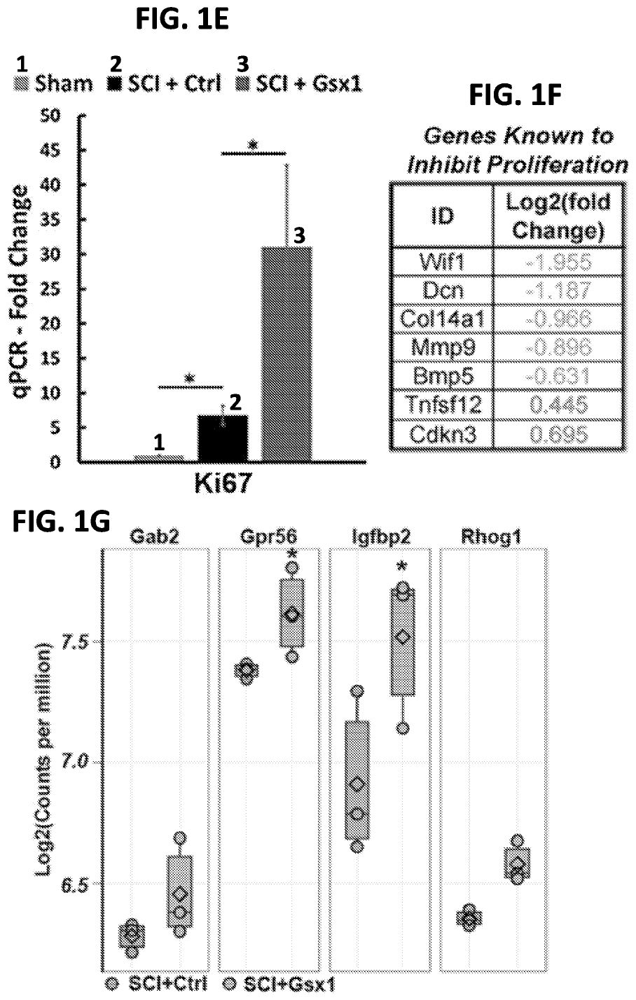

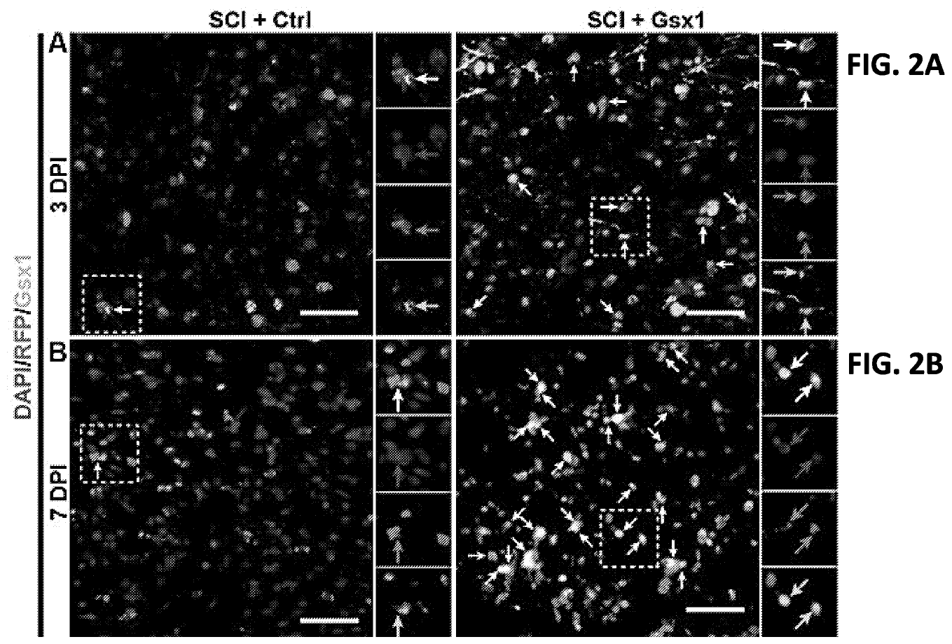

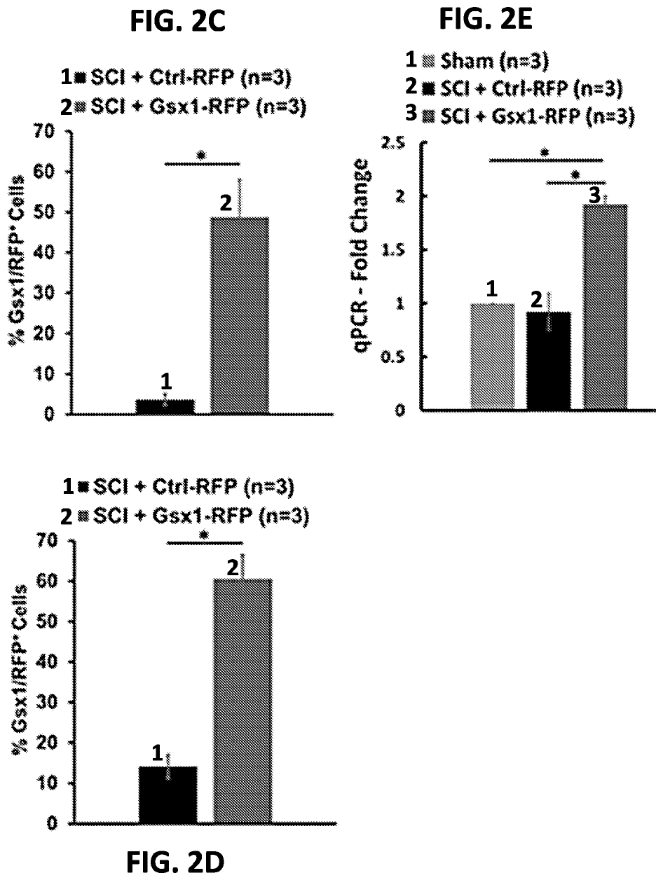

To determine if ectopic expression of Gsx1 in the adult spinal cord after SCI will promote NSPC activation and neurogenesis, a lateral hemisection from the midline to the left side of the spinal cord at the thoracic (T) 10 level was performed. The completeness and consistency of the lateral hemisection SCI was confirmed by the observation of paralysis in the left hind limb. Immediately after the SCI, 1 μL/site of lentivirus (1×10⁸ TU/ml) encoding Gsx1 and a reporter red fluorescent protein (RFP) (lenti-Gsx1-RFP) was injected into the injured spinal cord, approximately 1 mm rostral and caudal to the injury site (FIG. 1A). Lentivirus encoding only the RFP reporter (lenti-Ctrl-RFP) is used as a control. Animals were randomly assigned to the following three groups (3-6 mice/group): 1) sham mice (exposed the spine without injury, Sham); 2) SCI mice with injection of lenti-Ctrl-RFP (SCI+Ctrl); and 3) SCI mice with lenti-Gsx1-RFP injection (SCI+Gsx1). It was first confirmed that the lentivirus-mediated Gsx1 expression in the spinal cord tissue at 3 days-post injury (DPI) and 7 DPI by immunohistochemistry and RT-qPCR at 3 DPI (FIGS. 2A-2E). Compared to the control, Gsx1 treatment significantly increased the percentage of virally transduced RFP+cells with Gsx1 expression.

### EXAMPLE 3

### Gsx1 Treatment Increases Cell Proliferation in Injured Spinal Cord

SCI increases cell proliferation at the lesion site. To determine the effect of the Gsx1 treatment on cell proliferation at 3 days-post injury (DPI), the expression of the cell proliferation marker Ki67 was examined by immunohistochemistry followed by confocal imaging analysis (FIG. 1B). The RFP+ and Ki67+ cells were found to be located around the injection sites. The percentage of the Ki67+ cells among DAPI+ cells around the injury/injection areas in the following 3 control and experimental groups: Sham (n=3), SCI+Ctrl (n=6), and SCI+Gsx1 (n=6) (FIG. 1C). A significant increase in the percentage of Ki67+ cells was observed in both injury groups that received viral injection compared to the Sham mice, with the highest increase in the SCI+Gsx1 group (FIG. 1C). In addition, a significantly higher percentage of Ki67+/RFP+ co-labeled cells among RFP+ cells were found in the mice with lenti-Gsx1-RFP injections compared to mice that received lenti-Ctrl-RFP injections (FIG. 1D). Furthermore, the increase in Ki67 mRNA expression was validated by RT-qPCR analysis. Gsx1 treatment induces a significantly higher level of Ki67 mRNA level (˜4-fold; FIG. 1E) in the SCI+Gsx1 group compared to the SCI+Ctrl group. These results indicate that Gsx1 treatment promotes cell proliferation in the adult injured spinal cord.

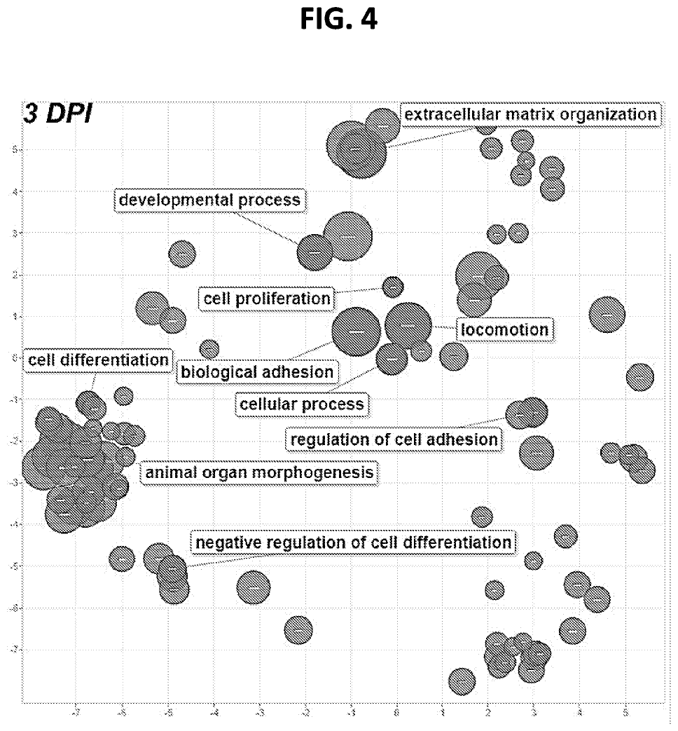

The effect of Gsx1 on promoting cell proliferation may be due to its regulation of genes associated with cell proliferation. A genome-wide transcriptome profiling using RNA-Seq was performed (FIGS. 3A-3C) and followed by pathway analysis by IPA (QIAGEN Inc., www.qiagenbioinformatics.com/products/ingenuitypathway-analysis, Kramer et al., Bioinformatics 30, 523-530 (2014)). RNA-seq analysis identified 475, 1447, and 3946 differentially expressed genes (DEGs) at 3, 14 and 35 DPI, respectively (FIGS. 3B-3C). The top 40 DEGs (Table 3) were shown in heatmap at 3 DPI (FIG. 3D), 14 DPI (FIG. 3E), and 35 DPI (FIG. 3F). Further gene ontology enrichment analysis of the 475 DEGs using REVIGO (Supek et al., PLoS One 6, e21800 (2011)) revealed that cell proliferation is one of the key biological processes being affected with lenti-Gsx1 treatment (FIG. 4). In particular, Gsx1 treatment induced a downregulation of the genes that inhibit cell proliferation (e.g., Wif1, Dcn, Mmp9; FIG. 1F) and upregulation of the genes that promote cell proliferation (e.g., Gab, Gpr56, Igfbp2, Rhog; FIG. 1G). These data confirm that Gsx1 treatment increases cell proliferation in mice with SCI at the acute phase at 3 DPI.

TABLE 3
Top 40 differentially expressed genes. Top 20 significantly (p < 0.05) expressed
upregulated and top 20 downregulated genes determined by RNA-Seq analysis from SCI + Ctrl and
SC + Gsx1 treatment at 3 DPI, 14 DPI, and at 35 DPI. n = 3 at 3 DPI and 14 DPI; n = 4 at 35 DPI.

| 3 DPI | | 14 DPI | | 35 DPI | |
|---|---|---|---|---|---|
| | Log2(Fold | | Log2(Fold | | Log2(Fold |
| ID | Change) | ID | Change) | ID | Change) |

Top 20 Upregulated Genes

| | | | | | |
|---|---|---|---|---|---|
| Ppef2 | 1.7456 | Bpgm | 0.6457 | 9330175M20Rik | 0.9304 |
| Cnga4 | 1.5203 | Snca | 0.6409 | Gm2897 | 0.9209 |
| Tmem51as1 | 1.4642 | Fam46c | 0.5624 | 4930525G20Rik | 0.8941 |
| Al506816 | 1.3532 | Gjc2 | 0.5451 | Zfp819 | 0.8928 |
| Cdsn | 1.3006 | Nkx2-9 | 0.5346 | Ripply2 | 0.8629 |
| G530011O06Rik | 1.2743 | Ppp1r14a | 0.5226 | Peg12 | 0.8311 |
| Fsip1 | 1.2560 | Rasl11b | 0.5223 | Mum1l1 | 0.8199 |
| 3110070M22Rik | 1.2345 | Prr18 | 0.5131 | 1500015L24Rik | 0.8142 |
| Fbxw10 | 1.2303 | Klk6 | 0.5103 | 1110015O18Rik | 0.8049 |
| Galr3 | 1.2074 | Ptgs1 | 0.5076 | 4930441O14Rik | 0.8042 |
| Rn45s | 1.2041 | Ptp4a1 | 0.4919 | Sox14 | 0.7864 |
| C130026l21Rik | 1.1821 | S100b | 0.4672 | Zfp804b | 0.7683 |
| Erv3 | 1.1741 | Bin2 | 0.4563 | Mir331 | 0.7637 |
| Afp | 1.1694 | Tmem88b | 0.4517 | Cacna1f | 0.7612 |
| A330048O09Rik | 1.1366 | Mbp | 0.4422 | Fgf5 | 0.7601 |
| Xlr3a | 1.1299 | Atp10b | 0.4393 | Mipol1 | 0.7477 |
| 1700001O22Rik | 1.1218 | Isg20 | 0.4377 | Mir149 | 0.7471 |
| Apoa2 | 1.1206 | Al848285 | 0.4364 | Tmem232 | 0.7392 |
| Mir466i | 1.1092 | Arhgef37 | 0.4320 | Gpr88 | 0.7370 |
| Crybb1 | 1.0996 | Plp1 | 0.4288 | 6430584L05Rik | 0.7369 |

Top 20 Downregulated Genes

| | | | | | |
|---|---|---|---|---|---|
| Col28a1 | −2.4223 | Col1a1 | −2.0110 | Snai1 | −1.4631 |
| Ogn | −2.0659 | Aspn | −1.8378 | Wfdc17 | −1.4354 |
| Wif1 | −1.9547 | Col1a2 | −1.7949 | Dkk2 | −1.4094 |
| Sbspon | −1.7291 | Col6a3 | −1.6645 | Cilp2 | −1.3829 |
| Itih4 | −1.6936 | Col5a1 | −1.4541 | H19 | −1.3015 |
| Twist1 | −1.6811 | Kcnj15 | −1.4052 | Asgr2 | −1.2978 |
| Sostdc1 | −1.6651 | Mfap5 | −1.3046 | Atp6v0a4 | −1.2820 |
| Plekha4 | −1.6475 | Thbs1 | −1.2104 | Foxa1 | −1.2655 |
| Ncmap | −1.6447 | Serpinh1 | −1.1806 | Apoc2 | −1.2628 |
| Aqp1 | −1.6233 | Ppic | −1.1093 | Angpt4 | −1.2556 |
| Gldn | −1.6205 | Loxl1 | −1.0945 | Fam180a | −1.2413 |
| Prx | −1.6059 | Tnc | −1.0594 | Cd8b1 | −1.2400 |
| Wnt4 | −1.5861 | Ltbp2 | −1.0186 | Twist1 | −1.2251 |
| Cdh1 | −1.5587 | Cpz | −1.0108 | Pi16 | −1.2225 |
| Ngfr | −1.5471 | Scara5 | −0.9871 | Gstm2 | −1.2204 |
| Slc43a1 | −1.5467 | Scara3 | −0.9867 | Wnt9a | −1.1770 |
| Foxd1 | −1.5382 | Rcn3 | −0.9815 | Dpt | −1.1667 |
| Kcnj13 | −1.5141 | Mrc2 | −0.9796 | Col6a2 | −1.1481 |
| Crlf1 | −1.4928 | Sh3pxd2a | −0.9693 | Gpnmb | −1.1465 |
| Dpt | −1.4878 | Tspan11 | −0.9674 | Ms4a7 | −1.1420 |

### EXAMPLE 4

### Gsx1 Treatment ppromotes NSPC Activation After SCI

In the adult spinal cord, NSPCs exist in quiescent states under normal conditions, but become activated after injury. To investigate the effect of Gsx1 treatment on the activation of NSPCs, the expression of NSPC markers (Nestin and Notch1) in the injured spinal cord at 3 DPI via immunohistochemistry and confocal imaging analysis was examined.

The RFP+ and Nestin+ cells were found around the injection sites (FIG. 5A). A significant increase in the percentage of Nestin+ cells was observed among DAPI+ cells at the lesion site in the injury groups that received viral injection (SCI+Ctrl and SCI+Gsx1) compared to the Sham mice, with the highest increase in the SCI+Gsx1 group (FIG. 5B). Further, a significantly higher percentage of Nestin+/RFP+ co-labeled cells among RFP+ cells were found in SCI+Gsx1 group compared to SCI+Ctrl group (FIG. 5C). In addition, RT-qPCR analysis confirmed that Gsx1 treatment significantly increased Nestin mRNA expression (FIG. 5D).

To elucidate the induction in NSPC activation after Gsx1 treatment, Gsx1-induced signaling pathways in the SCI+Ctrl (n=3) and SCI+Gsx1 (n=3) groups at 3 DPI was investigated through RNA-Seq, IPA, and gene ontology analysis using REVIGO. It was observed that negative regulation of cell differentiation was one of the key biological processes affected after lenti-Gsx1 treatment (FIG. 4). IPA analysis showed a significant upregulation of the genes involved in the Notch signaling pathway (e.g., Hes7 and Rbpj) and a downregulation of the transcription repressor gene Hes1 (FIG. 5E). Immunohistochemistry analysis using anti-Notch1 antibody on sagittal sections of the spinal cord tissues at 3 DPI showed a significant increase in the number of Notch1+/RFP+ cells in Gsx1 treatment compared to the control (FIGS. 5F and 5G). The RT-qPCR analysis confirmed a significant increase in the mRNA expression of Notch1 and Jag1 after SCI (SCI+Ctrl and SCI+Gsx1) compared to the Sham group and a further increase in Notch1 mRNA level in lenti-Gsx1 treatment compared to the control (FIG. 5H).

Nrarp, a negative regulator of the Notch signaling pathway that physically interacts with Notch intracellular domain (NICD) and blocks Notch transcription, was downregulated in the SCI+Gsx1 group (FIG. 5H). Furthermore, Gsx1 treatment decreased expression of the Del1 and Hes1 gene (FIG. 5H). Del1 promotes stem cell differentiation to glial lineage, while Hes1 is a transcriptional repressor. The expression of Hes1 results in premature neuronal differentiation.

In addition, an increase in genes associated with activation of Nanog signaling pathway (e.g., Akt2, Map2k2, Pik3cd, Pik3cg, and Rap2b) was observed (FIG. SI). Nanog is an essential pathway in embryonic stem cells (ESCs) and the Nanog gene is commonly expressed in NSPCs. In contrast, the expression of genes in Notch/Nanog signaling pathways was not detected by 35 DPI.

Thus, these results indicate that Gsx1-induced transient upregulation of Notch/Nanog signaling pathways play a role in endogenous NSPC activation in the injured spinal cord.

### EXAMPLE 5

### Induction of Neurogenesis in the Injured Spinal Cord

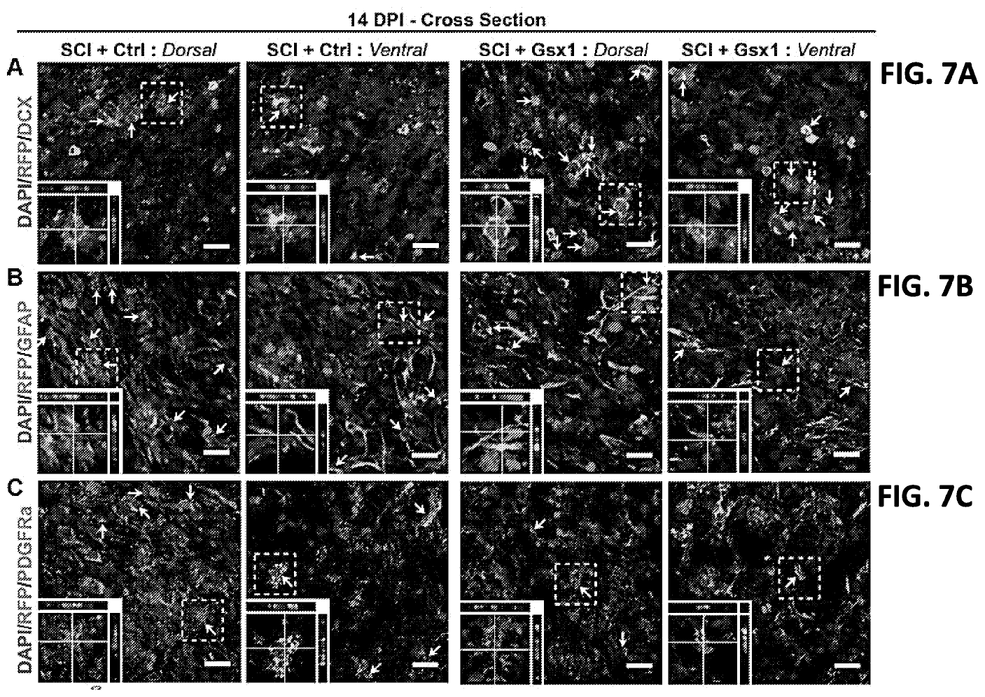

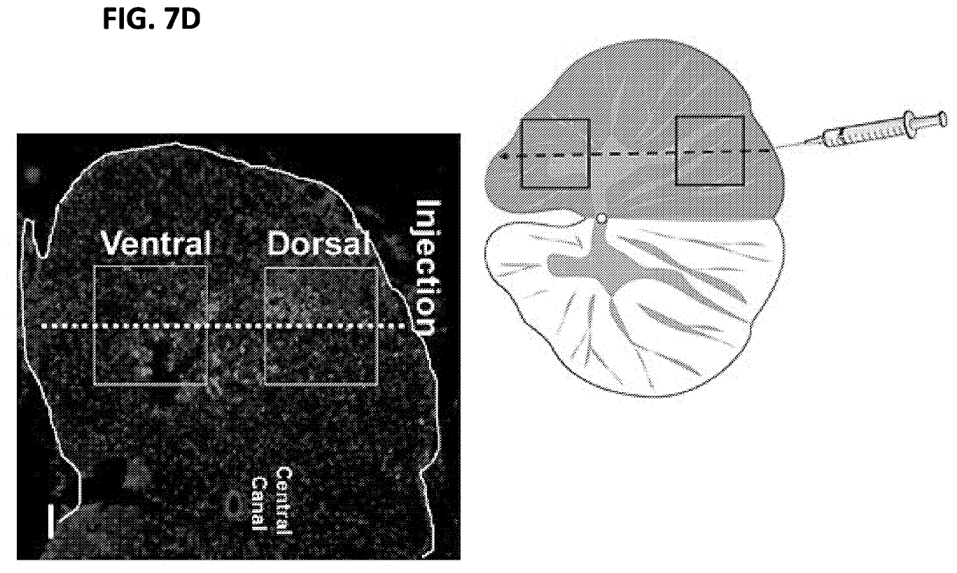

In the adult spinal cord, injury-activated NSPCs mostly generate astrocytes and oligodendrocytes. To determine whether Gsx1 treatment alters cell fate determination in NSPC lineage development, sagittal section (FIGS. 6A-6D) and cross-section (FIGS. 7A-7D) of spinal cord tissues at 14 DPI were examined with an early neuronal marker doublecortin (DCX) (FIG. 6A), an astrocyte marker GFAP (FIG. 6B), and an oligodendrocyte progenitor marker PDGFRa (FIG. 6C) in the SCI+Ctrl (n=6) and SCI+Gsx1 (n=6) groups. DCX is mostly expressed in neuroblasts and immature neurons and is associated with adult neurogenesis, but not reactive gliosis. Compared to the SCI+Ctrl group, Gsx1 treatment significantly increased the percentage of DCX+/RFP+ co-labeled cells and decreased the percentage of GFAP+/RFP+ and PDGFRa+/RFP+ co-labeled cells among RFP+cells (FIG. 6D). No noticeable difference in the number of DCX+, GFAP+, and PDGFR+ cells was found between the dorsal and ventral region of the spinal cord at 14 DPI within SCI+Ctrl and SCI+Gsx1 groups (FIGS. 7A-7D).

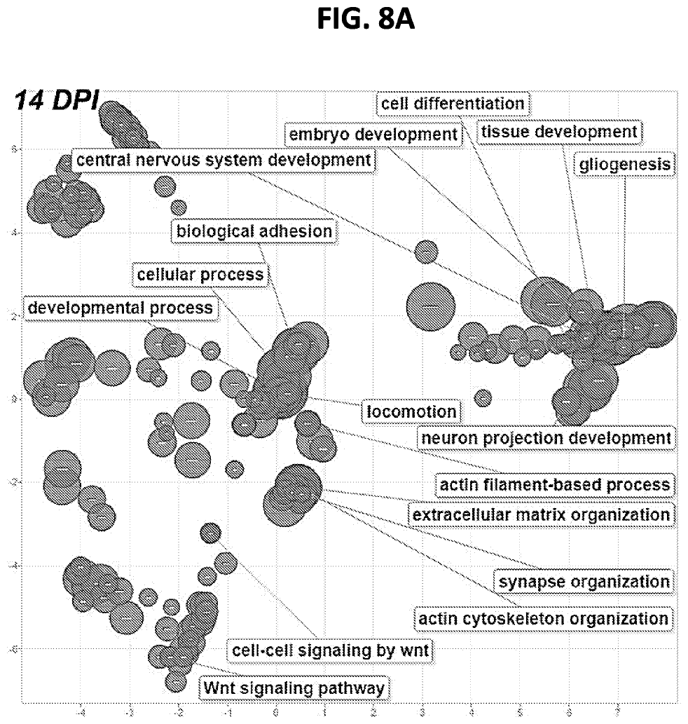

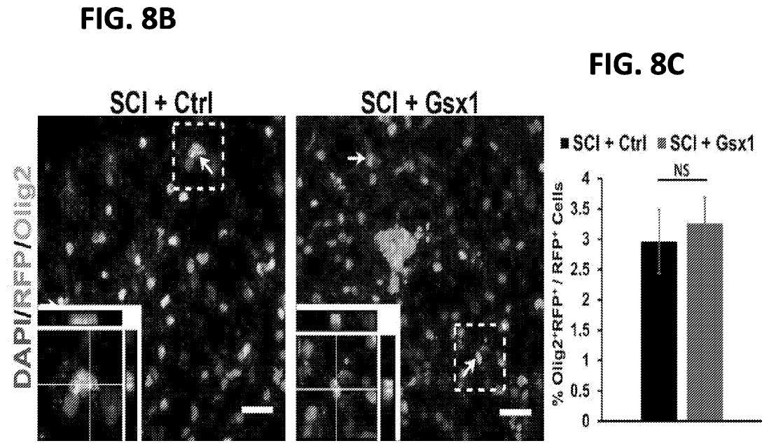

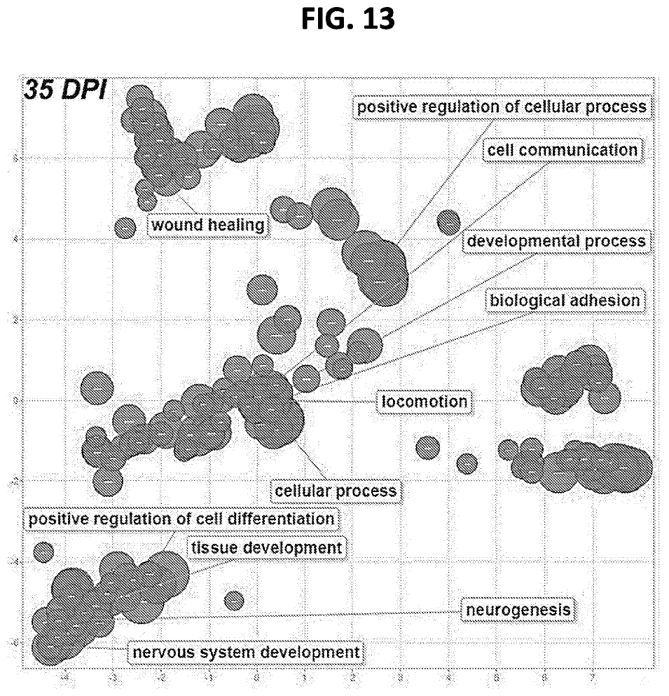

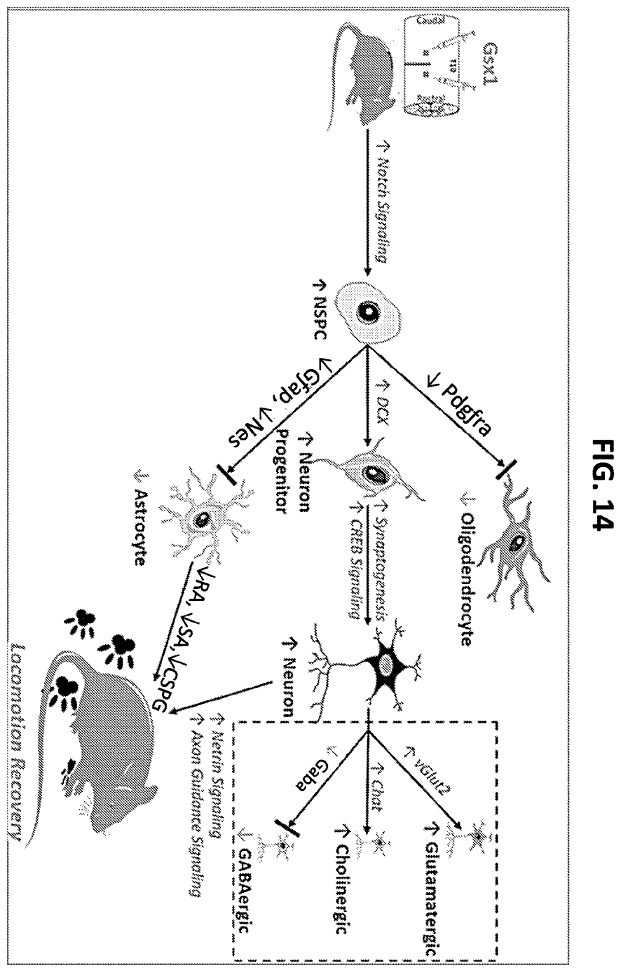

Gene ontology analysis of 1447 DEGs (identified by RNA-SEQ; FIGS. 3B-3C) at 14 DPI revealed that enrichment of DEGs involved in cell differentiation, neuron projection development, synapse organization, and central nervous system development as some of the key biological processes being affected after Gsx1 treatment (FIG. 8A). Additionally, REVIGO analysis revealed that neurogenesis and nervous system development as few of the key biological processes being affected upon Gsx1 treatment (FIG. 13). The 2273 DEGs at 35 DPI (FIGS. 3B-3C) shared a similar trend in NSPC lineage classification as those of the 14 DPI, e.g., an upregulation in DCX (FIG. 6E), downregulation in GFAP (FIG. 6F) and PDGFRa (FIG. 6G) in the SCI+Gsx1 group when compared to the SCI+Ctrl group. However, there was not significant (NS) difference in Olig2+/RFP+ cells between the control and Gsx1 treatment groups at 56 DPI (Supplementary FIGS. 8B-8C).

These results indicate that Gsx1 treatment induces NSPC differentiation towards neuronal over glial lineage during the chronic phase of SCI.

### EXAMPLE 6

### Gsx1 Treatment Increases the Number of Specific Subtypes of Interneurons

The specific subtypes of mature neurons induced by Gsx1 treatment were identified. Sagittal sections of spinal cord tissues at 56 DPI were stained with a mature neuronal marker NeuN (FIG. 9A), a cholinergic neuronal marker ChAT (FIG. 9B), a glutamatergic interneuron marker vGlut2 (FIG. 9C), and a GABAergic interneuron marker GABA (FIG. 9D). A significant increase in the percentage of NeuN+, ChAT+, and vGlut2+ cells, and a decrease in GABA+ cells among RFP+ cells in the SCI+Gsx1 group (n=6), was observed, as compared to the SCI+Ctrl group (n=6) (FIG. 9E). Further, RT-qPCR analysis of spinal cord tissues from Sham (n=4), SCI+Ctrl (n=4), and SCI+Gsx1 (n=4) groups at 35 DPI when induced cells reach maturity showed a significantly increased expression of vGlut (or Slc17a6) and Chat, accompanied by a slightly increased mRNA expression of NeuN (or Hrnbp3) (FIG. 9F).

These results indicate that Gsx1 treatment preferentially increased the number of glutamatergic and cholinergic interneurons and decreased the number of GABAergic interneurons.

### EXAMPLE 7

### Gsx1 Treatment Reduces Glial Scar Formation

SCI causes activation of microglia and astrocytes, which leads to reactive astrogliosis and glial scar formation. Glial scar is mostly composed of reactive astrocytes (RA), non-neuronal cells (e.g., pericytes and meningeal cells), and proteoglycan-rich extracellular matrix (ECM). Activated astrocytes secrete chondroitin sulfate proteoglycan (CSPG), which constitutes the major component of the glial scar.

To investigate the role of Gsx1 in astrogliosis and scar formation, the mRNA expression level of two marker genes, GFAP and Serpina3n, were measured to detect reactive astrocytes (RA) involved in astrogliosis and scar formation 53 by RT-qPCR analysis. SCI significantly increased the mRNA expression levels of GFAP and Serpina3n in the SCI+Ctrl (n=6) and SCI+Gsx1 (n=6) mice compared to the Sham mice (n=4) at 3 DPI (FIG. 10A) and 35 DPI (FIG. 10B), confirming that injury caused astrogliosis and scar formation. In contrast, Gsx1 treatment significantly reduced the mRNA expression of GFAP at 3DPI (FIG. 10A) and Serpina3n in the injured spinal cord at 35 DPI (FIG. 10B). RNA-Seq analysis revealed that the expression of genes associated with RA (e.g., Mmp13, Mmp2, Nes, Axin2, Slit2, Plaur, and Ctnnb1), scar-forming astrocytes (SA) (e.g., Slit2 and Sox9), and both RA+SA (e.g., Gfap and Vim) 51 were downregulated at 14 DPI and 35 DPI (FIGS. 10C-10E).

GFAP (FIGS. 10F-10G) and CSPG (FIGS. 10H-10I) protein expression levels were determined by immunohistochemistry analysis using anti-GFAP and anti-CS56 antibodies, respectively. A baseline level of GFAP (FIG. 10F) but no detectable level of CSPG expression (FIG. 10H) were observed in the Sham group. In contrast, injury induced a higher protein level of GFAP (FIG. 10G) and CS56 (FIG. 10I) in the two SCI groups (i.e., SCI+Ctrl and SCI+Gsx1). Importantly, Gsx1 treatment greatly reduced GFAP+ and CS56+ immunostained area around the lesion site in the SCI+Gsx1 group (FIGS. 10G, 10I).

These results indicate that Gsx1 treatment reduces RA and SA, leading to attenuation of glial scar formation after SCI.

### EXAMPLE 8

### Gsx1 Treatment Improves Locomotor Function After SCI

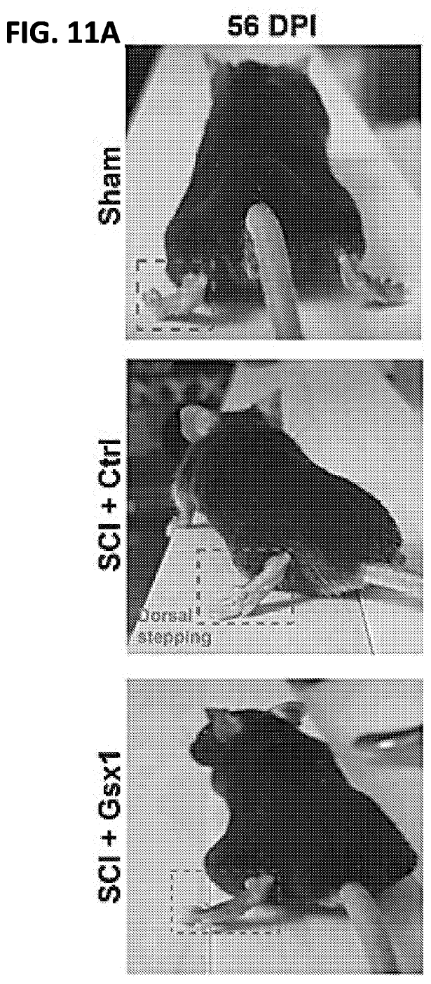

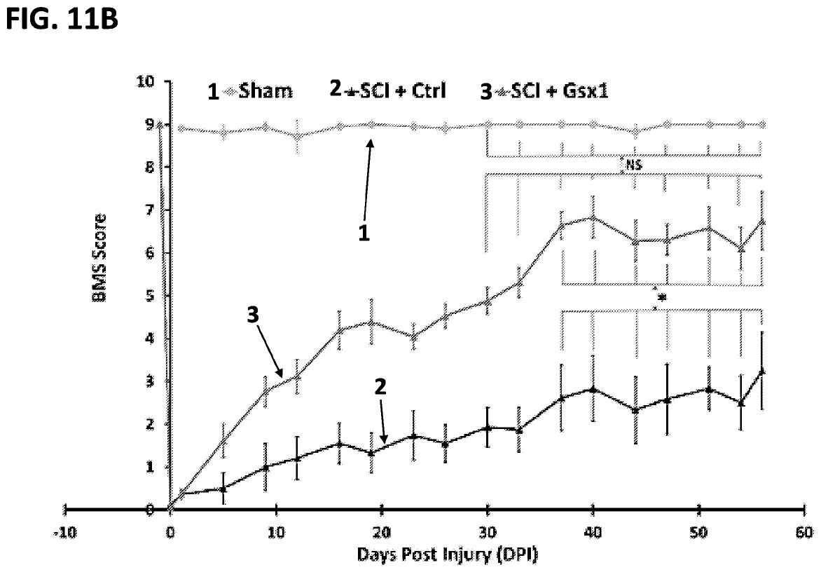

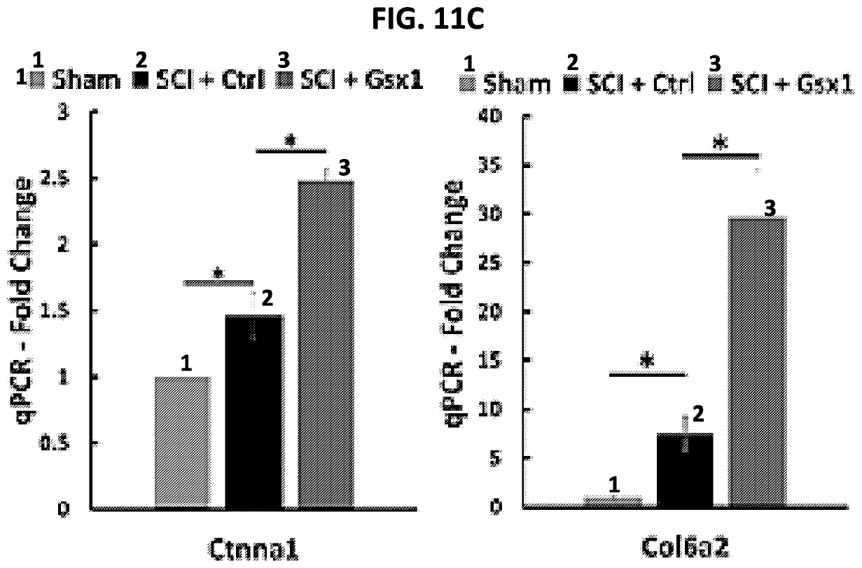

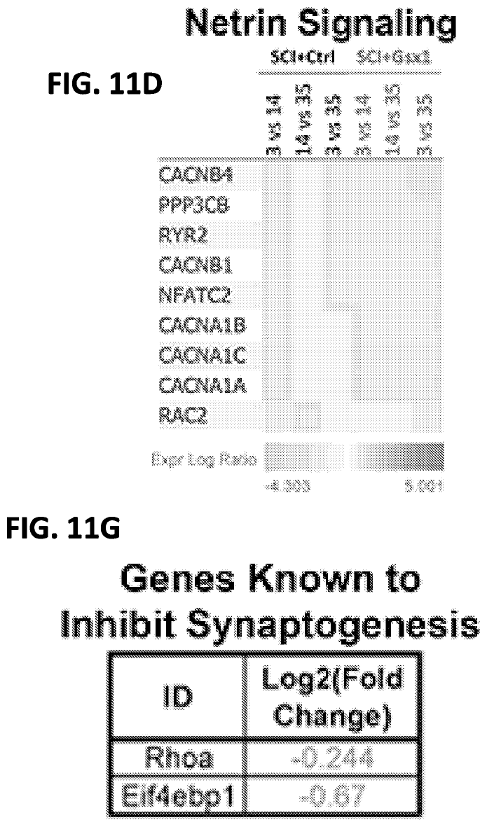

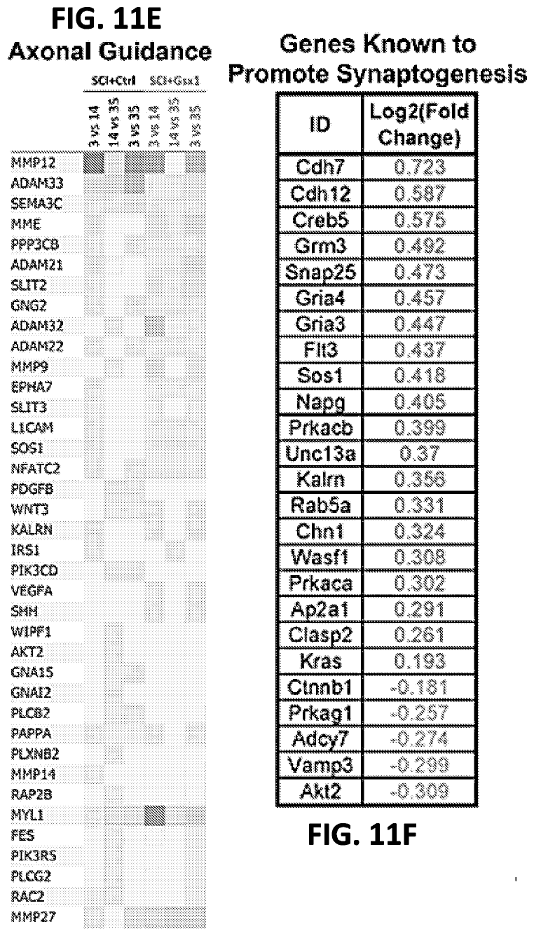

All mice with a lateral hemisection SCI at the T10 level exhibited paralysis in the left hindlimb after injury (FIG. 11A). To demonstrate the effect of Gsx1 treatment on functional recovery, locomotor behavior was assessed using an established open-field locomotion test and a BMS score scale (Basso et al., J Neurotrauma 23:635-659 (2006)) starting from the day before the injury (−1 DPI) to 56 DPI (8 weeks after SCI). For each mouse, a BMS score was assigned double-blindly by three observers. BMS score ranges from 0 (complete paralysis and no ankle movement) to 9 (normal walking). The Sham animals displayed a normal locomotor behavior with BMS score remained at ˜9 from −1 to 56 DPI (FIG. 11B). Mice in the injury groups (SCI+Ctrl and SCI+Gsx1) exhibited paralysis in the left hindlimb with a BMS score of 0 on the day of hemisection injury (0 DPI) (FIGS. 11A-11B and videos found at stemcell.rutgers.edu/Treatment.mov; stemcell.rutgers.edu/Control.mov; stemcell.rutgers.edu/Sham.mov), confirming the success of inducing lateral hemisection SCI. For mice in the SCI+Ctrl group (n>6), the BMS scores gradually improved to ˜3 (dorsal stepping) by 56 DPI (FIG. 11B). In contrast, mice in the SCI+Gsx1 group (n>6) had a significantly improved locomotor function with BMS score gradually increased from ˜0 to ˜5 by 30 DPI (FIG. 11B). Starting from ˜30 DPI, Gsx1-treated animals showed near normal locomotion (with BMS score reaching ˜6-7) compared to the Sham mice (FIG. 11B). These results demonstrate that Gsx1 treatment dramatically improved locomotor functional recovery after SCI (FIGS. 11A-11B).

To identify the molecular basis for the improved locomotor function, RT-qPCR analysis was used to assess expression of a selected set of genes involved in axon growth. Gsx1 treatment (n=4) significantly increased mRNA level of Ctnna1 and Col6a2 compared to the SCI+Ctrl group (n=4) at 35 DPI (FIG. 11C). RNA-SEQ, IPA, and gene ontology analysis on DEGs was performed. Gsx1 treatment led to the activation of Netrin signaling (FIG. 11D; FIG. 12B), and axonal guidance pathways (FIG. 11E), and CREB signaling in neurons (FIG. 12A). CREB is a transcription factor responsible for axon growth and regeneration 55. The RNA-Seq and IPA analysis further identified an increase in the expression of genes that promote synaptogenesis (FIG. 11F) and a decrease in the expression of genes known to inhibit synaptogenesis (FIG. 11G) in Gsx1 treatment at 35 DPI.

Neurotransmission of serotonin (5-HT) in the spinal cord is required for modulating sensory, motor, and autonomic functions. After SCI, 5-HT axons caudal to the injury site degenerate, while rostral to the injury site sprout. Therefore, the expression level of serotonergic neurons was measured using anti-5-HT antibody in spinal cord samples at 56 DPI to determine the effect of Gsx1 treatment on the recovery of 5-HT neuronal activity. Immunostaining results show that the level of 5-HT+ axons was similar in the region rostral to the injury site in both the control and Gsx1 treatment. While in the region caudal region, Gsx1 treatment increased the level of 5-HT axons (FIG. 12C). This result indicates that Gsx1 promotes 5-HT neuronal activity in the injured spinal cord. Lastly, gene ontology analysis revealed that DEGs were involved in cell communication, nervous system development, neurogenesis, and locomotion as key biological processes (FIG. 13). These results indicate that Gsx1 treatment upregulates signaling pathways associated with axon growth and guidance, which correlate with the improved locomotor function after SCI.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of treating a neurological disorder in a mammalian subject, comprising:
administering to the subject a therapeutically effective amount of Gsx1 protein or nucleic acid molecule encoding Gsx1, thereby treating the neurological disorder.

2. The method of claim 1, wherein the Gsx1 protein comprises a Gsx1-cell penetrating peptide (CPP) fusion protein.

3. The method of claim 1, wherein the Gsx1 protein comprises at least 80%, at least 85%, at least 90%, at least 95%, at least 98%, at least 99%, or 100% sequence identity to SEQ ID NO: 2 or 4, or wherein the nucleic acid molecule encoding Gsx1 encodes a protein comprising at least 80%, at least 85%, at least 90%, at least 95%, at least 98%, at least 99%, or 100% sequence identity to SEQ ID NO: 2 or 4.

4. The method of any claim 1, wherein the nucleic acid molecule encoding Gsx1 comprises at least 80%, at least 85%, at least 90%, at least 95%, at least 98%, at least 99%, or 100% sequence identity to SEQ ID NO: 1 or 3.

5. The method of claim 1, wherein the nucleic acid molecule encoding Gsx1 comprises a plasmid or viral vector.

6. The method of claim 5, wherein the viral vector is a lentiviral vector or adeno-associated viral vector.

7. The method of claim 1, wherein
the nucleic acid molecule encoding Gsx1 is operably linked to a promoter, and/or
wherein the promoter is operably linked to a neural-specific enhancer.

8. The method of claim 7, wherein the promoter is a constitutive promoter or a central nervous system (CNS)-specific promoter.

9. The method of claim 8, wherein the constitutive promoter is a CMV promoter.

10. The method of claim 8, wherein the CNS-specific promoter is a synapasin 1 (Syn1) promoter, glial fibrillary acidic protein (GFAP) promoter, nestin (NES) promoter, myelin-associated oligodendrocyte basic protein (MOBP) promoter, myelin basic protein (MBP) promoter, tyrosine hydroxylase (TH) promoter, or a forkhead box A2 (FOXA2) promoter.

11. The method of claim 2, wherein the cell penetrating peptide comprises at least 80%, at least 85%, at least 90%, at least 95%, at least 98%, at least 99%, or 100% sequence identity to any one of SEQ ID NOS: 61-79.

12. The method of claim 1, wherein the neurological disorder is a spinal cord injury, a brain injury, or both.

13. (canceled)

14. The method of claim 1, wherein the neurological disorder is Parkinson's disease, Alzheimer's disease, stroke, ischemia, epilepsy, Huntington's disease, multiple sclerosis, or amyotrophic lateral sclerosis.

15. The method of claim 1, wherein the administering comprises injection.

16. The method of claim 15, wherein the injection comprises injection into the CNS.

17. (canceled)

18. The method of claim 1, wherein the therapeutically effective amount of Gsx1 protein, or nucleic acid molecule encoding Gsx1, is present in a pharmaceutical composition.

19. The method of claim 1, wherein the administering comprises at least two separate administrations of the therapeutically effective amount of Gsx1 protein, or nucleic acid molecule encoding Gsx1.

20.23. (canceled)

24. A composition, comprising:
(1) an isolated Gsx1 protein comprising at least 80%, at least 85%, at least 90%, at least 95%, at least 98%, at least 99%, or 100% sequence identity to SEQ ID NO: 2 or 4;
an isolated Gsx1-cell penetrating peptide (CPP) fusion protein comprising a Gxx1 portion comprising at least 80%, at least 85%, at least 90%, at least 95%, at least 98%, at least 99%, or 100% sequence identity to SEQ ID NO: 2 or 4, and a CPP portion which optionally comprises at least 80%, at least 85%, at least 90%, at least 95%, at least 98%, at least 99%, or 100% sequence identity to any one of SEQ ID NOS: 61-79, wherein the Gsx1 portion and the CPP portion are optionally joined by a linker;
a nucleic acid molecule encoding a Gsx1 protein comprising at least 80%, at least 85%, at least 90%, at least 95%, at least 98%, at least 99%, or 100% sequence identity to SEQ ID NO: 1 or 3 or encoding a Gsx1 protein comprising at least 80%, at least 85%, at least 90%, at least 95%, at least 98%, at least 99%, or 100% sequence identity to SEQ ID NO: 2 or 4; or a nucleic acid molecule encoding a Gsx1-CPP fusion protein, wherein a Gsx1 portion of the Gsx1-CPP fusion protein is encoded by a nucleic acid molecule comprising at least 80%, at least 85%, at least 90%, at least 95%, at least 98%, at least 99%, or 100% sequence identity to SEQ ID NO: 1 or 3 or encodes a Gsx1 protein comprising at least 80%, at least 85%, at least 90%, at least 95%, at least 98%, at least 99%, or 100% sequence identity to SEQ ID NO: 2 or 4, and wherein optionally a nucleic acid molecule encoding a CPP portion of the Gsx1-CPP fusion protein encodes a protein comprising at least 80%, at least 85%, at least 90%, at least 95%, at least 98%, at least 99%, or 100% sequence identity to any one of SEQ ID NOS: 61-79; and
(2) a liposome,
wherein the Gsx1 protein, Gsx1-CPP fusion protein, nucleic acid molecule encoding Gsx1 protein, or nucleic acid molecule encoding Gsx1-CPP fusion protein, is encapsulated in the liposome.

25. A method of reprogramming a cell into a neuronal cell, comprising
introducing a nucleic acid molecule encoding Gsx1 or a Gsx1-CPP fusion protein, into a host cell, thereby reprogramming the host cell into a neuronal cell.

26. The method of claim 25, wherein the neuronal cell is a glutamatergic neuron or cholinergic neuron.

27. The method of claim 25, wherein the resulting reprogrammed cell is introduced into a subject with a neurological disorder.

28. The method of claim 25, wherein the host cell is a neural stem/progenitor cell (NSPC), fibroblast cell, or embryonic stem cell.


SEQUENCE LISTING (81 sequences)

SEQ ID NO: 1 - DNA, 847 bp, Homo sapiens
gtgggcgcagagggcgggctggctgcggggcgaccgcgcgccggggccatgccgcgctccttcctggtggactcgctagtgctgcgcgaggcgggcgagaagaaggcgcccgagggcagcccgccgccgctcttcccctacgctgtgcccccgccgcacgcgctgcacggtctctcgcctggcgcctgccacgcgcgcaaggctgggctgctgtgcgtgtgcccgctctgcgtcaccgcctcgcagctgcatgggccccccgggccgcccgcgctgcctctactcaaggcttccttcccacccttcggctcgcagtactgccacgcgcccctgggccgccagcactctgctgtgtcgcccggggtcgctcacggcccggccgccgctgctgctgccgccgcgctctaccagacctcctacccgctgcctgaccccaggcagttccactgcatctctgtggacagcagctctaaccagctgcccagcagcaagaggatgcgcacggctttcaccagcacgcagctgctagagctggagcgcgagttcgcttctaatatgtacctgtcccgcctacgtcgcatcgagatcgcgacctacctgaatctgtccgagaagcaggtgaagatctggtttcagaaccgccgagtgaagcacaagaaggagggcaagggcagcaaccatcgtggcggcggcggcgggggtgccggtggtggcgggagcgcaccgcaaggctgcaagtgcgcatcgctctcctcagccaagtgctccgaggatgacgacgaattgcccatgtctccgtcctcctcagggaaggacgaccgggatcttacggtcactccctaggcgc

SEQ ID NO: 2 - protein, 264 aa, Homo sapiens
MPRSFLVDSLVLREAGEKKAPEGSPPPLFPYAVPPPHALHGLSPGACHARKAGLLCVCPLCVTASQLHGPPGPPALPLLKASFPPFGSQYCHAPLGRQHSAVSPGVAHGPAAAAAAAALYQTSYPLPDPRQFHCISVDSSSNQLPSSKRMRTAFTSTQLLELEREFASNMYLSRLRRIEIATYLNLSEKQVKIWFQNRRVKHKKEGKGSNHRGGGGGGAGGGGSAPQGCKCASLSSAKCSEDDDELPMSPSSSGKDDRDLTVTP

SEQ ID NO: 3 - DNA, 1773 bp, Mus musculus
ccagcacctcgcccgctccgggaggtgcccgcagcagcagccaaggtgattccagcccgggcttgagccgcgcgtggagcctccggggcccgggaagctgcgggtggccgcggccaggggaagctacgacaggatctgcagttccctcgggctccaggggcgggctggcggcaggtggaccgcgcgccggagccatgccgcgctccttcctggtggattcccttgtgctgcgggaagccagcgacaagaaggctccggagggcagcccgccaccgctcttcccctacgcggtcccgccgccgcacgcgctccacggcctctcgccgggcgcctgccacgcgcgcaaggccggcttgctgtgcgtgtgtcccctctgtgtcaccgcttcgcagctgcacgggccccccgggccgccggcactgccgctactcaaggcgtccttccctcccttcggatcgcagtactgccacgcacccctgggccgccagcactccgtgtcccctggagtcgcccacggcccggctgcggccgcagcagctgctgcactctaccagacctcctacccgctgccggatcccagacagtttcactgcatctctgtggacagcagctcgaaccagctgcccagcagcaagaggatgcggacggcgttcaccagcacacagctcctggagctggagcgagagttcgcctccaacatgtacctctcccgcctgcggcgcatcgagatcgcgacctatctgaacctgtccgagaagcaggtgaagatctggtttcagaaccgccgggtgaagcacaagaaagaaggcaaaggcagtaaccaccgcggcggagctggggcgggggccggcgggggcgcaccgcaaggctgcaagtgctcttcgctctcctcagccaaatgctcagaggacgacgacgaattgcccatgtctccatcttcctccgggaaggatgacagagatctcacagtcactccgtaggtgcgccttttagaggaccattggtttccccaccccccaccccgacccttcccgcactggtccccaggcacccgctggccaaccgacggatttcgttgggctttgcggtggtgcgcagctttaggcagagctaagaccttagcagacacttgaagacagtgcccctgtcccttgggcttcagggtgtttaggaggactccaagcgatgaaggctgagtcctcctcctaggacacagcctcttctcccaggcacgcaggccggagcacagcgccttgctgaccgccagcgcctcttcgcctgccaactctgggctggttcaagcttcctcggttccactattctctccttctcggtcaatctgggcttttcactccgcgagtggctgtttgcttttcttttaacatttctttcttgcccccaactcgtctccccccactgtggtcctttatgcaacacgtctatggacttaacttttccttccctcctcaggaagtctctcccttctgtccgtttgtcccttaaacagggatcaggtcttgctgttcgaagaagtccccctagcagagagaactgtctcacatggtattgtattggggaaaatgactctgtttccaatacgctctaaacccccttcaaatgaagccgctgtaaccaacctctccccattgtccaggccccgactccctctggggactgactgactgtgtcattgtatcgtctctgtaatgccagaagatatttatttatttatttatgtacaaaattttaaataaactttttttttcttagaaa

SEQ ID NO: 4 - protein, 261 aa, Mus musculus
MPRSFLVDSLVLREASDKKAPEGSPPPLFPYAVPPPHALHGLSPGACHARKAGLLCVCPLCVTASQLHGPPGPPALPLLKASFPPFGSQYCHAPLGRQHSVSPGVAHGPAAAAAAAALYQTSYPLPDPRQFHCISVDSSSNQLPSSKRMRTAFTSTQLLELEREFASNMYLSRLRRIEIATYLNLSEKQVKIWFQNRRVKHKKEGKGSNHRGGAGAGAGGGAPQGCKCSSLSSAKCSEDDDELPMSPSSSGKDDRDLTVTP

SEQ ID NO: 5 - DNA, 19 bp, Artificial Sequence
  Synthetic oligonucleotide
cttccctcccttcggatcg

SEQ ID NO: 6 - DNA, 21 bp, Artificial Sequence
  Synthetic oligonucleotide
ggacccacaactgtcactcat

SEQ ID NO: 7 - DNA, 21 bp, Artificial Sequence
  Synthetic oligonucleotide
atggacgctgatggcaatacc

SEQ ID NO: 8 - DNA, 20 bp, Artificial Sequence
  Synthetic oligonucleotide
tgtttccgtggagacgcaag

SEQ ID NO: 9 - DNA, 22 bp, Artificial Sequence
  Synthetic oligonucleotide
gcaactgttcctgaactcaact

SEQ ID NO: 10 - DNA, 19 bp, Artificial Sequence
  Synthetic oligonucleotide
cctgtagcccacgtcgtag

SEQ ID NO: 11 - DNA, 22 bp, Artificial Sequence
  Synthetic oligonucleotide
atcattgaccgctcctttaggt

SEQ ID NO: 12 - DNA, 19 bp, Artificial Sequence
  Synthetic oligonucleotide
ccctgaagtcgaggagctg

SEQ ID NO: 13 - DNA, 20 bp, Artificial Sequence
  Synthetic oligonucleotide
aaccacgaactccacccttc

SEQ ID NO: 14 - DNA, 21 bp, Artificial Sequence
  Synthetic oligonucleotide
tggaaaatccctcggacagat

SEQ ID NO: 15 - DNA, 22 bp, Artificial Sequence
  Synthetic oligonucleotide
gtctcagagcaggataccaagc

SEQ ID NO: 16 - DNA, 23 bp, Artificial Sequence
  Synthetic oligonucleotide
aacaaagaccattcctccgaaag

SEQ ID NO: 17 - DNA, 20 bp, Artificial Sequence
  Synthetic oligonucleotide
ccattgtgaagcggtttggg

SEQ ID NO: 18 - DNA, 19 bp, Artificial Sequence
  Synthetic oligonucleotide
cggagacgcatcacctctg

SEQ ID NO: 19 - DNA, 19 bp, Artificial Sequence
  Synthetic oligonucleotide
gcaggtggtacgttgtggg

SEQ ID NO: 20 - DNA, 21 bp, Artificial Sequence
  Synthetic oligonucleotide
atttgtcccaatgtctgcgaa

SEQ ID NO: 21 - DNA, 19 bp, Artificial Sequence
  Synthetic oligonucleotide
cccttgctctgcctaacgc

SEQ ID NO: 22 - DNA, 21 bp, Artificial Sequence
  Synthetic oligonucleotide
aagctgttggtcaagttcgga

SEQ ID NO: 23 - DNA, 20 bp, Artificial Sequence
  Synthetic oligonucleotide
cctcgggtcagtttgagctg

SEQ ID NO: 24 - DNA, 21 bp, Artificial Sequence
  Synthetic oligonucleotide
cactgtccgtcaggatggaac

SEQ ID NO: 25 - DNA, 19 bp, Artificial Sequence
  Synthetic oligonucleotide
cccatccgattccccttcg

SEQ ID NO: 26 - DNA, 20 bp, Artificial Sequence
  Synthetic oligonucleotide
tcagcgagtgcatgaacgag

SEQ ID NO: 27 - DNA, 21 bp, Artificial Sequence
  Synthetic oligonucleotide
caggtctcctcatggctttgc

SEQ ID NO: 28 - DNA, 20 bp, Artificial Sequence
  Synthetic oligonucleotide
tgcaaggattcaccgaaagc

SEQ ID NO: 29 - DNA, 20 bp, Artificial Sequence
  Synthetic oligonucleotide
gctcctgattgggggactct

SEQ ID NO: 30 - DNA, 23 bp, Artificial Sequence
  Synthetic oligonucleotide
aagtctggagattaggactctgg

SEQ ID NO: 31 - DNA, 22 bp, Artificial Sequence
  Synthetic oligonucleotide
tgctaaacacagtcatttgcgt

SEQ ID NO: 32 - DNA, 22 bp, Artificial Sequence
  Synthetic oligonucleotide
agctcaacaaatcccagtctct

SEQ ID NO: 33 - DNA, 21 bp, Artificial Sequence
  Synthetic oligonucleotide
gtccacagagatgcagtgaaa

SEQ ID NO: 34 - DNA, 21 bp, Artificial Sequence
  Synthetic oligonucleotide
aagccccactttagctttacc

SEQ ID NO: 35 - DNA, 19 bp, Artificial Sequence
  Synthetic oligonucleotide
tccccattcacgtctccca

SEQ ID NO: 36 - DNA, 21 bp, Artificial Sequence
  Synthetic oligonucleotide
ttgagcctttgtaaatgggca

SEQ ID NO: 37 - DNA, 21 bp, Artificial Sequence
  Synthetic oligonucleotide
atcttttggggtccgtcaact

SEQ ID NO: 38 - DNA, 22 bp, Artificial Sequence
  Synthetic oligonucleotide
gggagtagacaaggtacaaccc

SEQ ID NO: 39 - DNA, 19 bp, Artificial Sequence
  Synthetic oligonucleotide
gctcgccttgatggttcct

SEQ ID NO: 40 - DNA, 19 bp, Artificial Sequence
  Synthetic oligonucleotide
ctgctgcacctctaagcga

SEQ ID NO: 41 - DNA, 21 bp, Artificial Sequence
  Synthetic oligonucleotide
gacctcaattttccgtccctc

SEQ ID NO: 42 - DNA, 21 bp, Artificial Sequence
  Synthetic oligonucleotide
catagcggagccttcttctca

SEQ ID NO: 43 - DNA, 21 bp, Artificial Sequence
  Synthetic oligonucleotide
ctctcctcgaataccacagcc

SEQ ID NO: 44 - DNA, 23 bp, Artificial Sequence
  Synthetic oligonucleotide
tgtaacaggctcacatgattctc

SEQ ID NO: 45 - DNA, 22 bp, Artificial Sequence
  Synthetic oligonucleotide
gccaggcggttgtttagataca

SEQ ID NO: 46 - DNA, 21 bp, Artificial Sequence
  Synthetic oligonucleotide
agggagtggaggagtcattcg

SEQ ID NO: 47 - DNA, 23 bp, Artificial Sequence
  Synthetic oligonucleotide
ctcttgtagctcatagatggtgc

SEQ ID NO: 48 - DNA, 23 bp, Artificial Sequence
  Synthetic oligonucleotide
tggctatcttggctataaagggg

SEQ ID NO: 49 - DNA, 19 bp, Artificial Sequence
  Synthetic oligonucleotide
ggagtcctggcatcgttgg

SEQ ID NO: 50 - DNA, 19 bp, Artificial Sequence
  Synthetic oligonucleotide
cgcacaccgaggtagttgg

SEQ ID NO: 51 - DNA, 22 bp, Artificial Sequence
  Synthetic oligonucleotide
ccttgaggcacactttgaagta

SEQ ID NO: 52 - DNA, 21 bp, Artificial Sequence
  Synthetic oligonucleotide
tagccgccaatcaggtttttg

SEQ ID NO: 53 - DNA, 22 bp, Artificial Sequence
  Synthetic oligonucleotide
ggttttctgttgcgaggtcatc

SEQ ID NO: 54 - DNA, 20 bp, Artificial Sequence
  Synthetic oligonucleotide
catggcgttgatctgggtca

SEQ ID NO: 55 - DNA, 21 bp, Artificial Sequence
  Synthetic oligonucleotide
cttccgaaaagaaggctgtcc

SEQ ID NO: 56 - DNA, 22 bp, Artificial Sequence
  Synthetic oligonucleotide
tgccatcaaagaacggacctat

SEQ ID NO: 57 - DNA, 22 bp, Artificial Sequence
  Synthetic oligonucleotide
ccaacacgaaatacacgttgac

SEQ ID NO: 58 - DNA, 23 bp, Artificial Sequence
  Synthetic oligonucleotide
acggcctctctttttattagacg

SEQ ID NO: 59 - DNA, 21 bp, Artificial Sequence
  Synthetic oligonucleotide
gcacacattctcatcgtccag

SEQ ID NO: 60 - DNA, 20 bp, Artificial Sequence
  Synthetic oligonucleotide
cggatggtctcagctttcac

SEQ ID NO: 61 - protein, 11 aa, Artificial Sequence
  Synthetic peptide
YGRKKRRQRRR

SEQ ID NO: 62 - protein, 18 aa, Artificial Sequence
  Synthetic peptide
RGGRLSYSRRRFSTSTGR

SEQ ID NO: 63 - protein, 10 aa, Artificial Sequence
  Synthetic peptide
RRLSYSRRRF

SEQ ID NO: 64 - protein, 12 aa, Artificial Sequence
  Synthetic peptide
PIRRRKKLRRLK

SEQ ID NO: 65 - protein, 12 aa, Artificial Sequence
  Synthetic peptide
RRQRRTSKLMKR

SEQ ID NO: 66 - protein, 15 aa, Artificial Sequence
  Synthetic peptide
RRRRNRTRRNRRRVR

SEQ ID NO: 67 - protein, 19 aa, Artificial Sequence
  Synthetic peptide
KMTRAQRRAAARRNRWTAR

SEQ ID NO: 68 - protein, 13 aa, Artificial Sequence
  Synthetic peptide
TRRQRTRRARRNR

SEQ ID NO: 69 - protein, 13 aa, Artificial Sequence
  Synthetic peptide
GRKKRRQRRRPPQ

SEQ ID NO: 70 - protein, 13 aa, Artificial Sequence
  Synthetic peptide
GRRRRRRRRRPPQ

SEQ ID NO: 71 - protein, 15 aa, Artificial Sequence
  Synthetic peptide
RQIKWFQNRRMKWKK

SEQ ID NO: 72 - protein, 17 aa, Artificial Sequence
  Synthetic peptide
KLALKLALKLALALKLA

SEQ ID NO: 73 - protein, 27 aa, Artificial Sequence
  Synthetic peptide
MGLGLHLLVLAAALQGAWSQPKKKRKV

SEQ ID NO: 74 - protein, 27 aa, Artificial Sequence
  Synthetic peptide
GALFLGWLGAAGSTMGAWSQPKKKRKV

SEQ ID NO: 75 - protein, 27 aa, Artificial Sequence
  Synthetic peptide
GALFLGFLGAAGSTMGAWSQPKKKRKV

SEQ ID NO: 76 - protein, 27 aa, Artificial Sequence
  Synthetic peptide
GALFLGFLGAAGSTMGAWSQPKSKRKV

SEQ ID NO: 77 - protein, 21 aa, Artificial Sequence
  Synthetic peptide
KETWFETWFTEWSQPKKKRKV

SEQ ID NO: 78 - protein, 27 aa, Artificial Sequence
  Synthetic peptide
GWTLNSAGYLLGKINLKALAALAKKIL

SEQ ID NO: 79 - protein, 21 aa, Artificial Sequence
  Synthetic peptide
KETWWETWWTEWSQPKKKRKV

SEQ ID NO: 80 - protein, 6 aa, Artificial Sequence
  Synthetic peptide
GSGSGS

SEQ ID NO: 81 - protein, 10 aa, Artificial Sequence
  Synthetic peptide
GGSGGGGSGG